SEQUENCE LISTING (77 sequences)

SEQ ID NO: 1 - DNA, 1973 bp, Human
ctgccggctcactcggctgctgcgtctggtctggcgtctgctgagaagatcctcttctaccctgctctgcacctgtgctcgactgccagccggctgagggcgggggtctccacggtggtcccagctcccaaggaggttgcagaagtaccgtacagagtggatttgcagggcagtggcatggagcccctcttccccgcgccgttctgggaggttatctacggcagccaccttcagggcaacctgtccctcctgagccccaaccacagtctgctgcccccgcatctgctgctcaatgccagccacggcgccttcctgcccctcgggctcaaggtcaccatcgtggggctctacctggccgtgtgtgtcggagggctcctggggaactgccttgtcatgtacgtcatcctcaggcacaccaaaatgaagacagccaccaatatttacatctttaacctggccctggccgacactctggtcctgctgacgctgcccttccagggcacggacatcctcctgggcttctggccgtttgggaatgcgctgtgcaagacagtcattgccattgactactacaacatgttcaccagcaccttcaccctaactgccatgagtgtggatcgctatgtagccatctgccaccccatccgtgccctcgacgtccgcacgtccagcaaagcccaggctgtcaatgtggccatctgggccctggcctctgttgtcggtgttcccgttgccatcatgggctcggcacaggtcgaggatgaagagatcgagtgcctggtggagatccctacccctcaggattactggggcccggtgtttgccatctgcatcttcctcttctccttcatcgtccccgtgctcgtcatctctgtctgctacagcctcatgatccggcggctccgtggagtccgcctgctctcgggctcccgagagaaggaccggaacctgcggcgcatcactcggctggtgctggtggtagtggctgtgttcgtgggctgctggacgcctgtccaggtcttcgtgctggcccaagggctgggggttcagccgagcagcgagactgccgtggccattctgcgcttctgcacggccctgggctacgtcaacagctgcctcaaccccatcctctacgccttcctggatgagaacttcaaggcctgcttccgcaagttctgctgtgcatctgccctgcgccgggacgtgcaggtgtctgaccgcgtgcgcagcattgccaaggacgtggccctggcctgcaagacctctgagacggtaccgcggcccgcatgactaggcgtggacctgcccatggtgcctgtcagcccgcagagcccatctacgcccaacacagagctcacacaggtcactgctctctaggcggacacaccctgggccctgagcatccagagcctgggatgggcttttccctgtgggccagggatgctcggtcccagaggaggacctagtgacatcatgggacaggtcaaagcattagggccacctccatggccccagacagactaaagctgccctcctggtgcagggccgaggggacacaaggacctacctggaagcagctgacatgctggtggacggccgttactggagcccgtgcccctccctccccgtgcttcatgtgactcttggcctctctgctgctgcgttggcagaaccctgggtgggcaggcacccggaggaggagcagcagctgtgtcatcctgtgccccccatgtgctgtgtgctgtttgcatggcagggctccagctgccttcagccctgtgacgtctcctcagggcagctggacaggcttggcacggcccgggaagtgcagcaggcagcttttctttggggtgggacttgccctgagcttggagctgccacctggaggacttgcctgttccgactccacctgtgcagccggggccaccccaggagaaagtgtccaggtgggggctggcagtccctggctgcag

SEQ ID NO: 2 - DNA, 23 bp, Artificial Sequence
  Primer M-572F
cacaggtcgaggatgaagagatc

SEQ ID NO: 3 - DNA, 23 bp, Artificial Sequence
  Primer M-714R
catgaggctgtagcagacagaga

SEQ ID NO: 4 - DNA, 26 bp, Artificial Sequence
  misc_binding: Probe M-658T, labeled 5′-terminal with FAM and 3′-terminal with TAMRA
ttcctcttctccttcatcgtccccgt

SEQ ID NO: 5 - DNA, 24 bp, Artificial Sequence
  Forward Primer
ccctgagagaagaagtgttaagga

SEQ ID NO: 6 - DNA, 18 bp, Artificial Sequence
  Reverse Primer
gcaaaaccccatgtcatg

SEQ ID NO: 7 - DNA, 28 bp, Artificial Sequence
  Probe
cgcagtgtggttagcttgacctttctgt

SEQ ID NO: 8 - DNA, 3663 bp, human
atgatgtttcgctcagatcgaatgtggagctgccattggaaatggaagcccagtcctctcctgttcttatttgctttatatatcatgtgtgttcctcactcagtgtggggatgtgccaactgccgagtggttttgtccaacccttctgggacctttacttctccatgctaccctaacgactacccaaacagccaggcttgcatgtggacgctccgagcccccaccggttatatcattcagataacatttaacgactttgacattgaagaagctcccaattgcatttatgactcattatcccttgataatggagagagccagactaaattttgtggagcaactgccaaaggcctatcatttaactcaagtgcgaatgagatgcatgtgtccttttcaagtgactttagcatccagaagaaaggtttcaatgccagctacatcagagttgccgtgtccttaaggaatcaaaaggtcattttaccccagacatcagatgcttaccaggtatctgttgcaaaaagcatctctattccagagctcagtgctttcacactctgctttgaagcaaccaaagttggccatgaagacagtgattggacagctttctcctactcaaatgcatccttcacacaattgctcagttttggaaaggccaagagtggctactttctatccatttctgattcaaaatgtttgttgaataatgcattacctgtcaaagaaaaagaagacatttttgcagaaagctttgaacagctctgccttgtttggaataattctttgggctctattggtgtaaatttcaaaagaaactatgaaacagttccatgtgattctaccattagtaaagttattcctgggaatgggaaattgttgttgggctccaatcaaaatgaaattgtctctctaaaaggggacatttataactttcgactttggaattttaccatgaatgccaaaatcctctccaacctcagctgtaatgtgaaagggaatgtagtcgactggcaaaatgacttctggaatatcccaaacctagctctgaaagctgaaagcaacctaagctgtggttcctacctgatcccgctcccagcagcagaactggccagctgtgcagacctggggaccctctgtcaagctactgtaaactctcctagtactacaccacccactgtcaccactaacatgcctgttactaacagaatcgataaacaaaggaatgatggaattatctatagaatatccgtagtgattcagaacatccttcgtcaccctgaggtaaaagtacagagcaaggtggcagaatggctcaattcaaccttccaaaattggaactacacggtttatgtcgttaatatcagttttcacctgagtgctggagaggacaagattaaagtcaagagaagccttgaggatgagccaaggttggtgctttgggcccttctagtttacaatgctaccaacaatactaatttagaaggaaaaatcattcagcagaagctcctaaaaaataatgagtccttggatgaaggcttgaggctacatacagtgaatgtgagacaactgggtcattgtcttgccatggaggaacccaaaggctactactggccatctatccaaccttctgaatacgttcttccttgtccagacaagcctggcttttctgcttctcggatatgtttttacaatgctaccaacccattggtaacctactggggacctgttgatatctccaactgtttaaaagaagcaaatgaagttgctaaccagattttaaatttaactgctgatgggcagaacttaacctcagccaatattaccaacattgtggaacaggtcaaaagaattgtgaataaagaagaaaacattgatataacacttggctcaactctaatgaatatattttctaatatcttaagcagttcagacagtgacttgcttgagtcatcttctgaagctttaaaaacaattgatgaattggccttcaagatagacctaaatagcacatcacatgtgaatattacaactcggaacttggctctcagcgtatcatccctgttaccagggacaaatgcaatttcaaattttagcattggtcttccaagcaataatgaatcgtatttccagatggattttgagagtggacaagtggatccactggcatctgtaattttgcctccaaacttacttgagaatttaagtccagaagattctgtattagttagaagagcacagtttactttcttcaacaaaactggacttttccaggatgtaggaccccaaagaaaaactttagtgagttatgtgatggcgtgcagtattggaaacattactatccagaatctgaaggatcctgttcaaataaaaatcaaacatacaagaactcaggaagtgcatcatcccatctgtgccttctgggatctgaacaaaaacaaaagttttggaggatggaacacgtcaggatgtgttgcacacagagattcagatgcaagtgagacagtctgcctgtgtaaccacttcacacactttggagttctgatggaccttccaagaagtgcctcacagttagatgcaagaaacactaaagtcctcactttcatcagctatattgggtgtggaatatctgctattttttcagcagcaactctcctgacatatgttgcttttgagaaattgcgaagggattatccctccaaaatcttgatgaacctgagcacagccctgctgttcctgaatctcctcttcctcctagatggctggatcacctccttcaatgtggatggactttgcattgctgttgcagtcctgttgcatttcttccttctggcaacctttacctggatggggctagaagcaattcacatgtacattgctctagttaaagtatttaacacttacattcgccgatacattctaaaattctgcatcattggctggggtttgcctgccttagtggtgtcagttgttctagcgagcagaaacaacaatgaagtctatggaaaagaaagttatgggaaagaaaaaggtgatgaattctgttggattcaagatccagtcatattttatgtgacctgtgctgggtattttggagtcatgttttttctgaacattgccatgttcattgtggtaatggtgcagatctgtgggaggaatggcaagagaagcaaccggaccctgagagaagaagtgttaaggaacctgcgcagtgtggttagcttgacctttctgttgggcatgacatggggttttgcattctttgcctggggacccttaaatatccccttcatgtacctcttctccatcttcaattcattacaaggcttatttatattcatcttccactgtgctatgaaggagaatgttcagaaacagtggcggcggcatctctgctgtggtagatttcggttagcagataactcagattggagtaagacagctaccaatatcatcaagaaaagttctgataatctaggaaaatctttgtcttcaagctccattggttccaactcaacctatcttacatccaaatctaaatccagctctaccacctatttcaaaaggaatagccacacagataatgtctcctatgagcattccttcaacaaaagtggatcactcagacagtgcttccatggacaagtccttgtcaaaactggcccatgc

SEQ ID NO: 9 - protein, 1221 aa, human
MMFRSDRMWSCHWKWKPSPLLFLFALYIMCVPHSVWGCANCRVVLSNPSGTFTSPCYPNDYPNSQACMWTLRAPTGYIIQITFNDFDIEEAPNCIYDSLSLDNGESQTKFCGATAKGLSFNSSANEMHVSFSSDFSIQKKGFNASYIRVAVSLRNQKVILPQTSDAYQVSVAKSISIPELSAFTLCFEATKVGHEDSDWTAFSYSNASFTQLLSFGKAKSGYFLSISDSKCLLNNALPVKEKEDIFAESFEQLCLVWNNSLGSIGVNFKRNYETVPCDSTISKVIPGNGKLLLGSNQNEIVSLKGDIYNFRLWNFTMNAKILSNLSCNVKGNVVDWQNDFWNIPNLALKAESNLSCGSYLIPLPAAELASCADLGTLCQATVNSPSTTPPTVTTNMPVTNRIDKQRNDGIIYRISVVIQNILRHPEVKVQSKVAEWLNSTFQNWNYTVYVVNISFHLSAGEDKIKVKRSLEDEPRLVLWALLVYNATNNTNLEGKIIQQKLLKNNESLDEGLRLHTVNVRQLGHCLAMEEPKGYYWPSIQPSEYVLPCPDKPGFSASRICFYNATNPLVTYWGPVDISNCLKEANEVANQILNLTADGQNLTSANITNIVEQVKRIVNKEENIDITLGSTLMNIFSNILSSSDSDLLESSSEALKTIDELAFKIDLNSTSHVNITTRNLALSVSSLLPGTNAISNFSIGLPSNNESYFQMDFESGQVDPLASVILPPNLLENLSPEDSVLVRRAQFTFFNKTGLFQDVGPQRKTLVSYVMACSIGNITIQNLKDPVQIKIKHTRTQEVHHPICAFWDLNKNKSFGGWNTSGCVAHRDSDASETVCLCNHFTHFGVLMDLPRSASQLDARNTKVLTFISYIGCGISAIFSAATLLTYVAFEKLRRDYPSKILMNLSTALLFLNLLFLLDGWITSFNVDGLCIAVAVLLHFFLLATFTWMGLEAIHMYIALVKVFNTYIRRYILKFCIIGWGLPALVVSVVLASRNNNEVYGKESYGKEKGDEFCWIQDPVIFYVTCAGYFGVMFFLNIAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHTDNVSYEHSFNKSGSLRQCFHGQVLVKTGPC

SEQ ID NO: 10 - DNA, 79 bp, human
ccctgagagaagaagtgttaaggaacctgcgcagtgtggttagcttgacctttctgttgggcatgacatggggttttgc

SEQ ID NO: 11 - DNA, 33 bp, Artificial Sequence
  Designed oligonucleotide primer to amplify DNA encoding dj287G14.2
ctcgagatgatgtttcgctcagatcgaatgtgg

SEQ ID NO: 12 - DNA, 33 bp, Artificial Sequence
  Designed oligonucleotide primer to amplify DNA encoding dj287G14.2
gctagctcagcatgggccagttttgacaaggac

SEQ ID NO: 13 - protein, 1193 aa, Mouse
MMFDTLGKRCCPWRLKPSALLFLFVLCVTCVPLSVCGCGSCRLVLSNPSGTFTSPCYPNDYPNTQSCSWTLRAPAGYIIQITFNDFDIEEAPNCIYDSLSLDNGESQTKFCGATAKGLSFNSSVNEMHVSFSSDFSIQKKGFNASYIRVAVSLRNQKVILPQTLDAYQVSVAKSISIPELKAFTLCFEASKVGNEGGDWTAFSYSDESLTQLLSLEKASNGYFLSISGSRCLLNNALPVKDKEDIFTENLEQLCLVWNNSWGSVGINFKKNYETVPCDSTISAVVPGDGTLLLGSDRDEIASLRGSIYNFRLWNFTMDLKALSNLSCSVSGNVIDWHNDFWSISTQALKAEGNLSCGSYLIQLPAAELTNCSELGTLCQATVSPPSTPPPTVTTNIPVTNRVHKQKNDGIMYRISVVIHNDFNHPEVKVQTKVAEWLNSTFQNWNYTVYVVNISFHQKVGEDRMKVKRDIMDDDKRLVLWALLVYNATNNVSLNEEKIKQKLMTNNASLEDGLRLCEVDVNQLGMCSALEDPDGFSWPATLPSVYKQPCPNKPGFFMTRACLSNGTSTFWGPVDTSNCSRQSNEVANEILNQTGDGQNLTSANINSIVEKVKRIVNKEENIDITLGSTLMNIFSNILSSSDSDLLESSTEALKTIDELAFKIDLNSTPHVNIETQNLALGVSSLIPGTNAPSNFSIGLPSNNESYFQMDFGNGQTDPLASVILPPNLLENLSPEDSVLVRRAQFTFFNKTGLFQDVGSQRKVLVSYVMACSIGNITIQNLKDPVQIKIKHTRTQEVHHPICAFWDMNKNKSFGGWNTSGCVAHSDLDAGETICLCSHFTHFGVLMDLPRSASQIDGRNTKVLTFITYIGCGISAIFSAATLLTYVAFEKLRRDYPSKILMNLSSALLFLNLIFLLDGWVTSFGVAGLCTAVAALLHFFLLATFTWMGLEAIHMYIALVKVFNTYIHRYILKFCIIGWGLPALVVSIILVSRRQNEVYGKESYGKDQDDEFCWIQDPVVFYVSCAGYFGVMFFLNVAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHSDNFS

SEQ ID NO: 14 - DNA, 3579 bp, Mouse
atgatgtttgacactctcgggaagaggtgctgcccttggagactgaagccaagcgccctgctgttcctgtttgttttatgtgttacctgtgttcctctctcagtgtgcggatgtggcagctgcagacttgtcctgtccaatccttccggtacctttacgtctccgtgttaccctaatgactaccctaatacccagtcttgttcgtggaccctccgagcccctgccggctacatcattcagataacgttcaatgacttcgacattgaagaagctcccaactgtatctatgactcattgtccctcgataatggagagagccagacaaaattctgtggagcgactgccaagggcctgtcatttaactccagcgtgaatgagatgcatgtgtccttttcaagtgactttagtatccagaagaaaggtttcaacgccagctacatcagagttgctgtgtccttgaggaatcaaaaggtcattttgccccagacattagatgcttaccaggtatcagttgcaaaaagcatctccattcctgaactcaaagctttcacgctctgttttgaagcctccaaagttggcaatgaaggtggtgactggacagctttctcctactcagacgagtcccttacacagctgctcagtcttgaaaaggccagtaatggctacttcctgtccatctctggctcaagatgcttgttgaacaatgcgttacctgtgaaggacaaagaggacatcttcacagaaaacttggagcagctctgtcttgtgtggaataattcttggggctccgttggtataaatttcaaaaagaactatgaaacagttccatgcgattccaccatcagtgctgtcgtacccggggatgggacattgctgttgggctccgacagagatgagatcgcctctctaaggggcagcatctataactttcgactttggaattttaccatggatctgaaagccctctccaacctcagctgtagtgtgtctgggaatgtcatagactggcacaatgacttttggagcatctcaacccaagctctgaaagccgagggcaacctgagctgtggttcctacctgatccagcttcctgcagcagagctgacaaactgttcagaactggggactctctgtcaagctactgtaagccctccttctactccaccacccactgtcaccactaatatccccgttactaacagagtccataaacaaaagaatgacggaataatgtatcgaatatctgttgtgattcacaatgactttaatcaccctgaagtaaaagtgcagaccaaagtagcagaatggctcaattcaacctttcagaattggaactacactgtttatgtggttaatataagttttcatcaaaaagtaggagaggacaggatgaaagtcaagagagacatcatggacgatgacaaaaggttggtgctctgggcccttctagtctacaatgctaccaataacgtcagcctgaatgaagagaagattaaacaaaagcttatgacaaataatgcatcactagaggatggactgaggctgtgtgaagtcgacgtgaaccagctgggtatgtgtagcgccttggaggatccagacggctttagttggccagccaccttaccctctgtctacaaacaaccatgtccaaacaagcctggcttttttatgactcgagcgtgcctttctaatggaacatcaaccttctggggccctgttgacacttccaactgttcaagacaatcaaatgaagtggccaatgagattttaaaccaaactggtgatgggcagaacctcacctcggctaatatcaacagcattgtagaaaaggtcaaacggatcgtgaacaaagaagaaaacattgacatcaccctcggctccactctaatgaatatattttctaatatcttaagcagttcagatagcgatttgcttgagtcttctactgaagctttaaaaacaattgacgagctagccttcaaaatagacctgaatagcaccccacatgtgaacattgagacacagaatttggcccttggagtctcatccctaattccaggaacaaatgcaccttcaaattttagcattggccttccaagcaataatgaatcgtacttccagatggacttcgggaacggacagacagatccactggcatctgtgattttgcctccaaatttgcttgagaatttaagccccgaagattctgtattggtcaggagagcacagttcactttcttcaacaaaaccggacttttccaggatgttggatctcaaagaaaagtcctcgtgagttatgtgatggcgtgcagcattggaaacattactatccagaatctgaaagatccggttcaaatcaaaatcaaacacaccagaacacaggaagtgcatcatcctatctgtgccttctgggatatgaacaaaaacaaaagtttcggggggtggaacacctcaggatgtgttgcccactctgatttggacgctggtgagaccatttgtctgtgcagccacttcactcactttggagttctgatggatcttccaaggagtgcctcacaaatagatggaagaaacacaaaagtcctcacgttcattacctatattgggtgcggaatatctgccattttctcagctgcaactctcctgacatatgttgcttttgagaagctgcgcagggattatccctccaaaatcctgatgaatctgagctcggccttgctcttcctgaatctcatcttcctcctggatggctgggtcacttcctttggcgtggctggactctgcacggctgtggctgccctgttgcacttcttcctcctggctaccttcacctggatggggctggaagccatccacatgtacattgctcttgtgaaagtgtttaacacttacatccaccgctatattctaaaattctgcatcataggctggggtctgccagccttggtggtgtcaattattctagtgagcagaagacaaaatgaagtatatggaaaagaaagttatgggaaagatcaggatgatgaattctgctggattcaggatcctgtggtgttttatgtgagctgtgccgggtacttcggagtcatgttcttcctgaatgtcgccatgttcattgtggtcatggtgcagatctgtgggaggaatggaaagagaagcaaccggaccctgagagaagaggttttaagaaacctgcgcagtgtggtcagcctgaccttcctgcttggcatgacgtgggggtttgctttctttgcctggggacccttaaatattcctttcatgtacctcttctccatcttcaattcattacaaggtttatttatattcatcttccactgtgcgatgaaggagaatgttcagaaacagtggaggcgtcacctctgctgtggcaggtttcggctagcagacaactcagattggagtaagacagctaccaatatcatcaagaagagctccgataacctggggaaatctttgtcttcaagctccattggctccaattcaacatatctcacatccaaatcaaagtccagctccactacctatttcaaaagaaacagccactcggataatttctcc

SEQ ID NO: 15 - protein, 1248 aa, Mouse
MMFDTLGKRCCPWRLKPSALLFLFVLCVTCVPLSVCGCGSCRLVLSNPSGTFTSPCYPNDYPNTQSCSWTLRAPAGYIIQITFNDFDIEEAPNCIYDSLSLDNGESQTKFCGATAKGLSFNSSVNEMHVSFSSDFSIQKKGFNASYIRVAVSLRNQKVILPQTLDAYQVSVAKSISIPELKAFTLCFEASKVGNEGGDWTAFSYSDESLTQLLSLEKASNGYFLSISGSRCLLNNALPVKDKEDIFTENLEQLCLVWNNSWGSVGINFKKNYETVPCDSTISAVVPGDGTLLLGSDRDEIASLRGSIYNFRLWNFTMDLKALSNLSCSVSGNVIDWHNDFWSISTQALKAEGNLSCGSYLIQLPAAELTNCSELGTLCQATVSPPSTPPPTVTTNIPVTNRVHKQKNDGIMYRISVVIHNDFNHPEVKVQTKVAEWLNSTFQNWNYTVYVVNISFHQKVGEDRMKVKRDIMDDDKRLVLWALLVYNATNNVSLNEEKIKQKLMTNNASLEDGLRLCEVDVNQLGMCSALEDPDGFSWPATLPSVYKQPCPNKPGFFMTRACLSNGTSTFWGPVDTSNCSRQSNEVANEILNQTGDGQNLTSANINSIVEKVKRIVNKEENIDITLGSTLMNIFSNILSSSDSDLLESSTEALKTIDELAFKIDLNSTPHVNIETQNLALGVSSLIPGTNAPSNFSIGLPSNNESYFQMDFGNGQTDPLASVILPPNLLENLSPEDSVLVRRAQFTFFNKTGLFQDVGSQRKVLVSYVMACSIGNITIQNLKDPVQIKIKHTRTQEVHHPICAFWDMNKNKSFGGWNTSGCVAHSDLDAGETICLCSHFTHFGVLMDLPRSASQIDGRNTKVLTFITYIGCGISAIFSAATLLTYVAFEKLRRDYPSKILMNLSSALLFLNLIFLLDGWVTSFGVAGLCTAVAALLHFFLLATFTWMGLEAIHMYIALVKVFNTYIHRYILKFCIIGWGLPALVVSIILVSRRQNEVYGKESYGKDQDDEFCWIQDPVVFYVSCAGYFGVMFFLNVAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHSDSTSTDKSLSKLTHAGGEQTSIIPVHQVIDKVKGYCNAHSDNFYKNIILSDAFSHSTKF

SEQ ID NO: 16 - DNA, 3744 bp, Mouse
atgatgtttgacactctcgggaagaggtgctgcccttggagactgaagccaagcgccctgctgttcctgtttgttttatgtgttacctgtgttcctctctcagtgtgcggatgtggcagctgcagacttgtcctgtccaatccttccggtacctttacgtctccgtgttaccctaatgactaccctaatacccagtcttgttcgtggaccctccgagcccctgccggctacatcattcagataacgttcaatgacttcgacattgaagaagctcccaactgtatctatgactcattgtccctcgataatggagagagccagacaaaattctgtggagcgactgccaagggcctgtcatttaactccagcgtgaatgagatgcatgtgtccttttcaagtgactttagtatccagaagaaaggtttcaacgccagctacatcagagttgctgtgtccttgaggaatcaaaaggtcattttgccccagacattagatgcttaccaggtatcagttgcaaaaagcatctccattcctgaactcaaagctttcacgctctgttttgaagcctccaaagttggcaatgaaggtggtgactggacagctttctcctactcagacgagtcccttacacagctgctcagtcttgaaaaggccagtaatggctacttcctgtccatctctggctcaagatgcttgttgaacaatgcgttacctgtgaaggacaaagaggacatcttcacagaaaacttggagcagctctgtcttgtgtggaataattcttggggctccgttggtataaatttcaaaaagaactatgaaacagttccatgcgattccaccatcagtgctgtcgtacccggggatgggacattgctgttgggctccgacagagatgagatcgcctctctaaggggcagcatctataactttcgactttggaattttaccatggatctgaaagccctctccaacctcagctgtagtgtgtctgggaatgtcatagactggcacaatgacttttggagcatctcaacccaagctctgaaagccgagggcaacctgagctgtggttcctacctgatccagcttcctgcagcagagctgacaaactgttcagaactggggactctctgtcaagctactgtaagccctccttctactccaccacccactgtcaccactaatatccccgttactaacagagtccataaacaaaagaatgacggaataatgtatcgaatatctgttgtgattcacaatgactttaatcaccctgaagtaaaagtgcagaccaaagtagcagaatggctcaattcaacctttcagaattggaactacactgtttatgtggttaatataagttttcatcaaaaagtaggagaggacaggatgaaagtcaagagagacatcatggacgatgacaaaaggttggtgctctgggcccttctagtctacaatgctaccaataacgtcagcctgaatgaagagaagattaaacaaaagcttatgacaaataatgcatcactagaggatggactgaggctgtgtgaagtcgacgtgaaccagctgggtatgtgtagcgccttggaggatccagacggctttagttggccagccaccttaccctctgtctacaaacaaccatgtccaaacaagcctggcttttttatgactcgagcgtgcctttctaatggaacatcaaccttctggggccctgttgacacttccaactgttcaagacaatcaaatgaagtggccaatgagattttaaaccaaactggtgatgggcagaacctcacctcggctaatatcaacagcattgtagaaaaggtcaaacggatcgtgaacaaagaagaaaacattgacatcaccctcggctccactctaatgaatatattttctaatatcttaagcagttcagatagcgatttgcttgagtcttctactgaagctttaaaaacaattgacgagctagccttcaaaatagacctgaatagcaccccacatgtgaacattgagacacagaatttggcccttggagtctcatccctaattccaggaacaaatgcaccttcaaattttagcattggccttccaagcaataatgaatcgtacttccagatggacttcgggaacggacagacagatccactggcatctgtgattttgcctccaaatttgcttgagaatttaagccccgaagattctgtattggtcaggagagcacagttcactttcttcaacaaaaccggacttttccaggatgttggatctcaaagaaaagtcctcgtgagttatgtgatggcgtgcagcattggaaacattactatccagaatctgaaagatccggttcaaatcaaaatcaaacacaccagaacacaggaagtgcatcatcctatctgtgccttctgggatatgaacaaaaacaaaagtttcggggggtggaacacctcaggatgtgttgcccactctgatttggacgctggtgagaccatttgtctgtgcagccacttcactcactttggagttctgatggatcttccaaggagtgcctcacaaatagatggaagaaacacaaaagtcctcacgttcattacctatattgggtgcggaatatctgccattttctcagctgcaactctcctgacatatgttgcttttgagaagctgcgcagggattatccctccaaaatcctgatgaatctgagctcggccttgctcttcctgaatctcatcttcctcctggatggctgggtcacttcctttggcgtggctggactctgcacggctgtggctgccctgttgcacttcttcctcctggctaccttcacctggatggggctggaagccatccacatgtacattgctcttgtgaaagtgtttaacacttacatccaccgctatattctaaaattctgcatcataggctggggtctgccagccttggtggtgtcaattattctagtgagcagaagacaaaatgaagtatatggaaaagaaagttatgggaaagatcaggatgatgaattctgctggattcaggatcctgtggtgttttatgtgagctgtgccgggtacttcggagtcatgttcttcctgaatgtcgccatgttcattgtggtcatggtgcagatctgtgggaggaatggaaagagaagcaaccggaccctgagagaagaggttttaagaaacctgcgcagtgtggtcagcctgaccttcctgcttggcatgacgtgggggtttgctttctttgcctggggacccttaaatattcctttcatgtacctcttctccatcttcaattcattacaaggtttatttatattcatcttccactgtgcgatgaaggagaatgttcagaaacagtggaggcgtcacctctgctgtggcaggtttcggctagcagacaactcagattggagtaagacagctaccaatatcatcaagaagagctccgataacctggggaaatctttgtcttcaagctccattggctccaattcaacatatctcacatccaaatcaaagtccagctccactacctatttcaaaagaaacagccactcggacagcacttccacggacaagtccctgtcaaaactgacccatgctggtggagaacagacgtcaatcattcccgtccatcaggttattgataaggtcaagggttactgtaatgcccattccgataacttctataaaaatatcatcctgtcagatgccttcagccacagcacaaagttt

SEQ ID NO: 17 - protein, 1203 aa, Mouse
MSVRPLGISTRMFDTLGKRCCPWRLKPSALLFLFVLCVTCVPLSVCGCGSCRLVLSNPSGTFTSPCYPNDYPNTQSCSWTLRAPAGYIIQITFNDFDIEEAPNCIYDSLSLDNGESQTKFCGATAKGLSFNSSVNEMHVSFSSDFSIQKKGFNASYIRVAVSLRNQKVILPQTLDAYQVSVAKSISIPELKAFTLCFEASKVGNEGGDWTAFSYSDESLTQLLSLEKASNGYFLSISGSRCLLNNALPVKDKEDIFTENLEQLCLVWNNSWGSVGINFKKNYETVPCDSTISAVVPGDGTLLLGSDRDEIASLRGSIYNFRLWNFTMDLKALSNLSCSVSGNVIDWHNDFWSISTQALKAEGNLSCGSYLIQLPAAELTNCSELGTLCQATVSPPSTPPPTVTTNIPVTNRVHKQKNDGIMYRISVVIHNDFNHPEVKVQTKVAEWLNSTFQNWNYTVYVVNISFHQKVGEDRMKVKRDIMDDDKRLVLWALLVYNATNNVSLNEEKIKQKLMTNNASLEDGLRLCEVDVNQLGMCSALEDPDGFSWPATLPSVYKQPCPNKPGFFMTRACLSNGTSTFWGPVDTSNCSRQSNEVANEILNQTGDGQNLTSANINSIVEKVKRIVNKEENIDITLGSTLMNIFSNILSSSDSDLLESSTEALKTIDELAFKIDLNSTPHVNIETQNLALGVSSLIPGTNAPSNFSIGLPSNNESYFQMDFGNGQTDPLASVILPPNLLENLSPEDSVLVRRAQFTFFNKTGLFQDVGSQRKVLVSYVMACSIGNITIQNLKDPVQIKIKHTRTQEVHHPICAFWDMNKNKSFGGWNTSGCVAHSDLDAGETICLCSHFTHFGVLMDLPRSASQIDGRNTKVLTFITYIGCGISAIFSAATLLTYVAFEKLRRDYPSKILMNLSSALLFLNLIFLLDGWVTSFGVAGLCTAVAALLHFFLLATFTWMGLEAIHMYIALVKVFNTYIHRYILKFCIIGWGLPALVVSIILVSRRQNEVYGKESYGKDQDDEFCWIQDPVVFYVSCAGYFGVMFFLNVAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHSDNFS

SEQ ID NO: 18 - DNA, 3609 bp, Mouse
atgagtgtgcggcctctgggaatctccacacggatgtttgacactctcgggaagaggtgctgcccttggagactgaagccaagcgccctgctgttcctgtttgttttatgtgttacctgtgttcctctctcagtgtgcggatgtggcagctgcagacttgtcctgtccaatccttccggtacctttacgtctccgtgttaccctaatgactaccctaatacccagtcttgttcgtggaccctccgagcccctgccggctacatcattcagataacgttcaatgacttcgacattgaagaagctcccaactgtatctatgactcattgtccctcgataatggagagagccagacaaaattctgtggagcgactgccaagggcctgtcatttaactccagcgtgaatgagatgcatgtgtccttttcaagtgactttagtatccagaagaaaggtttcaacgccagctacatcagagttgctgtgtccttgaggaatcaaaaggtcattttgccccagacattagatgcttaccaggtatcagttgcaaaaagcatctccattcctgaactcaaagctttcacgctctgttttgaagcctccaaagttggcaatgaaggtggtgactggacagctttctcctactcagacgagtcccttacacagctgctcagtcttgaaaaggccagtaatggctacttcctgtccatctctggctcaagatgcttgttgaacaatgcgttacctgtgaaggacaaagaggacatcttcacagaaaacttggagcagctctgtcttgtgtggaataattcttggggctccgttggtataaatttcaaaaagaactatgaaacagttccatgcgattccaccatcagtgctgtcgtacccggggatgggacattgctgttgggctccgacagagatgagatcgcctctctaaggggcagcatctataactttcgactttggaattttaccatggatctgaaagccctctccaacctcagctgtagtgtgtctgggaatgtcatagactggcacaatgacttttggagcatctcaacccaagctctgaaagccgagggcaacctgagctgtggttcctacctgatccagcttcctgcagcagagctgacaaactgttcagaactggggactctctgtcaagctactgtaagccctccttctactccaccacccactgtcaccactaatatccccgttactaacagagtccataaacaaaagaatgacggaataatgtatcgaatatctgttgtgattcacaatgactttaatcaccctgaagtaaaagtgcagaccaaagtagcagaatggctcaattcaacctttcagaattggaactacactgtttatgtggttaatataagttttcatcaaaaagtaggagaggacaggatgaaagtcaagagagacatcatggacgatgacaaaaggttggtgctctgggcccttctagtctacaatgctaccaataacgtcagcctgaatgaagagaagattaaacaaaagcttatgacaaataatgcatcactagaggatggactgaggctgtgtgaagtcgacgtgaaccagctgggtatgtgtagcgccttggaggatccagacggctttagttggccagccaccttaccctctgtctacaaacaaccatgtccaaacaagcctggcttttttatgactcgagcgtgcctttctaatggaacatcaaccttctggggccctgttgacacttccaactgttcaagacaatcaaatgaagtggccaatgagattttaaaccaaactggtgatgggcagaacctcacctcggctaatatcaacagcattgtagaaaaggtcaaacggatcgtgaacaaagaagaaaacattgacatcaccctcggctccactctaatgaatatattttctaatatcttaagcagttcagatagcgatttgcttgagtcttctactgaagctttaaaaacaattgacgagctagccttcaaaatagacctgaatagcaccccacatgtgaacattgagacacagaatttggcccttggagtctcatccctaattccaggaacaaatgcaccttcaaattttagcattggccttccaagcaataatgaatcgtacttccagatggacttcgggaacggacagacagatccactggcatctgtgattttgcctccaaatttgcttgagaatttaagccccgaagattctgtattggtcaggagagcacagttcactttcttcaacaaaaccggacttttccaggatgttggatctcaaagaaaagtcctcgtgagttatgtgatggcgtgcagcattggaaacattactatccagaatctgaaagatccggttcaaatcaaaatcaaacacaccagaacacaggaagtgcatcatcctatctgtgccttctgggatatgaacaaaaacaaaagtttcggggggtggaacacctcaggatgtgttgcccactctgatttggacgctggtgagaccatttgtctgtgcagccacttcactcactttggagttctgatggatcttccaaggagtgcctcacaaatagatggaagaaacacaaaagtcctcacgttcattacctatattgggtgcggaatatctgccattttctcagctgcaactctcctgacatatgttgcttttgagaagctgcgcagggattatccctccaaaatcctgatgaatctgagctcggccttgctcttcctgaatctcatcttcctcctggatggctgggtcacttcctttggcgtggctggactctgcacggctgtggctgccctgttgcacttcttcctcctggctaccttcacctggatggggctggaagccatccacatgtacattgctcttgtgaaagtgtttaacacttacatccaccgctatattctaaaattctgcatcataggctggggtctgccagccttggtggtgtcaattattctagtgagcagaagacaaaatgaagtatatggaaaagaaagttatgggaaagatcaggatgatgaattctgctggattcaggatcctgtggtgttttatgtgagctgtgccgggtacttcggagtcatgttcttcctgaatgtcgccatgttcattgtggtcatggtgcagatctgtgggaggaatggaaagagaagcaaccggaccctgagagaagaggttttaagaaacctgcgcagtgtggtcagcctgaccttcctgcttggcatgacgtgggggtttgctttctttgcctggggacccttaaatattcctttcatgtacctcttctccatcttcaattcattacaaggtttatttatattcatcttccactgtgcgatgaaggagaatgttcagaaacagtggaggcgtcacctctgctgtggcaggtttcggctagcagacaactcagattggagtaagacagctaccaatatcatcaagaagagctccgataacctggggaaatctttgtcttcaagctccattggctccaattcaacatatctcacatccaaatcaaagtccagctccactacctatttcaaaagaaacagccactcggataatttctcc

SEQ ID NO: 19 - protein, 1258 aa, Mouse
MSVRPLGISTRMFDTLGKRCCPWRLKPSALLFLFVLCVTCVPLSVCGCGSCRLVLSNPSGTFTSPCYPNDYPNTQSCSWTLRAPAGYIIQITFNDFDIEEAPNCIYDSLSLDNGESQTKFCGATAKGLSFNSSVNEMHVSFSSDFSIQKKGFNASYIRVAVSLRNQKVILPQTLDAYQVSVAKSISIPELKAFTLCFEASKVGNEGGDWTAFSYSDESLTQLLSLEKASNGYFLSISGSRCLLNNALPVKDKEDIFTENLEQLCLVWNNSWGSVGINFKKNYETVPCDSTISAVVPGDGTLLLGSDRDEIASLRGSIYNFRLWNFTMDLKALSNLSCSVSGNVIDWHNDFWSISTQALKAEGNLSCGSYLIQLPAAELTNCSELGTLCQATVSPPSTPPPTVTTNIPVTNRVHKQKNDGIMYRISVVIHNDFNHPEVKVQTKVAEWLNSTFQNWNYTVYVVNISFHQKVGEDRMKVKRDIMDDDKRLVLWALLVYNATNNVSLNEEKIKQKLMTNNASLEDGLRLCEVDVNQLGMCSALEDPDGFSWPATLPSVYKQPCPNKPGFFMTRACLSNGTSTFWGPVDTSNCSRQSNEVANEILNQTGDGQNLTSANINSIVEKVKRIVNKEENIDITLGSTLMNIFSNILSSSDSDLLESSTEALKTIDELAFKIDLNSTPHVNIETQNLALGVSSLIPGTNAPSNFSIGLPSNNESYFQMDFGNGQTDPLASVILPPNLLENLSPEDSVLVRRAQFTFFNKTGLFQDVGSQRKVLVSYVMACSIGNITIQNLKDPVQIKIKHTRTQEVHHPICAFWDMNKNKSFGGWNTSGCVAHSDLDAGETICLCSHFTHFGVLMDLPRSASQIDGRNTKVLTFITYIGCGISAIFSAATLLTYVAFEKLRRDYPSKILMNLSSALLFLNLIFLLDGWVTSFGVAGLCTAVAALLHFFLLATFTWMGLEAIHMYIALVKVFNTYIHRYILKFCIIGWGLPALVVSIILVSRRQNEVYGKESYGKDQDDEFCWIQDPVVFYVSCAGYFGVMFFLNVAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHSDSTSTDKSLSKLTHAGGEQTSIIPVHQVIDKVKGYCNAHSDNFYKNIILSDAFSHSTKF

SEQ ID NO: 20 - DNA, 3774 bp, Mouse
atgagtgtgcggcctctgggaatctccacacggatgtttgacactctcgggaagaggtgctgcccttggagactgaagccaagcgccctgctgttcctgtttgttttatgtgttacctgtgttcctctctcagtgtgcggatgtggcagctgcagacttgtcctgtccaatccttccggtacctttacgtctccgtgttaccctaatgactaccctaatacccagtcttgttcgtggaccctccgagcccctgccggctacatcattcagataacgttcaatgacttcgacattgaagaagctcccaactgtatctatgactcattgtccctcgataatggagagagccagacaaaattctgtggagcgactgccaagggcctgtcatttaactccagcgtgaatgagatgcatgtgtccttttcaagtgactttagtatccagaagaaaggtttcaacgccagctacatcagagttgctgtgtccttgaggaatcaaaaggtcattttgccccagacattagatgcttaccaggtatcagttgcaaaaagcatctccattcctgaactcaaagctttcacgctctgttttgaagcctccaaagttggcaatgaaggtggtgactggacagctttctcctactcagacgagtcccttacacagctgctcagtcttgaaaaggccagtaatggctacttcctgtccatctctggctcaagatgcttgttgaacaatgcgttacctgtgaaggacaaagaggacatcttcacagaaaacttggagcagctctgtcttgtgtggaataattcttggggctccgttggtataaatttcaaaaagaactatgaaacagttccatgcgattccaccatcagtgctgtcgtacccggggatgggacattgctgttgggctccgacagagatgagatcgcctctctaaggggcagcatctataactttcgactttggaattttaccatggatctgaaagccctctccaacctcagctgtagtgtgtctgggaatgtcatagactggcacaatgacttttggagcatctcaacccaagctctgaaagccgagggcaacctgagctgtggttcctacctgatccagcttcctgcagcagagctgacaaactgttcagaactggggactctctgtcaagctactgtaagccctccttctactccaccacccactgtcaccactaatatccccgttactaacagagtccataaacaaaagaatgacggaataatgtatcgaatatctgttgtgattcacaatgactttaatcaccctgaagtaaaagtgcagaccaaagtagcagaatggctcaattcaacctttcagaattggaactacactgtttatgtggttaatataagttttcatcaaaaagtaggagaggacaggatgaaagtcaagagagacatcatggacgatgacaaaaggttggtgctctgggcccttctagtctacaatgctaccaataacgtcagcctgaatgaagagaagattaaacaaaagcttatgacaaataatgcatcactagaggatggactgaggctgtgtgaagtcgacgtgaaccagctgggtatgtgtagcgccttggaggatccagacggctttagttggccagccaccttaccctctgtctacaaacaaccatgtccaaacaagcctggcttttttatgactcgagcgtgcctttctaatggaacatcaaccttctggggccctgttgacacttccaactgttcaagacaatcaaatgaagtggccaatgagattttaaaccaaactggtgatgggcagaacctcacctcggctaatatcaacagcattgtagaaaaggtcaaacggatcgtgaacaaagaagaaaacattgacatcaccctcggctccactctaatgaatatattttctaatatcttaagcagttcagatagcgatttgcttgagtcttctactgaagctttaaaaacaattgacgagctagccttcaaaatagacctgaatagcaccccacatgtgaacattgagacacagaatttggcccttggagtctcatccctaattccaggaacaaatgcaccttcaaattttagcattggccttccaagcaataatgaatcgtacttccagatggacttcgggaacggacagacagatccactggcatctgtgattttgcctccaaatttgcttgagaatttaagccccgaagattctgtattggtcaggagagcacagttcactttcttcaacaaaaccggacttttccaggatgttggatctcaaagaaaagtcctcgtgagttatgtgatggcgtgcagcattggaaacattactatccagaatctgaaagatccggttcaaatcaaaatcaaacacaccagaacacaggaagtgcatcatcctatctgtgccttctgggatatgaacaaaaacaaaagtttcggggggtggaacacctcaggatgtgttgcccactctgatttggacgctggtgagaccatttgtctgtgcagccacttcactcactttggagttctgatggatcttccaaggagtgcctcacaaatagatggaagaaacacaaaagtcctcacgttcattacctatattgggtgcggaatatctgccattttctcagctgcaactctcctgacatatgttgcttttgagaagctgcgcagggattatccctccaaaatcctgatgaatctgagctcggccttgctcttcctgaatctcatcttcctcctggatggctgggtcacttcctttggcgtggctggactctgcacggctgtggctgccctgttgcacttcttcctcctggctaccttcacctggatggggctggaagccatccacatgtacattgctcttgtgaaagtgtttaacacttacatccaccgctatattctaaaattctgcatcataggctggggtctgccagccttggtggtgtcaattattctagtgagcagaagacaaaatgaagtatatggaaaagaaagttatgggaaagatcaggatgatgaattctgctggattcaggatcctgtggtgttttatgtgagctgtgccgggtacttcggagtcatgttcttcctgaatgtcgccatgttcattgtggtcatggtgcagatctgtgggaggaatggaaagagaagcaaccggaccctgagagaagaggttttaagaaacctgcgcagtgtggtcagcctgaccttcctgcttggcatgacgtgggggtttgctttctttgcctggggacccttaaatattcctttcatgtacctcttctccatcttcaattcattacaaggtttatttatattcatcttccactgtgcgatgaaggagaatgttcagaaacagtggaggcgtcacctctgctgtggcaggtttcggctagcagacaactcagattggagtaagacagctaccaatatcatcaagaagagctccgataacctggggaaatctttgtcttcaagctccattggctccaattcaacatatctcacatccaaatcaaagtccagctccactacctatttcaaaagaaacagccactcggacagcacttccacggacaagtccctgtcaaaactgacccatgctggtggagaacagacgtcaatcattcccgtccatcaggttattgataaggtcaagggttactgtaatgcccattccgataacttctataaaaatatcatcctgtcagatgccttcagccacagcacaaagttt

SEQ ID NO: 21 - protein, 1165 aa, Mouse
MMFDTLGKRCCPWRLKPSALLFLFVLCVTCVPLSVCGCGSCRLVLSNPSGTFTSPCYPNDYPNTQSCSWTLRAPAGYIIQITFNDFDIEEAPNCIYDSLSLDNGESQTKFCGATAKGLSFNSSVNEMHVSFSSDFSIQKKGFNASYIRVAVSLRNQKVILPQTLDAYQVSVAKSISIPELKAFTLCFEASKVGNEGGDWTAFSYSDESLTQLLSLEKASNGYFLSISGSRCLLNNALPVKDKEDIFTENLEQLCLVWNNSWGSVGINFKKNYETVPCDSTISAVVPGDGTLLLGSDRDEIASLRGSIYNFRLWNFTMDLKALSNLSCSVSGNVIDWHNDFWSISTQALKAEGNLSCGSYLIQLPAAELTNCSELGTLCQDGIMYRISVVIHNDFNHPEVKVQTKVAEWLNSTFQNWNYTVYVVNISFHQKVGEDRMKVKRDIMDDDKRLVLWALLVYNATNNVSLNEEKIKQKLMTNNASLEDGLRLCEVDVNQLGMCSALEDPDGFSWPATLPSVYKQPCPNKPGFFMTRACLSNGTSTFWGPVDTSNCSRQSNEVANEILNQTGDGQNLTSANINSIVEKVKRIVNKEENIDITLGSTLMNIFSNILSSSDSDLLESSTEALKTIDELAFKIDLNSTPHVNIETQNLALGVSSLIPGTNAPSNFSIGLPSNNESYFQMDFGNGQTDPLASVILPPNLLENLSPEDSVLVRRAQFTFFNKTGLFQDVGSQRKVLVSYVMACSIGNITIQNLKDPVQIKIKHTRTQEVHHPICAFWDMNKNKSFGGWNTSGCVAHSDLDAGETICLCSHFTHFGVLMDLPRSASQIDGRNTKVLTFITYIGCGISAIFSAATLLTYVAFEKLRRDYPSKILMNLSSALLFLNLIFLLDGWVTSFGVAGLCTAVAALLHFFLLATFTWMGLEAIHMYIALVKVFNTYIHRYILKFCIIGWGLPALVVSIILVSRRQNEVYGKESYGKDQDDEFCWIQDPVVFYVSCAGYFGVMFFLNVAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHSDNFS

SEQ ID NO: 22 - DNA, 3495 bp, Mouse
atgatgtttgacactctcgggaagaggtgctgcccttggagactgaagccaagcgccctgctgttcctgtttgttttatgtgttacctgtgttcctctctcagtgtgcggatgtggcagctgcagacttgtcctgtccaatccttccggtacctttacgtctccgtgttaccctaatgactaccctaatacccagtcttgttcgtggaccctccgagcccctgccggctacatcattcagataacgttcaatgacttcgacattgaagaagctcccaactgtatctatgactcattgtccctcgataatggagagagccagacaaaattctgtggagcgactgccaagggcctgtcatttaactccagcgtgaatgagatgcatgtgtccttttcaagtgactttagtatccagaagaaaggtttcaacgccagctacatcagagttgctgtgtccttgaggaatcaaaaggtcattttgccccagacattagatgcttaccaggtatcagttgcaaaaagcatctccattcctgaactcaaagctttcacgctctgttttgaagcctccaaagttggcaatgaaggtggtgactggacagctttctcctactcagacgagtcccttacacagctgctcagtcttgaaaaggccagtaatggctacttcctgtccatctctggctcaagatgcttgttgaacaatgcgttacctgtgaaggacaaagaggacatcttcacagaaaacttggagcagctctgtcttgtgtggaataattcttggggctccgttggtataaatttcaaaaagaactatgaaacagttccatgcgattccaccatcagtgctgtcgtacccggggatgggacattgctgttgggctccgacagagatgagatcgcctctctaaggggcagcatctataactttcgactttggaattttaccatggatctgaaagccctctccaacctcagctgtagtgtgtctgggaatgtcatagactggcacaatgacttttggagcatctcaacccaagctctgaaagccgagggcaacctgagctgtggttcctacctgatccagcttcctgcagcagagctgacaaactgttcagaactggggactctctgtcaagacggaataatgtatcgaatatctgttgtgattcacaatgactttaatcaccctgaagtaaaagtgcagaccaaagtagcagaatggctcaattcaacctttcagaattggaactacactgtttatgtggttaatataagttttcatcaaaaagtaggagaggacaggatgaaagtcaagagagacatcatggacgatgacaaaaggttggtgctctgggcccttctagtctacaatgctaccaataacgtcagcctgaatgaagagaagattaaacaaaagcttatgacaaataatgcatcactagaggatggactgaggctgtgtgaagtcgacgtgaaccagctgggtatgtgtagcgccttggaggatccagacggctttagttggccagccaccttaccctctgtctacaaacaaccatgtccaaacaagcctggcttttttatgactcgagcgtgcctttctaatggaacatcaaccttctggggccctgttgacacttccaactgttcaagacaatcaaatgaagtggccaatgagattttaaaccaaactggtgatgggcagaacctcacctcggctaatatcaacagcattgtagaaaaggtcaaacggatcgtgaacaaagaagaaaacattgacatcaccctcggctccactctaatgaatatattttctaatatcttaagcagttcagatagcgatttgcttgagtcttctactgaagctttaaaaacaattgacgagctagccttcaaaatagacctgaatagcaccccacatgtgaacattgagacacagaatttggcccttggagtctcatccctaattccaggaacaaatgcaccttcaaattttagcattggccttccaagcaataatgaatcgtacttccagatggacttcgggaacggacagacagatccactggcatctgtgattttgcctccaaatttgcttgagaatttaagccccgaagattctgtattggtcaggagagcacagttcactttcttcaacaaaaccggacttttccaggatgttggatctcaaagaaaagtcctcgtgagttatgtgatggcgtgcagcattggaaacattactatccagaatctgaaagatccggttcaaatcaaaatcaaacacaccagaacacaggaagtgcatcatcctatctgtgccttctgggatatgaacaaaaacaaaagtttcggggggtggaacacctcaggatgtgttgcccactctgatttggacgctggtgagaccatttgtctgtgcagccacttcactcactttggagttctgatggatcttccaaggagtgcctcacaaatagatggaagaaacacaaaagtcctcacgttcattacctatattgggtgcggaatatctgccattttctcagctgcaactctcctgacatatgttgcttttgagaagctgcgcagggattatccctccaaaatcctgatgaatctgagctcggccttgctcttcctgaatctcatcttcctcctggatggctgggtcacttcctttggcgtggctggactctgcacggctgtggctgccctgttgcacttcttcctcctggctaccttcacctggatggggctggaagccatccacatgtacattgctcttgtgaaagtgtttaacacttacatccaccgctatattctaaaattctgcatcataggctggggtctgccagccttggtggtgtcaattattctagtgagcagaagacaaaatgaagtatatggaaaagaaagttatgggaaagatcaggatgatgaattctgctggattcaggatcctgtggtgttttatgtgagctgtgccgggtacttcggagtcatgttcttcctgaatgtcgccatgttcattgtggtcatggtgcagatctgtgggaggaatggaaagagaagcaaccggaccctgagagaagaggttttaagaaacctgcgcagtgtggtcagcctgaccttcctgcttggcatgacgtgggggtttgctttctttgcctggggacccttaaatattcctttcatgtacctcttctccatcttcaattcattacaaggtttatttatattcatcttccactgtgcgatgaaggagaatgttcagaaacagtggaggcgtcacctctgctgtggcaggtttcggctagcagacaactcagattggagtaagacagctaccaatatcatcaagaagagctccgataacctggggaaatctttgtcttcaagctccattggctccaattcaacatatctcacatccaaatcaaagtccagctccactacctatttcaaaagaaacagccactcggataatttctcc

SEQ ID NO: 23 - protein, 1220 aa, Mouse
MMFDTLGKRCCPWRLKPSALLFLFVLCVTCVPLSVCGCGSCRLVLSNPSGTFTSPCYPNDYPNTQSCSWTLRAPAGYIIQITFNDFDIEEAPNCIYDSLSLDNGESQTKFCGATAKGLSFNSSVNEMHVSFSSDFSIQKKGFNASYIRVAVSLRNQKVILPQTLDAYQVSVAKSISIPELKAFTLCFEASKVGNEGGDWTAFSYSDESLTQLLSLEKASNGYFLSISGSRCLLNNALPVKDKEDIFTENLEQLCLVWNNSWGSVGINFKKNYETVPCDSTISAVVPGDGTLLLGSDRDEIASLRGSIYNFRLWNFTMDLKALSNLSCSVSGNVIDWHNDFWSISTQALKAEGNLSCGSYLIQLPAAELTNCSELGTLCQDGIMYRISVVIHNDFNHPEVKVQTKVAEWLNSTFQNWNYTVYVVNISFHQKVGEDRMKVKRDIMDDDKRLVLWALLVYNATNNVSLNEEKIKQKLMTNNASLEDGLRLCEVDVNQLGMCSALEDPDGFSWPATLPSVYKQPCPNKPGFFMTRACLSNGTSTFWGPVDTSNCSRQSNEVANEILNQTGDGQNLTSANINSIVEKVKRIVNKEENIDITLGSTLMNIFSNILSSSDSDLLESSTEALKTIDELAFKIDLNSTPHVNIETQNLALGVSSLIPGTNAPSNFSIGLPSNNESYFQMDFGNGQTDPLASVILPPNLLENLSPEDSVLVRRAQFTFFNKTGLFQDVGSQRKVLVSYVMACSIGNITIQNLKDPVQIKIKHTRTQEVHHPICAFWDMNKNKSFGGWNTSGCVAHSDLDAGETICLCSHFTHFGVLMDLPRSASQIDGRNTKVLTFITYIGCGISAIFSAATLLTYVAFEKLRRDYPSKILMNLSSALLFLNLIFLLDGWVTSFGVAGLCTAVAALLHFFLLATFTWMGLEAIHMYIALVKVFNTYIHRYILKFCIIGWGLPALVVSIILVSRRQNEVYGKESYGKDQDDEFCWIQDPVVFYVSCAGYFGVMFFLNVAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHSDSTSTDKSLSKLTHAGGEQTSIIPVHQVIDKVKGYCNAHSDNFYKNIILSDAFSHSTKF

SEQ ID NO: 24 - DNA, 3660 bp, Mouse
atgatgtttgacactctcgggaagaggtgctgcccttggagactgaagccaagcgccctgctgttcctgtttgttttatgtgttacctgtgttcctctctcagtgtgcggatgtggcagctgcagacttgtcctgtccaatccttccggtacctttacgtctccgtgttaccctaatgactaccctaatacccagtcttgttcgtggaccctccgagcccctgccggctacatcattcagataacgttcaatgacttcgacattgaagaagctcccaactgtatctatgactcattgtccctcgataatggagagagccagacaaaattctgtggagcgactgccaagggcctgtcatttaactccagcgtgaatgagatgcatgtgtccttttcaagtgactttagtatccagaagaaaggtttcaacgccagctacatcagagttgctgtgtccttgaggaatcaaaaggtcattttgccccagacattagatgcttaccaggtatcagttgcaaaaagcatctccattcctgaactcaaagctttcacgctctgttttgaagcctccaaagttggcaatgaaggtggtgactggacagctttctcctactcagacgagtcccttacacagctgctcagtcttgaaaaggccagtaatggctacttcctgtccatctctggctcaagatgcttgttgaacaatgcgttacctgtgaaggacaaagaggacatcttcacagaaaacttggagcagctctgtcttgtgtggaataattcttggggctccgttggtataaatttcaaaaagaactatgaaacagttccatgcgattccaccatcagtgctgtcgtacccggggatgggacattgctgttgggctccgacagagatgagatcgcctctctaaggggcagcatctataactttcgactttggaattttaccatggatctgaaagccctctccaacctcagctgtagtgtgtctgggaatgtcatagactggcacaatgacttttggagcatctcaacccaagctctgaaagccgagggcaacctgagctgtggttcctacctgatccagcttcctgcagcagagctgacaaactgttcagaactggggactctctgtcaagacggaataatgtatcgaatatctgttgtgattcacaatgactttaatcaccctgaagtaaaagtgcagaccaaagtagcagaatggctcaattcaacctttcagaattggaactacactgtttatgtggttaatataagttttcatcaaaaagtaggagaggacaggatgaaagtcaagagagacatcatggacgatgacaaaaggttggtgctctgggcccttctagtctacaatgctaccaataacgtcagcctgaatgaagagaagattaaacaaaagcttatgacaaataatgcatcactagaggatggactgaggctgtgtgaagtcgacgtgaaccagctgggtatgtgtagcgccttggaggatccagacggctttagttggccagccaccttaccctctgtctacaaacaaccatgtccaaacaagcctggcttttttatgactcgagcgtgcctttctaatggaacatcaaccttctggggccctgttgacacttccaactgttcaagacaatcaaatgaagtggccaatgagattttaaaccaaactggtgatgggcagaacctcacctcggctaatatcaacagcattgtagaaaaggtcaaacggatcgtgaacaaagaagaaaacattgacatcaccctcggctccactctaatgaatatattttctaatatcttaagcagttcagatagcgatttgcttgagtcttctactgaagctttaaaaacaattgacgagctagccttcaaaatagacctgaatagcaccccacatgtgaacattgagacacagaatttggcccttggagtctcatccctaattccaggaacaaatgcaccttcaaattttagcattggccttccaagcaataatgaatcgtacttccagatggacttcgggaacggacagacagatccactggcatctgtgattttgcctccaaatttgcttgagaatttaagccccgaagattctgtattggtcaggagagcacagttcactttcttcaacaaaaccggacttttccaggatgttggatctcaaagaaaagtcctcgtgagttatgtgatggcgtgcagcattggaaacattactatccagaatctgaaagatccggttcaaatcaaaatcaaacacaccagaacacaggaagtgcatcatcctatctgtgccttctgggatatgaacaaaaacaaaagtttcggggggtggaacacctcaggatgtgttgcccactctgatttggacgctggtgagaccatttgtctgtgcagccacttcactcactttggagttctgatggatcttccaaggagtgcctcacaaatagatggaagaaacacaaaagtcctcacgttcattacctatattgggtgcggaatatctgccattttctcagctgcaactctcctgacatatgttgcttttgagaagctgcgcagggattatccctccaaaatcctgatgaatctgagctcggccttgctcttcctgaatctcatcttcctcctggatggctgggtcacttcctttggcgtggctggactctgcacggctgtggctgccctgttgcacttcttcctcctggctaccttcacctggatggggctggaagccatccacatgtacattgctcttgtgaaagtgtttaacacttacatccaccgctatattctaaaattctgcatcataggctggggtctgccagccttggtggtgtcaattattctagtgagcagaagacaaaatgaagtatatggaaaagaaagttatgggaaagatcaggatgatgaattctgctggattcaggatcctgtggtgttttatgtgagctgtgccgggtacttcggagtcatgttcttcctgaatgtcgccatgttcattgtggtcatggtgcagatctgtgggaggaatggaaagagaagcaaccggaccctgagagaagaggttttaagaaacctgcgcagtgtggtcagcctgaccttcctgcttggcatgacgtgggggtttgctttctttgcctggggacccttaaatattcctttcatgtacctcttctccatcttcaattcattacaaggtttatttatattcatcttccactgtgcgatgaaggagaatgttcagaaacagtggaggcgtcacctctgctgtggcaggtttcggctagcagacaactcagattggagtaagacagctaccaatatcatcaagaagagctccgataacctggggaaatctttgtcttcaagctccattggctccaattcaacatatctcacatccaaatcaaagtccagctccactacctatttcaaaagaaacagccactcggacagcacttccacggacaagtccctgtcaaaactgacccatgctggtggagaacagacgtcaatcattcccgtccatcaggttattgataaggtcaagggttactgtaatgcccattccgataacttctataaaaatatcatcctgtcagatgccttcagccacagcacaaagttt

SEQ ID NO: 25 - protein, 1175 aa, Mouse
MSVRPLGISTRMFDTLGKRCCPWRLKPSALLFLFVLCVTCVPLSVCGCGSCRLVLSNPSGTFTSPCYPNDYPNTQSCSWTLRAPAGYIIQITFNDFDIEEAPNCIYDSLSLDNGESQTKFCGATAKGLSFNSSVNEMHVSFSSDFSIQKKGFNASYIRVAVSLRNQKVILPQTLDAYQVSVAKSISIPELKAFTLCFEASKVGNEGGDWTAFSYSDESLTQLLSLEKASNGYFLSISGSRCLLNNALPVKDKEDIFTENLEQLCLVWNNSWGSVGINFKKNYETVPCDSTISAVVPGDGTLLLGSDRDEIASLRGSIYNFRLWNFTMDLKALSNLSCSVSGNVIDWHNDFWSISTQALKAEGNLSCGSYLIQLPAAELTNCSELGTLCQDGIMYRISVVIHNDFNHPEVKVQTKVAEWLNSTFQNWNYTVYVVNISFHQKVGEDRMKVKRDIMDDDKRLVLWALLVYNATNNVSLNEEKIKQKLMTNNASLEDGLRLCEVDVNQLGMCSALEDPDGFSWPATLPSVYKQPCPNKPGFFMTRACLSNGTSTFWGPVDTSNCSRQSNEVANEILNQTGDGQNLTSANINSIVEKVKRIVNKEENIDITLGSTLMNIFSNILSSSDSDLLESSTEALKTIDELAFKIDLNSTPHVNIETQNLALGVSSLIPGTNAPSNFSIGLPSNNESYFQMDFGNGQTDPLASVILPPNLLENLSPEDSVLVRRAQFTFFNKTGLFQDVGSQRKVLVSYVMACSIGNITIQNLKDPVQIKIKHTRTQEVHHPICAFWDMNKNKSFGGWNTSGCVAHSDLDAGETICLCSHFTHFGVLMDLPRSASQIDGRNTKVLTFITYIGCGISAIFSAATLLTYVAFEKLRRDYPSKILMNLSSALLFLNLIFLLDGWVTSFGVAGLCTAVAALLHFFLLATFTWMGLEAIHMYIALVKVFNTYIHRYILKFCIIGWGLPALVVSIILVSRRQNEVYGKESYGKDQDDEFCWIQDPVVFYVSCAGYFGVMFFLNVAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHSDNFS

SEQ ID NO: 26 - DNA, 3525 bp, Mouse
atgagtgtgcggcctctgggaatctccacacggatgtttgacactctcgggaagaggtgctgcccttggagactgaagccaagcgccctgctgttcctgtttgttttatgtgttacctgtgttcctctctcagtgtgcggatgtggcagctgcagacttgtcctgtccaatccttccggtacctttacgtctccgtgttaccctaatgactaccctaatacccagtcttgttcgtggaccctccgagcccctgccggctacatcattcagataacgttcaatgacttcgacattgaagaagctcccaactgtatctatgactcattgtccctcgataatggagagagccagacaaaattctgtggagcgactgccaagggcctgtcatttaactccagcgtgaatgagatgcatgtgtccttttcaagtgactttagtatccagaagaaaggtttcaacgccagctacatcagagttgctgtgtccttgaggaatcaaaaggtcattttgccccagacattagatgcttaccaggtatcagttgcaaaaagcatctccattcctgaactcaaagctttcacgctctgttttgaagcctccaaagttggcaatgaaggtggtgactggacagctttctcctactcagacgagtcccttacacagctgctcagtcttgaaaaggccagtaatggctacttcctgtccatctctggctcaagatgcttgttgaacaatgcgttacctgtgaaggacaaagaggacatcttcacagaaaacttggagcagctctgtcttgtgtggaataattcttggggctccgttggtataaatttcaaaaagaactatgaaacagttccatgcgattccaccatcagtgctgtcgtacccggggatgggacattgctgttgggctccgacagagatgagatcgcctctctaaggggcagcatctataactttcgactttggaattttaccatggatctgaaagccctctccaacctcagctgtagtgtgtctgggaatgtcatagactggcacaatgacttttggagcatctcaacccaagctctgaaagccgagggcaacctgagctgtggttcctacctgatccagcttcctgcagcagagctgacaaactgttcagaactggggactctctgtcaagacggaataatgtatcgaatatctgttgtgattcacaatgactttaatcaccctgaagtaaaagtgcagaccaaagtagcagaatggctcaattcaacctttcagaattggaactacactgtttatgtggttaatataagttttcatcaaaaagtaggagaggacaggatgaaagtcaagagagacatcatggacgatgacaaaaggttggtgctctgggcccttctagtctacaatgctaccaataacgtcagcctgaatgaagagaagattaaacaaaagcttatgacaaataatgcatcactagaggatggactgaggctgtgtgaagtcgacgtgaaccagctgggtatgtgtagcgccttggaggatccagacggctttagttggccagccaccttaccctctgtctacaaacaaccatgtccaaacaagcctggcttttttatgactcgagcgtgcctttctaatggaacatcaaccttctggggccctgttgacacttccaactgttcaagacaatcaaatgaagtggccaatgagattttaaaccaaactggtgatgggcagaacctcacctcggctaatatcaacagcattgtagaaaaggtcaaacggatcgtgaacaaagaagaaaacattgacatcaccctcggctccactctaatgaatatattttctaatatcttaagcagttcagatagcgatttgcttgagtcttctactgaagctttaaaaacaattgacgagctagccttcaaaatagacctgaatagcaccccacatgtgaacattgagacacagaatttggcccttggagtctcatccctaattccaggaacaaatgcaccttcaaattttagcattggccttccaagcaataatgaatcgtacttccagatggacttcgggaacggacagacagatccactggcatctgtgattttgcctccaaatttgcttgagaatttaagccccgaagattctgtattggtcaggagagcacagttcactttcttcaacaaaaccggacttttccaggatgttggatctcaaagaaaagtcctcgtgagttatgtgatggcgtgcagcattggaaacattactatccagaatctgaaagatccggttcaaatcaaaatcaaacacaccagaacacaggaagtgcatcatcctatctgtgccttctgggatatgaacaaaaacaaaagtttcggggggtggaacacctcaggatgtgttgcccactctgatttggacgctggtgagaccatttgtctgtgcagccacttcactcactttggagttctgatggatcttccaaggagtgcctcacaaatagatggaagaaacacaaaagtcctcacgttcattacctatattgggtgcggaatatctgccattttctcagctgcaactctcctgacatatgttgcttttgagaagctgcgcagggattatccctccaaaatcctgatgaatctgagctcggccttgctcttcctgaatctcatcttcctcctggatggctgggtcacttcctttggcgtggctggactctgcacggctgtggctgccctgttgcacttcttcctcctggctaccttcacctggatggggctggaagccatccacatgtacattgctcttgtgaaagtgtttaacacttacatccaccgctatattctaaaattctgcatcataggctggggtctgccagccttggtggtgtcaattattctagtgagcagaagacaaaatgaagtatatggaaaagaaagttatgggaaagatcaggatgatgaattctgctggattcaggatcctgtggtgttttatgtgagctgtgccgggtacttcggagtcatgttcttcctgaatgtcgccatgttcattgtggtcatggtgcagatctgtgggaggaatggaaagagaagcaaccggaccctgagagaagaggttttaagaaacctgcgcagtgtggtcagcctgaccttcctgcttggcatgacgtgggggtttgctttctttgcctggggacccttaaatattcctttcatgtacctcttctccatcttcaattcattacaaggtttatttatattcatcttccactgtgcgatgaaggagaatgttcagaaacagtggaggcgtcacctctgctgtggcaggtttcggctagcagacaactcagattggagtaagacagctaccaatatcatcaagaagagctccgataacctggggaaatctttgtcttcaagctccattggctccaattcaacatatctcacatccaaatcaaagtccagctccactacctatttcaaaagaaacagccactcggataatttctcc

SEQ ID NO: 27 - protein, 1230 aa, Mouse
MSVRPLGISTRMFDTLGKRCCPWRLKPSALLFLFVLCVTCVPLSVCGCGSCRLVLSNPSGTFTSPCYPNDYPNTQSCSWTLRAPAGYIIQITFNDFDIEEAPNCIYDSLSLDNGESQTKFCGATAKGLSFNSSVNEMHVSFSSDFSIQKKGFNASYIRVAVSLRNQKVILPQTLDAYQVSVAKSISIPELKAFTLCFEASKVGNEGGDWTAFSYSDESLTQLLSLEKASNGYFLSISGSRCLLNNALPVKDKEDIFTENLEQLCLVWNNSWGSVGINFKKNYETVPCDSTISAVVPGDGTLLLGSDRDEIASLRGSIYNFRLWNFTMDLKALSNLSCSVSGNVIDWHNDFWSISTQALKAEGNLSCGSYLIQLPAAELTNCSELGTLCQDGIMYRISVVIHNDFNHPEVKVQTKVAEWLNSTFQNWNYTVYVVNISFHQKVGEDRMKVKRDIMDDDKRLVLWALLVYNATNNVSLNEEKIKQKLMTNNASLEDGLRLCEVDVNQLGMCSALEDPDGFSWPATLPSVYKQPCPNKPGFFMTRACLSNGTSTFWGPVDTSNCSRQSNEVANEILNQTGDGQNLTSANINSIVEKVKRIVNKEENIDITLGSTLMNIFSNILSSSDSDLLESSTEALKTIDELAFKIDLNSTPHVNIETQNLALGVSSLIPGTNAPSNFSIGLPSNNESYFQMDFGNGQTDPLASVILPPNLLENLSPEDSVLVRRAQFTFFNKTGLFQDVGSQRKVLVSYVMACSIGNITIQNLKDPVQIKIKHTRTQEVHHPICAFWDMNKNKSFGGWNTSGCVAHSDLDAGETICLCSHFTHFGVLMDLPRSASQIDGRNTKVLTFITYIGCGISAIFSAATLLTYVAFEKLRRDYPSKILMNLSSALLFLNLIFLLDGWVTSFGVAGLCTAVAALLHFFLLATFTWMGLEAIHMYIALVKVFNTYIHRYILKFCIIGWGLPALVVSIILVSRRQNEVYGKESYGKDQDDEFCWIQDPVVFYVSCAGYFGVMFFLNVAMFIVVMVQICGRNGKRSNRTLREEVLRNLRSVVSLTFLLGMTWGFAFFAWGPLNIPFMYLFSIFNSLQGLFIFIFHCAMKENVQKQWRRHLCCGRFRLADNSDWSKTATNIIKKSSDNLGKSLSSSSIGSNSTYLTSKSKSSSTTYFKRNSHSDSTSTDKSLSKLTHAGGEQTSIIPVHQVIDKVKGYCNAHSDNFYKNIILSDAFSHSTKF

SEQ ID NO: 28 - DNA, 3690 bp, Mouse
atgagtgtgcggcctctgggaatctccacacggatgtttgacactctcgggaagaggtgctgcccttggagactgaagccaagcgccctgctgttcctgtttgttttatgtgttacctgtgttcctctctcagtgtgcggatgtggcagctgcagacttgtcctgtccaatccttccggtacctttacgtctccgtgttaccctaatgactaccctaatacccagtcttgttcgtggaccctccgagcccctgccggctacatcattcagataacgttcaatgacttcgacattgaagaagctcccaactgtatctatgactcattgtccctcgataatggagagagccagacaaaattctgtggagcgactgccaagggcctgtcatttaactccagcgtgaatgagatgcatgtgtccttttcaagtgactttagtatccagaagaaaggtttcaacgccagctacatcagagttgctgtgtccttgaggaatcaaaaggtcattttgccccagacattagatgcttaccaggtatcagttgcaaaaagcatctccattcctgaactcaaagctttcacgctctgttttgaagcctccaaagttggcaatgaaggtggtgactggacagctttctcctactcagacgagtcccttacacagctgctcagtcttgaaaaggccagtaatggctacttcctgtccatctctggctcaagatgcttgttgaacaatgcgttacctgtgaaggacaaagaggacatcttcacagaaaacttggagcagctctgtcttgtgtggaataattcttggggctccgttggtataaatttcaaaaagaactatgaaacagttccatgcgattccaccatcagtgctgtcgtacccggggatgggacattgctgttgggctccgacagagatgagatcgcctctctaaggggcagcatctataactttcgactttggaattttaccatggatctgaaagccctctccaacctcagctgtagtgtgtctgggaatgtcatagactggcacaatgacttttggagcatctcaacccaagctctgaaagccgagggcaacctgagctgtggttcctacctgatccagcttcctgcagcagagctgacaaactgttcagaactggggactctctgtcaagacggaataatgtatcgaatatctgttgtgattcacaatgactttaatcaccctgaagtaaaagtgcagaccaaagtagcagaatggctcaattcaacctttcagaattggaactacactgtttatgtggttaatataagttttcatcaaaaagtaggagaggacaggatgaaagtcaagagagacatcatggacgatgacaaaaggttggtgctctgggcccttctagtctacaatgctaccaataacgtcagcctgaatgaagagaagattaaacaaaagcttatgacaaataatgcatcactagaggatggactgaggctgtgtgaagtcgacgtgaaccagctgggtatgtgtagcgccttggaggatccagacggctttagttggccagccaccttaccctctgtctacaaacaaccatgtccaaacaagcctggcttttttatgactcgagcgtgcctttctaatggaacatcaaccttctggggccctgttgacacttccaactgttcaagacaatcaaatgaagtggccaatgagattttaaaccaaactggtgatgggcagaacctcacctcggctaatatcaacagcattgtagaaaaggtcaaacggatcgtgaacaaagaagaaaacattgacatcaccctcggctccactctaatgaatatattttctaatatcttaagcagttcagatagcgatttgcttgagtcttctactgaagctttaaaaacaattgacgagctagccttcaaaatagacctgaatagcaccccacatgtgaacattgagacacagaatttggcccttggagtctcatccctaattccaggaacaaatgcaccttcaaattttagcattggccttccaagcaataatgaatcgtacttccagatggacttcgggaacggacagacagatccactggcatctgtgattttgcctccaaatttgcttgagaatttaagccccgaagattctgtattggtcaggagagcacagttcactttcttcaacaaaaccggacttttccaggatgttggatctcaaagaaaagtcctcgtgagttatgtgatggcgtgcagcattggaaacattactatccagaatctgaaagatccggttcaaatcaaaatcaaacacaccagaacacaggaagtgcatcatcctatctgtgccttctgggatatgaacaaaaacaaaagtttcggggggtggaacacctcaggatgtgttgcccactctgatttggacgctggtgagaccatttgtctgtgcagccacttcactcactttggagttctgatggatcttccaaggagtgcctcacaaatagatggaagaaacacaaaagtcctcacgttcattacctatattgggtgcggaatatctgccattttctcagctgcaactctcctgacatatgttgcttttgagaagctgcgcagggattatccctccaaaatcctgatgaatctgagctcggccttgctcttcctgaatctcatcttcctcctggatggctgggtcacttcctttggcgtggctggactctgcacggctgtggctgccctgttgcacttcttcctcctggctaccttcacctggatggggctggaagccatccacatgtacattgctcttgtgaaagtgtttaacacttacatccaccgctatattctaaaattctgcatcataggctggggtctgccagccttggtggtgtcaattattctagtgagcagaagacaaaatgaagtatatggaaaagaaagttatgggaaagatcaggatgatgaattctgctggattcaggatcctgtggtgttttatgtgagctgtgccgggtacttcggagtcatgttcttcctgaatgtcgccatgttcattgtggtcatggtgcagatctgtgggaggaatggaaagagaagcaaccggaccctgagagaagaggttttaagaaacctgcgcagtgtggtcagcctgaccttcctgcttggcatgacgtgggggtttgctttctttgcctggggacccttaaatattcctttcatgtacctcttctccatcttcaattcattacaaggtttatttatattcatcttccactgtgcgatgaaggagaatgttcagaaacagtggaggcgtcacctctgctgtggcaggtttcggctagcagacaactcagattggagtaagacagctaccaatatcatcaagaagagctccgataacctggggaaatctttgtcttcaagctccattggctccaattcaacatatctcacatccaaatcaaagtccagctccactacctatttcaaaagaaacagccactcggacagcacttccacggacaagtccctgtcaaaactgacccatgctggtggagaacagacgtcaatcattcccgtccatcaggttattgataaggtcaagggttactgtaatgcccattccgataacttctataaaaatatcatcctgtcagatgccttcagccacagcacaaagttt

SEQ ID NO: 29 - DNA, 27 bp, Artificial Sequence
  Primer
gaagaggtgctgcccttggagactgaa

SEQ ID NO: 30 - DNA, 27 bp, Artificial Sequence
  Primer
ctcttaggagaaattatccgagtggct

SEQ ID NO: 31 - DNA, 24 bp, Artificial Sequence
  Primer
ggacattgctgttgggctccgaca

SEQ ID NO: 32 - DNA, 24 bp, Artificial Sequence
  Primer
tgcacgccatcacataactcacga

SEQ ID NO: 33 - DNA, 24 bp, Artificial Sequence
  Primer
gcaggtttcggctagcagacaact

SEQ ID NO: 34 - DNA, 26 bp, Artificial Sequence
  Primer
ccaaatcaaagtccagctccactacc

SEQ ID NO: 35 - DNA, 21 bp, Artificial Sequence
  Primer
aaggtaccggaaggattggac

SEQ ID NO: 36 - DNA, 21 bp, Artificial Sequence
  Primer
cattagggtaacacggagacg

SEQ ID NO: 37 - DNA, 21 bp, Artificial Sequence
  Primer
cggagggtccacgaacaagac

SEQ ID NO: 38 - protein, 382 aa, human
MGPTSVPLVKAHRSSVSDYVNYDIIVRHYNYTGKLNISADKENSIKLTSVVFILICCFIILENIFVLLTIWKTKKFHRPMYYFIGNLALSDLLAGVAYTANLLLSGATTYKLTPAQWFLREGSMFVALSASVFSLLAIAIERYITMLKMKLHNGSNNFRLFLLISACWVISLILGGLPIMGWNCISALSSCSTVLPLYHKHYILFCTTVFTLLLLSIVILYCRIYSLVRTRSRRLTFRKNISKASRSSEKSLALLKTVIIVLSVFIACWAPLFILLLLDVGCKVKTCDILFRAEYFLVLAVLNSGTNPIIYTLTNKEMRRAFIRIMSCCKCPSGDSAGKFKRPIIAGMEFSRSKSDNSSHPQKDEGDNPETIMSSGNVNSSS

SEQ ID NO: 39 - DNA, 1146 bp, human
atggggcccaccagcgtcccgctggtcaaggcccaccgcagctcggtctctgactacgtcaactatgatatcatcgtccggcattacaactacacgggaaagctgaatatcagcgcggacaaggagaacagcattaaactgacctcggtggtgttcattctcatctgctgctttatcatcctggagaacatctttgtcttgctgaccatttggaaaaccaagaaattccaccgacccatgtactattttattggcaatctggccctctcagacctgttggcaggagtagcctacacagctaacctgctcttgtctggggccaccacctacaagctcactcccgcccagtggtttctgcgggaagggagtatgtttgtggccctgtcagcctccgtgttcagtctcctcgccatcgccattgagcgctatatcacaatgctgaaaatgaaactccacaacgggagcaataacttccgcctcttcctgctaatcagcgcctgctgggtcatctccctcatcctgggtggcctgcctatcatgggctggaactgcatcagtgcgctgtccagctgctccaccgtgctgccgctctaccacaagcactatatcctcttctgcaccacggtcttcactctgcttctgctctccatcgtcattctgtactgcagaatctactccttggtcaggactcggagccgccgcctgacgttccgcaagaacatttccaaggccagccgcagctctgagaagtcgctggcgctgctcaagaccgtaattatcgtcctgagcgtcttcatcgcctgctgggcaccgctcttcatcctgctcctgctggatgtgggctgcaaggtgaagacctgtgacatcctcttcagagcggagtacttcctggtgttagctgtgctcaactccggcaccaaccccatcatttacactctgaccaacaaggagatgcgtcgggccttcatccggatcatgtcctgctgcaagtgcccgagcggagactctgctggcaaattcaagcgacccatcatcgccggcatggaattcagccgcagcaaatcggacaattcctcccacccccagaaagacgaaggggacaacccagagaccattatgtcttctggaaacgtcaactcttcttcc

SEQ ID NO: 40 - protein, 364 aa, human
MAAISTSIPVISQPQFTAMNEPQCFYNESIAFFYNRSGKHLATEWNTVSKLVMGLGITVCIFIMLANLLVMVAIYVNRRFHFPIYYLMANLAAADFFAGLAYFYLMFNTGPNTRRLTVSTWLLRQGLIDTSLTASVANLLAIAIERHITVFRMQLHTRMSNRRVVVVIVVIWTMAIVMGAIPSVGWNCICDIENCSNMAPLYSDSYLVFWAIFNLVTFVVMVVLYAHIFGYVRQRTMRMSRHSSGPRRNRDTMMSLLKTVVIVLGAFIICWTPGLVLLLLDVCCPQCDVLAYEKFFLLLAEFNSAMNPIIYSYRDKEMSATFRQILCCQRSENPTGPTEGSDRSASSLNHTILAGVHSNDHSVV

SEQ ID NO: 41 - DNA, 1092 bp, human
atggctgccatctctacttccatccctgtaatttcacagccccagttcacagccatgaatgaaccacagtgcttctacaacgagtccattgccttcttttataaccgaagtggaaagcatcttgccacagaatggaacacagtcagcaagctggtgatgggacttggaatcactgtttgtatcttcatcatgttggccaacctattggtcatggtggcaatctatgtcaaccgccgcttccattttcctatttattacctaatggctaatctggctgctgcagacttctttgctgggttggcctacttctatctcatgttcaacacaggacccaatactcggagactgactgttagcacatggctcctgcgtcagggcctcattgacaccagcctgacggcatctgtggccaacttactggctattgcaatcgagaggcacattacggttttccgcatgcagctccacacacggatgagcaaccggcgggtagtggtggtcattgtggtcatctggactatggccatcgttatgggtgctatacccagtgtgggctggaactgtatctgtgatattgaaaattgttccaacatggcacccctctacagtgactcttacttagtcttctgggccattttcaacttggtgacctttgtggtaatggtggttctctatgctcacatctttggctatgttcgccagaggactatgagaatgtctcggcatagttctggaccccggcggaatcgggataccatgatgagtcttctgaagactgtggtcattgtgcttggggcctttatcatctgctggactcctggattggttttgttacttctagacgtgtgctgtccacagtgcgacgtgctggcctatgagaaattcttccttctccttgctgaattcaactctgccatgaaccccatcatttactcctaccgcgacaaagaaatgagcgccacctttaggcagatcctctgctgccagcgcagtgagaaccccaccggccccacagaaggctcagaccgctcggcttcctccctcaaccacaccatcttggctggagttcacagcaatgaccactctgtggtt

SEQ ID NO: 42 - protein, 378 aa, human
MATALPPRLQPVRGNETLREHYQYVGKLAGRLKEASEGSTLTTVLFLVICSFIVLENLMVLIAIWKNNKFHNRMYFFIGNLALCDLLAGIAYKVNILMSGKKTFSLSPTVWFLREGSMFVALGASTCSLLAIAIERHLTMIKMRPYDANKRHRVFLLIGMCWLIAFTLGALPILGWNCLHNLPDCSTILPLYSKKYIAFCISIFTAILVTIVILYARIYFLVKSSSRKVANHNNSERSMALLRTVVIVVSVFIACWSPLFILFLIDVACRVQACPILFKAQWFIVLAVLNSAMNPVIYTLASKEMRRAFFRLVCNCLVRGRGARASPIQPALDPSRSKSSSSNNSSHSPKVKEDLPHTDPSSCIMDKNAALQNGIFCN

SEQ ID NO: 43 - DNA, 1134 bp, human
atggcaactgccctcccgccgcgtctccagccggtgcgggggaacgagaccctgcgggagcattaccagtacgtggggaagttggcgggcaggctgaaggaggcctccgagggcagcacgctcaccaccgtgctcttcttggtcatctgcagcttcatcgtcttggagaacctgatggttttgattgccatctggaaaaacaataaatttcacaaccgcatgtactttttcattggcaacctggctctctgcgacctgctggccggcatcgcttacaaggtcaacattctgatgtctggcaagaagacgttcagcctgtctcccacggtctggttcctcagggagggcagtatgttcgtggcccttggggcgtccacctgcagcttactggccatcgccatcgagcggcacttgacaatgatcaaaatgaggccttacgacgccaacaagaggcaccgcgtcttcctcctgatcgggatgtgctggctcattgccttcacgctgggcgccctgcccattctgggctggaactgcctgcacaatctccctgactgctctaccatcctgcccctctactccaagaagtacattgccttctgcatcagcatcttcacggccatcctggtgaccatcgtgatcctctacgcacgcatctacttcctggtgaagtccagcagccgtaaggtggccaaccacaacaactcggagcggtccatggcactgctgcggaccgtggtgattgtggtgagcgtgttcatcgcctgctggtccccactcttcatcctcttcctcattgatgtggcctgcagggtgcaggcgtgccccatcctcttcaaggctcagtggttcatcgtgttggctgtgctcaactccgccatgaacccggtcatctacacgctggccagcaaggagatgcggcgggccttcttccgtctggtctgcaactgcctggtcaggggacggggggcccgcgcctcacccatccagcctgcgctcgacccaagcagaagtaaatcaagcagcagcaacaatagcagccactctccgaaggtcaaggaagacctgccccacacagacccctcatcctgcatcatggacaagaacgcagcacttcagaatgggatcttctgcaac

SEQ ID NO: 44 - protein, 351 aa, human
MVIMGQCYYNETIGFFYNNSGKELSSHWRPKDVVVVALGLTVSVLVLLTNLLVIAAIASNRRFHQPIYYLLGNLAAADLFAGVAYLFLMFHTGPRTARLSLEGWFLRQGLLDTSLTASVATLLAIAVERHRSVMAVQLHSRLPRGRVVMLIVGVWVAALGLGLLPAHSWHCLCALDRCSRMAPLLSRSYLAVWALSSLLVFLLMVAVYTRIFFYVRRRVQRMAEHVSCHPRYRETTLSLVKTVVIILGAFVVCWTPGQVVLLLDGLGCESCNVLAVEKYFLLLAEANSLVNAAVYSCRDAEMRRTFRRLLCCACLRQSTRESVHYTSSAQGGASTRIMLPENGHPLMDSTL

SEQ ID NO: 45 - DNA, 1056 bp, human
atggtcatcatgggccagtgctactacaacgagaccatcggcttcttctataacaacagtggcaaagagctcagctcccactggcggcccaaggatgtggtcgtggtggcactggggctgaccgtcagcgtgctggtgctgctgaccaatctgctggtcatagcagccatcgcctccaaccgccgcttccaccagcccatctactacctgctcggcaatctggccgcggctgacctcttcgcgggcgtggcctacctcttcctcatgttccacactggtccccgcacagcccgactttcacttgagggctggttcctgcggcagggcttgctggacacaagcctcactgcgtcggtggccacactgctggccatcgccgtggagcggcaccgcagtgtgatggccgtgcagctgcacagccgcctgccccgtggccgcgtggtcatgctcattgtgggcgtgtgggtggctgccctgggcctggggctgctgcctgcccactcctggcactgcctctgtgccctggaccgctgctcacgcatggcacccctgctcagccgctcctatttggccgtctgggctctgtcgagcctgcttgtcttcctgctcatggtggctgtgtacacccgcattttcttctacgtgcggcggcgagtgcagcgcatggcagagcatgtcagctgccacccccgctaccgagagaccacgctcagcctggtcaagactgttgtcatcatcctgggggcgttcgtggtctgctggacaccaggccaggtggtactgctcctggatggtttaggctgtgagtcctgcaatgtcctggctgtagaaaagtacttcctactgttggccgaggccaactcactggtcaatgctgctgtgtactcttgccgagatgctgagatgcgccgcaccttccgccgccttctctgctgcgcgtgcctccgccagtccacccgcgagtctgtccactatacatcctctgcccagggaggtgccagcactcgcatcatgcttcccgagaacggccacccactgatggactccaccctttag

SEQ ID NO: 46 - protein, 353 aa, human
MGSLYSEYLNPNKVQEHYNYTKETLETQETTSRQVASAFIVILCCAIVVENLLVLIAVARNSKFHSAMYLFLGNLAASDLLAGVAFVANTLLSGSVTLRLTPVQWFAREGSASITLSASVFSLLAIAIERHVAIAKVKLYGSDKSCRMLLLIGASWLISLVLGGLPILGWNCLGHLEACSTVLPLYAKHYVLCVVTIFSIILLAIVALYVRIYCVVRSSHADMAAPQTLALLKTVTIVLGVFIVCWLPAFSILLLDYACPVHSCPILYKAHYFFAVSTLNSLLNPVIYTWRSRDLRREVLRPLQCWRPGVGVQGRRRVGTPGHHLLPLRSSSSLERGMHMPTSPTFLEGNTVV

SEQ ID NO: 47 - DNA, 1059 bp, human
atgggcagcttgtactcggagtacctgaaccccaacaaggtccaggaacactataattataccaaggagacgctggaaacgcaggagacgacctcccgccaggtggcctcggccttcatcgtcatcctctgttgcgccattgtggtggaaaaccttctggtgctcattgcggtggcccgaaacagcaagttccactcggcaatgtacctgtttctgggcaacctggccgcctccgatctactggcaggcgtggccttcgtagccaataccttgctctctggctctgtcacgctgaggctgacgcctgtgcagtggtttgcccgggagggctctgcctccatcacgctctcggcctctgtcttcagcctcctggccatcgccattgagcgccacgtggccattgccaaggtcaagctgtatggcagcgacaagagctgccgcatgcttctgctcatcggggcctcgtggctcatctcgctggtcctcggtggcctgcccatccttggctggaactgcctgggccacctcgaggcctgctccactgtcctgcctctctacgccaagcattatgtgctgtgcgtggtgaccatcttctccatcatcctgttggccatcgtggccctgtacgtgcgcatctactgcgtggtccgctcaagccacgctgacatggccgccccgcagacgctagccctgctcaagacggtcaccatcgtgctaggcgtctttatcgtctgctggctgcccgccttcagcatcctccttctggactatgcctgtcccgtccactcctgcccgatcctctacaaagcccactactttttcgccgtctccaccctgaattccctgctcaaccccgtcatctacacgtggcgcagccgggacctgcggcgggaggtgcttcggccgctgcagtgctggcggccgggggtgggggtgcaaggacggaggcgggtcgggaccccgggccaccacctcctgccactccgcagctccagctccctggagaggggcatgcacatgcccacgtcacccacgtttctggagggcaacacggtggtc

SEQ ID NO: 48 - protein, 384 aa, human
MNATGTPVAPESCQQLAAGGHSRLIVLHYNHSGRLAGRGGPEDGGLGALRGLSVAASCLVVLENLLVLAAITSHMRSRRWVYYCLVNITLSDLLTGAAYLANVLLSGARTFRLAPAQWFLREGLLFTALAASTFSLLFTAGERFATMVRPVAESGATKTSRVYGFIGLCWLLAALLGMLPLLGWNCLCAFDRCSSLLPLYSKRYILFCLVIFAGVLATIMGLYGAIFRLVQASGQKAPRPAARRKARRLLKTVLMILLAFLVCWGPLFGLLLADVFGSNLWAQEYLRGMDWILALAVLNSAVNPIIYSFRSREVCRAVLSFLCCGCLRLGMRGPGDCLARAVEAHSGASTTDSSLRPRDSFRGSRSLSFRMREPLSSISSVRSI

SEQ ID NO: 49 - DNA, 1152 bp, human
atgaacgccacggggaccccggtggcccccgagtcctgccaacagctggcggccggcgggcacagccggctcattgttctgcactacaaccactcgggccggctggccgggcgcggggggccggaggatggcggcctgggggccctgcgggggctgtcggtggccgccagctgcctggtggtgctggagaacttgctggtgctggcggccatcaccagccacatgcggtcgcgacgctgggtctactattgcctggtgaacatcacgctgagtgacctgctcacgggcgcggcctacctggccaacgtgctgctgtcgggggcccgcaccttccgtctggcgcccgcccagtggttcctacgggagggcctgctcttcaccgccctggccgcctccaccttcagcctgctcttcactgcaggggagcgctttgccaccatggtgcggccggtggccgagagcggggccaccaagaccagccgcgtctacggcttcatcggcctctgctggctgctggccgcgctgctggggatgctgcctttgctgggctggaactgcctgtgcgcctttgaccgctgctccagccttctgcccctctactccaagcgctacatcctcttctgcctggtgatcttcgccggcgtcctggccaccatcatgggcctctatggggccatcttccgcctggtgcaggccagcgggcagaaggccccacgcccagcggcccgccgcaaggcccgccgcctgctgaagacggtgctgatgatcctgctggccttcctggtgtgctggggcccactcttcgggctgctgctggccgacgtctttggctccaacctctgggcccaggagtacctgcggggcatggactggatcctggccctggccgtcctcaactcggcggtcaaccccatcatctactccttccgcagcagggaggtgtgcagagccgtgctcagcttcctctgctgcgggtgtctccggctgggcatgcgagggcccggggactgcctggcccgggccgtcgaggctcactccggagcttccaccaccgacagctctctgaggccaagggacagctttcgcggctcccgctcgctcagctttcggatgcgggagcccctgtccagcatctccagcgtgcggagcatc

SEQ ID NO: 50 - protein, 353 aa, human
MNECHYDKHMDFFYNRSNTDTVDDWTGTKLVIVLCVGTFFCLFIFFSNSLVIAAVIKNRKFHFPFYYLLANLAAADFFAGIAYVFLMFNTGPVSKTLTVNRWFLRQGLLDSSLTASLTNLLVIAVERHMSIMRMRVHSNLTKKRVTLLILLVWAIAIFMGAVPTLGWNCLCNISACSSLAPIYSRSYLVFWTVSNLMAFLIMVVVYLRIYVYVKRKTNVLSPHTSGSISRRRTPMKLMKTVMTVLGAFVVCWTPGLVVLLLDGLNCRQCGVQHVKRWFLLLALLNSVVNPIIYSYKDEDMYGTMKKMICCFSQENPERRPSRIPSTVLSRSDTGSQYIEDSISQGAVCNKSTS

SEQ ID NO: 51 - DNA, 1059 bp, human
atgaatgagtgtcactatgacaagcacatggactttttttataataggagcaacactgatactgtcgatgactggacaggaacaaagcttgtgattgttttgtgtgttgggacgtttttctgcctgtttatttttttttctaattctctggtcatcgcggcagtgatcaaaaacagaaaatttcatttccccttctactacctgttggctaatttagctgctgccgatttcttcgctggaattgcctatgtattcctgatgtttaacacaggcccagtttcaaaaactttgactgtcaaccgctggtttctccgtcaggggcttctggacagtagcttgactgcttccctcaccaacttgctggttatcgccgtggagaggcacatgtcaatcatgaggatgcgggtccatagcaacctgaccaaaaagagggtgacactgctcattttgcttgtctgggccatcgccatttttatgggggcggtccccacactgggctggaattgcctctgcaacatctctgcctgctcttccctggcccccatttacagcaggagttaccttgttttctggacagtgtccaacctcatggccttcctcatcatggttgtggtgtacctgcggatctacgtgtacgtcaagaggaaaaccaacgtcttgtctccgcatacaagtgggtccatcagccgccggaggacacccatgaagctaatgaagacggtgatgactgtcttaggggcgtttgtggtatgctggaccccgggcctggtggttctgctcctcgacggcctgaactgcaggcagtgtggcgtgcagcatgtgaaaaggtggttcctgctgctggcgctgctcaactccgtcgtgaaccccatcatctactcctacaaggacgaggacatgtatggcaccatgaagaagatgatctgctgcttctctcaggagaacccagagaggcgtccctctcgcatcccctccacagtcctcagcaggagtgacacaggcagccagtacatagaggatagtattagccaaggtgcagtctgcaataaaagcacttcc

SEQ ID NO: 52 - protein, 398 aa, human
MESGLLRPAPVSEVIVLHYNYTGKLRGARYQPGAGLRADAVVCLAVCAFIVLENLAVLLVLGRHPRFHAPMFLLLGSLTLSDLLAGAAYAANILLSGPLTLKLSPALWFAREGGVFVALTASVLSLLAIALERSLTMARRGPAPVSSRGRTLAMAAAAWGVSLLLGLLPALGWNCLGRLDACSTVLPLYAKAYVLFCVLAFVGILAAICALYARIYCQVRANARRLPARPGTAGTTSTRARRKPRSLALLRTLSVVLLAFVACWGPLFLLLLLDVACPARTCPVLLQADPFLGLAMANSLLNPIIYTLTNRDLRHALLRLVCCGRHSCGRDPSGSQQSASAAEASGGLRRCLPPGLDGSFSGSERSSPQRDGLDTSGSTGSPGAPTAARTLVSEPAAD

SEQ ID NO: 53 - DNA, 1194 bp, human
atggagtcggggctgctgcggccggcgccggtgagcgaggtcatcgtcctgcattacaactacaccggcaagctccgcggtgcgcgctaccagccgggtgccggcctgcgcgccgacgccgtggtgtgcctggcggtgtgcgccttcatcgtgctagagaatctagccgtgttgttggtgctcggacgccacccgcgcttccacgctcccatgttcctgctcctgggcagcctcacgttgtcggatctgctggcaggcgccgcctacgccgccaacatcctactgtcggggccgctcacgctgaaactgtcccccgcgctctggttcgcacgggagggaggcgtcttcgtggcactcactgcgtccgtgctgagcctcctggccatcgcgctggagcgcagcctcaccatggcgcgcagggggcccgcgcccgtctccagtcgggggcgcacgctggcgatggcagccgcggcctggggcgtgtcgctgctcctcgggctcctgccagcgctgggctggaattgcctgggtcgcctggacgcttgctccactgtcttgccgctctacgccaaggcctacgtgctcttctgcgtgctcgccttcgtgggcatcctggccgcgatctgtgcactctacgcgcgcatctactgccaggtacgcgccaacgcgcggcgcctgccggcacggcccgggactgcggggaccacctcgacccgggcgcgtcgcaagccgcgctcgctggccttgctgcgcacgctcagcgtggtgctcctggcctttgtggcatgttggggccccctcttcctgctgctgttgctcgacgtggcgtgcccggcgcgcacctgtcctgtactcctgcaggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcagac

SEQ ID NO: 54 - DNA, 22 bp, Artificial Sequence
  Primer
ccaccgacccatgtactatttt

SEQ ID NO: 55 - DNA, 22 bp, Artificial Sequence
  Primer
tgtaggctactcctgccaacag

SEQ ID NO: 56 - DNA, 22 bp, Artificial Sequence
  Probe, labeled 5′-terminal with FAM and 3′-terminal with TAMRA
ttggcaatctggccctctcaga

SEQ ID NO: 57 - DNA, 21 bp, Artificial Sequence
  Primer
actgtcagcacatggctcctt

SEQ ID NO: 58 - DNA, 21 bp, Artificial Sequence
  Primer
accgtaatgtgcctctcgatt

SEQ ID NO: 59 - DNA, 22 bp, Artificial Sequence
  Probe, labeled 5′-terminal with FAM and 3′-terminal with TAMRA
attgacaccagcctgacggcat

SEQ ID NO: 60 - DNA, 19 bp, Artificial Sequence
  Primer
ccgtgctcttcttggtcat

SEQ ID NO: 61 - DNA, 19 bp, Artificial Sequence
  Primer
ccagatggcaatcaaaacc

SEQ ID NO: 62 - DNA, 26 bp, Artificial Sequence
  Probe, labeled 5′-terminal with FAM and 3′-terminal with TAMRA
tgcagcttcatcgtcttggagaacct

SEQ ID NO: 63 - DNA, 22 bp, Artificial Sequence
  Primer
cctggtcaagactgttgtcatc

SEQ ID NO: 64 - DNA, 19 bp, Artificial Sequence
  Primer
caggacattgcaggactca

SEQ ID NO: 65 - DNA, 27 bp, Artificial Sequence
  Probe, labeled 5′-terminal with FAM and 3′-terminal with TAMRA
tggtactgctcctggatggtttaggct

SEQ ID NO: 66 - DNA, 19 bp, Artificial Sequence
  Primer
ccaacaaggtccaggaaca

SEQ ID NO: 67 - DNA, 19 bp, Artificial Sequence
  Primer
aggttttccaccacaatgg

SEQ ID NO: 68 - DNA, 28 bp, Artificial Sequence
  Probe, labeled 5′-terminal with FAM and 3′-terminal with TAMRA
aattataccaaggagacgctggaaacgc

SEQ ID NO: 69 - DNA, 19 bp, Artificial Sequence
  Primer
gaactgcctgtgcgccttt

SEQ ID NO: 70 - DNA, 20 bp, Artificial Sequence
  Primer
ccatagaggcccatgatggt

SEQ ID NO: 71 - DNA, 28 bp, Artificial Sequence
  Probe, labeled 5′-terminal with FAM and 3′-terminal with TAMRA
tctgcccctctactccaagcgctacatc

SEQ ID NO: 72 - DNA, 19 bp, Artificial Sequence
  Primer
tgactgcttccctcaccaa

SEQ ID NO: 73 - DNA, 22 bp, Artificial Sequence
  Primer
gcatcctcatgattgacatgtg

SEQ ID NO: 74 - DNA, 22 bp, Artificial Sequence
  Probe, labeled 5′-terminal with FAM and 3′-terminal with TAMRA
ttgctggttatcgccgtggaga

SEQ ID NO: 75 - DNA, 19 bp, Artificial Sequence
  Primer
cttgctccactgtcttgcc

SEQ ID NO: 76 - DNA, 19 bp, Artificial Sequence
  Primer
tagagtgcacagatcgcgg

SEQ ID NO: 77 - DNA, 28 bp, Artificial Sequence
  Probe, labeled 5′-terminal with FAM and 3′-terminal with TAMRA
ctctacgccaaggcctacgtgctcttct

(19) United States
(12) Patent Application Publication
(10) Pub. No.: US 2005/0153289 A1
(43) Pub. Date: Jul. 14, 2005

(54) Method of analyzing gene expression

(76) Inventors: Shuji Hinuma, Ibaraki (JP); Makoto Kobayashi, Hyogo (JP); Toshimitsu Arai, Osaka (JP); Shoji Fukusumi, Ibaraki (JP); Ryo Fujii, Ibaraki (JP); Hidetoshi Komatsu, Ibaraki (JP); Fumika Matsumura, Ibaraki (JP); Yuji Kawamata, Ibaraki (JP); Kazuhiro Ogi, Ibaraki (JP)

Correspondence Address: EDWARDS & ANGELL, LLP, P.O. BOX 55874, BOSTON, MA, 02205, US

(21) Appl. No.: 10/498,848

(22) PCT Filed: Dec. 13, 2002
(86) PCT No.: PCT/JP02/13097

(30) Foreign Application Priority Data
Dec. 14, 2001 (JP) 2001-382053
Feb. 21, 2002 (JP) 2002-45104
May 15, 2002 (JP) 2002-140111
Nov. 18, 2002 (JP) 2002-333769

(51) Int. Cl. C12Q 1/68; G01N 33/574; C07K 16/30; C07H 21/04; C07K 14/42

(52) U.S. Cl. 435/6; 435/7.23; 530/388.8; 530/396; 536/24.3

(57) ABSTRACT

The present invention intends to provide a method for analyzing gene expression, a novel use for a receptor protein and the like. Specifically, the present invention provides a method for analyzing gene expression, which is characterized by identifying genes, which are characteristically promoted or suppressed in certain cells or tissues by quantitatively analyzing the individual expressed amounts of a plural number of genes collectively, an assay kit employed in the method and the like. The present invention also provides a method of screening compounds or salts thereof which alter the binding properties of a novel G protein coupled receptor protein comprising an amino acid sequence identical or substantially identical to the amino acid sequence represented by SEQ ID NO: 9 or a salt thereof with a ligand, characterized by the use of the protein or salt thereof and a protein (such as a lectin) showing an affinity for a sugar chain which is one of the ligands.

### TECHNICAL FIELD

The present invention relates to a method of analyzing gene expression wherein the expression of multiple genes is analyzed collectively, to a gene expression analysis kit used therefor and the like. The present invention also relates to a method of specifying disease-associated genes employing said gene expression analysis method and analysis kit. Furthermore, the present invention relates to a method of diagnosing specific diseases by analyzing expressed amounts and mutations of disease-associated genes, and to drugs comprising specified gene DNA or gene products thereof. In addition, the present invention relates to uses for a human-derived G protein-coupled receptor protein (dJ287G14.2 receptor) and a polynucleotide encoding therefor. Moreover, the present invention relates to a novel mouse-derived G protein-coupled receptor protein (dJ287G14.2 receptor), to a polynucleotide encoding therefor and to uses for these. In addition, the present invention relates to a method for screening EDG-1 receptor agonists and antagonists or EDG-2 agonists and antagonists.

### BACKGROUND ART

G protein-coupled receptor proteins are present on the various functional cell surfaces of cells and organs in vivo, and play an important physiological role as targets for molecules such as hormones, neurotransmitters, bioactive substances and the like which regulate the functions of these cells and organs. When it binds with a bioactive substance a receptor transmits a signal inside the cell, and this signal stimulates a variety of reactions such as cell activation or suppression. Elucidation of substances which regulate complex functions within various cells and organs in vivo and their relationships with specific receptor proteins and G protein-coupled receptor proteins offers an extremely useful tool for developing drugs closely associated with the functions of various cells and organs in vivo: about 45% of existing drugs act on receptor proteins on cell membranes, and of these the G protein-coupled receptor proteins are known to play an important role (Drews, J., Science 287, 1960 (2000)).

Ion channels are membrane proteins, which penetrate the cell membrane and play a role in cell response by regulating membrane permeation of ions. Because they are ion-selective they are known as Na⁺, K⁺, Ca²⁺ and Cl⁻ channels and the like, and are classified as either potential-dependent (Na⁺, K⁺, Ca²⁺) or receptor-dependent (nicotinic cholinergic receptor, GABA receptor) depending on the channel opening and closing mechanism. These ion channels have many physiological functions in vivo. For example, the K⁺ channel is a membrane protein which selectively allows permeation of K⁺ ions, and in vivo it is closely associated with important physiological functions including cell resting membrane potential formation, repolarization, and regulating frequency of action potential occurrence. The Cl⁻channel exhibits a variety of physiological functions including regulation of neuromuscular excitability, cell volume regulation, and electrolyte and water transport in the epithelial cell membranes. Thus, the various ion channels are drug targets because they have a variety of physiological functions.

Some receptors in the body also have enzyme activity, such as the tyrosine kinase receptors for example. Because this enzyme activity serves a variety of physiological functions, tyrosine kinase receptors have become the target of many drugs. Other gene families include transcription factors, transporters, protein kinases, protein phosphatases, proteases, heat shock proteins, ATPases, DNA-binding proteins and the like, which constitute families of tens or hundreds and are drug targets.

A variety of genes (about 400 G protein-coupled receptors, about 100 tyrosine kinase receptors and about 150 ion channels) have already been identified and cloned. However, the functions of all of these genes have not been elucidated, and there are many proteins whose functions are unknown. Elucidating the functions of these proteins is the first step in developing novel drugs.

Methods, which have been used for analyzing genes expressed in vivo, include the Northern blot method, differential display method and RT-PCR method. However, there are limits on the number of genes that can be analyzed at once, and it has been difficult to process samples containing many genes. Sensitivity has also been low for quantification.

A method that has been developed in recent years for analyzing all genes expressed in vivo is the microarray method, in which thousands or tens of thousands of pieces of DNA data are synthesized or spotted on a glass slide or other chip, target DNA derived from the RNA under analysis is hybridized, and the amount of transcripted gene is measured using the resulting hybrid as the indicator, but sensitivity has not been particularly high for quantification.

Methods of assaying specific mRNA include the TaqMan method and the like, and for example a method of assaying hTERT mRNA is disclosed in Kokai No. 2001-204483. The TaqMan method is also known as one method of SNP analysis, in which primers and probes, which recognize the SNP, are designed and can be used to analyze not only expressed amounts but also disease-related mutations.

A protein having the identical amino acid sequence as the dJ287G14.2 receptor used in the present invention and DNA encoding therefor have been described (WO 2001/18207). However, the functions of these G protein-coupled receptor proteins and their physiological ligands have not been elucidated.

An EDG-1 receptor (Biochem Biophys Res Commun Nov. 26, 1997; 240(3): 737-41), EDG-2 receptor (Biochem Biophys Res Commun Feb. 24, 1997; 231(3): 619-22), EDG-3 receptor (Cell Oct. 29, 1999; 99(3): 301-12), EDG-5 receptor (J Biol Chem Dec. 10, 1999; 274(50): 35343-50) and EDG-8 receptor (Biochemistry Nov. 20, 2001; 40(46): 14053-60) have been described, along with the fact that these receptors are expressed in vascular tissue. However, it has not been explained to what degree each receptor is expressed in vascular and other cells. Clear expression of EDG-4, EDG-6 and EDG-7 in vascular tissue is unknown.

Notwithstanding, until now there has been no idea for assaying the expression of many genes belonging to a specific family all at once with high sensitivity, or any assay system for realizing such an idea. Consequently if there were a system for assaying the expression of many genes belonging to a specific family all at once with high sensitivity, it would be useful in such areas as specifying disease genes and diagnosing and treating specific diseases, since genes which are highly expressed specifically in certain disease cells or the like could be identified relatively and quantitatively.

### DISCLOSURE OF THE INVENTION

Namely, as shown below, the present invention provides a method for analyzing gene expression, a method of diagnosis, an assay kit used in these methods, and proteins and uses for proteins whose functions are elucidated by such methods or kit.

[1] A method for analyzing gene expression, wherein genes whose expression is characteristically promoted or inhibited in certain cells or tissue are identified by quantitatively analyzing the individual expressed amounts of multiple genes collectively.

[2] The method according to [1], wherein expression analysis is performed collectively on multiple genes belonging to a specific gene family in order to identify the genes in that family whose expression is characteristically promoted or inhibited in certain cells or tissue by computing expressed amounts as absolute values.

[3] The method according to [1], wherein an amplification reaction is performed by bringing an mRNA sample which may contain multiple mRNA targets into contact at multiple reaction sites with individual amplification reagents each of which comprises a primer pair corresponding to a particular mRNA target, and gene expression analysis is performed by measuring the amounts produced in the resulting amplification product.

[4] The method according to [1], wherein an amplification reaction is performed by bringing an mRNA sample which may contain multiple mRNA targets into contact, at the respective reaction sites of a reaction device having multiple reaction sites, with individual amplification reagents each of which comprises a primer pair corresponding to a particular mRNA target, and gene expression analysis is performed by measuring the amounts produced in the resulting amplification product.

[5] The method according to [2], wherein the specific gene family is the G protein-coupled receptor gene family.

[6] The method according to [2], wherein the specific gene family is the tyrosine kinase receptor gene family.

[7] The method according to [2], wherein the specific gene family is the ion channel gene family.

[8] The method according to [2], wherein the specific gene family is a gene family associated with either transcription factors, transporters, protein kinases, protein phosphatases, proteases, heat shock proteins, ATPases or DNA-binding proteins.

[9] A drug comprising a gene or product of a gene, which is specified by a method described in any of [1] to [8], and the expression of which is characteristically promoted or inhibited in certain cells or tissue.

[10] The method according to [3] or [4], wherein the reaction device is a plate having multiple wells as reaction sites.

[11] The method according to [10], wherein the plate is a 96-well or 384-well plate.

[12] The method according to [3] or [4], wherein 10-800 primer pairs are used.

[13] The method according to [3] or [4], wherein 10-300 primer pairs are used.

[14] The method according to [3], [4], [12] or [13], wherein the amplification reaction is a polymerase chain reaction.

[15] The method according to [14], wherein SNP analysis is performed.

[16] The method according to [3] or [4], wherein the produced amounts of amplification products are measured using probes, which are complementary or substantially complementary to said amplification products.

[17] The method according to [16], wherein the probes are probes which hybridize with mRNA.

[18] The method according to 117], wherein the probes are fluorescence labeled probes.

[19] The method according to [3] or [4], wherein a normal human-derived mRNA sample and an mRNA sample derived from a patient with a specific disease are used as the mRNA samples.

[20) The method according to [19], wherein mRNA whose expression is promoted or inhibited in an mRNA sample derived from a disease patient is specified, and a gene encoding said mRNA is designated as a disease-associated gene for that disease.

[21] The method according to [20], wherein the specific gene family is the G protein-coupled receptor protein gene family, and wherein a cancer-related gene is specified by using mRNA derived from a cancer patient.

[22] A primer pair kit comprising two or more pairs of primers each consisting of a first primer which is complementary or substantially complementary to one chain of an exon region of a target gene sequence and a second primer which is complementary or substantially complementary to the other chain of the exon region of the target gene sequence.

[23] The primer pair kit according to [22], wherein the target gene is a human G protein-coupled receptor protein gene, tyrosine kinase receptor gene or ion channel gene.

[24] The primer pair kit according to [23], composed of 10-800 primer pairs.

[25] The primer pair kit according to [23], composed of 10-300 primer pairs.

[26] The primer pair kit according to [23], for purposes of specifying disease-associated genes.

[27] An mRNA assay kit having each reaction site in a reaction device with multiple reaction sites filled with an individual amplification reagent comprising a primer pair corresponding to a particular mRNA target.

[28] The kit according to [27], also comprising a fluorescent probe.

[29] The kit according to [28], also comprising Tth DNA polymerase.

[30] A method of diagnosing a patient's disease using the method according to any of [1] to [4] or the assay kit according to [27] by assaying the mRNA of multiple target disease genes which may be contained in an mRNA sample collected from the patient, or by measuring-the mutated amount of said mRNA.

[31] The diagnostic method according to [30], wherein cancer is diagnosed by identifying cancer-associated human G protein-coupled receptor protein genes.

[32] A drug comprising an agonist, antagonist or antibodies to the gene product of a gene identified by the diagnostic method according to [30], or DNA encoding said gene product.

[33] The drug according to [32], which is a cancer therapy drug.

[34] A method of administering an agonist, antagonist or antibodies to the gene product of a gene identified by the diagnostic method according to [30] or DNA encoding said gene product in order to treat diseases involving that gene.

[35] The treatment method according to [34], wherein the disease is cancer.

It was discovered using the method for analyzing gene expression of the present invention that the dJ287G14.2 receptor is highly expressed in prostate cancer cells. Based on this finding, the present invention provides a novel use for a receptor protein or partial peptide or salt thereof (dJ287G14.2 receptor), for polynucleotides encoding that receptor protein or partial peptide thereof (DNA, RNA and derivatives thereof) and for antibodies to that receptor protein or partial peptide or salt thereof and the like. A novel mouse-derived dJ287G14.2 receptor, polynucleotides (DNA, RNA and derivatives thereof) encoding that receptor protein or partial peptide thereof, and antibodies to that receptor protein or partial peptide or salt thereof and the like are also provided.

Namely,

[36] A drug containing a receptor protein which contains an amino acid sequence identical to or substantially identical to the amino acid sequence represented by SEQ ID NO: 9, or a partial peptide or salt thereof;

[37] The drug according to [37], which is a birth inducer or a preventative and/or therapeutic drug for cancer, prostatomegaly, male gonad dysfunction, infertility, premature birth, endometriosis, cirrhosis of the liver, hepatitis, hepatic insufficiency or calculus;

[38] The drug according to [37], wherein the cancer is prostate cancer, non-small cell carcinoma, ovarian cancer, stomach cancer, bladder cancer, breast cancer, cervical cancer, colon cancer, rectal cancer or large intestinal cancer;

[39] A diagnostic drug containing antibodies to a protein which contains an amino acid sequence identical to or substantially identical to the amino acid sequence represented by SEQ ID NO: 9, or to a partial peptide or salt thereof;

[40] The diagnostic drug according to [40], which is a diagnostic drug for cancer, prostatomegaly, male gonad dysfunction, infertility, premature birth, endometriosis, cirrhosis of the liver, hepatitis, hepatic insufficiency or calculus;

[41] A drug containing antibodies to a protein which contains an amino acid sequence identical to or substantially identical to the amino acid sequence represented by SEQ ID NO: 9, or to a partial peptide or salt thereof;

[42] The drug according to [41], which is a birth inducer or a preventative and/or therapeutic drug for cancer, prostatomegaly, male gonad dysfunction, infertility, premature birth, endometriosis, cirrhosis of the liver, hepatitis, hepatic insufficiency or calculus;

[43] A method of screening compounds or salts of compounds which alter the binding properties of a G protein-coupled receptor protein containing an amino acid sequence identical to or substantially identical to the amino acid sequence represented by SEQ ID NO: 9 or of a salt thereof with a protein showing affinity for a sugar chain, characterized by the use of (1) the aforementioned G protein-coupled receptor protein, or a partial peptide or salt thereof, and (2) the protein showing affinity for a sugar chain;

[44] The screening method according to [43], wherein the protein showing affinity for a sugar chain is a protein showing affinity for an asparagine-linked sugar chain or a serine/threonine-linked sugar chain;

[45] The screening method according to [43], wherein the protein showing affinity for a sugar chain is a lectin;

[46] The screening method according to [43], wherein the protein showing affinity for a sugar chain is concanavalin A, lentil lectin, pea lectin, datura lectin, Maackia Amurensis lectin or phytohemagglutinin;

[47] A screening kit for compounds or salts of compounds which alter the binding properties of a G protein-coupled receptor protein containing an amino acid sequence identical to or substantially identical to the amino acid sequence represented by SEQ ID NO:9 or of a salt thereof with a protein showing affinity for a sugar chain, characterized in that it contains (1) the aforementioned G protein-coupled receptor protein, or a partial peptide or salt thereof, and (2) the protein showing affinity for a sugar chain;

[48] A compound or salt of a compound which alters the binding properties of a protein showing affinity for a sugar chain with a G protein-coupled receptor protein containing an amino acid sequence identical to or substantially identical to the amino acid sequence represented by SEQ ID NO: 9 or with a salt thereof, obtained using the screening method according to [46] or the screening kit according to [47];

[49] A drug containing a compound or salt thereof according to [48];

[50] The drug according to [49], which is a birth inducer or a preventative and/or therapeutic drug for cancer, prostatomegaly, male gonad dysfunction, infertility, premature birth, endometriosis, cirrhosis of the liver, hepatitis, hepatic insufficiency or calculus;

[51] The drug according to [50], wherein the cancer is prostate cancer, non-small cell carcinoma, ovarian cancer, stomach cancer, bladder cancer, breast cancer, cervical cancer, colon cancer, rectal cancer or large intestinal cancer;

[52] A method of screening birth inducers or compounds or salts of compounds for preventing and/or treating cancer, prostatomegaly, male gonad dysfunction, infertility, premature birth, endometriosis, cirrhosis of the liver, hepatitis, hepatic insufficiency or calculus which alter the expressed amount of a G protein-coupled receptor protein containing an amino acid sequence identical to or substantially identical to the amino acid sequence represented by SEQ ID NO: 9, characterized by the use of a polynucleotide containing a polynucleotide encoding the aforementioned G protein-coupled receptor protein or a partial peptide thereof;

[53] A screening kit birth inducers or compounds or salts of compounds for preventing and/or treating cancer, prostatomegaly, male gonad dysfunction, infertility, premature birth, endometriosis, cirrhosis of the liver, hepatitis, hepatic insufficiency or calculus which alter the expressed amount of a G protein-coupled receptor protein containing an amino acid sequence identical to or substantially identical to the amino acid sequence represented by SEQ ID NO: 9, wherein is contained a polynucleotide containing a polynucleotide encoding the aforementioned G protein-coupled receptor protein or a partial peptide thereof;

[54] A birth inducer or compound or salt of a compound for preventing and/or treating cancer, prostatomegaly, male gonad dysfunction, infertility, premature birth, endometriosis, cirrhosis of the liver, hepatitis, hepatic insufficiency or calculus which alters the expressed amount of a G protein-coupled receptor protein containing an amino acid sequence identical to or substantially identical to the amino acid sequence represented by SEQ ID NO: 9 or of a partial peptide thereof, obtained using the screening method according to [52] or the screening kit according to [53];

[55] A-birth inducer or preventative and/or therapeutic agent for cancer, prostatomegaly, male gonad dysfunction, infertility, premature birth, endometriosis, cirrhosis of the liver, hepatitis, hepatic insufficiency or calculus, containing a compound or salt thereof according to [54];

[56] The agent according to [55], wherein the cancer is prostate cancer, non-small cell carcinoma, ovarian cancer, stomach cancer, bladder cancer, breast cancer, cervical cancer, colon cancer, rectal cancer or large intestinal cancer;

[57] A signal transmission enhancing agent for a G protein-coupled receptor protein containing an amino acid sequence identical to or substantially identical to the amino acid sequence represented SEQ ID NO: 9, containing a protein showing affinity for a sugar chain;

[58] The agent according to [57], which is a birth inducer or preventative and/or therapeutic agent for cancer, prostatomegaly, male gonad dysfunction, infertility, premature birth, endometriosis, cirrhosis of the liver, hepatitis, hepatic insufficiency or calculus;

[59] The agent according to [58], wherein the cancer is prostate cancer, non-small cell carcinoma, ovarian cancer, stomach cancer, bladder cancer, breast cancer, cervical cancer, colon cancer, rectal cancer or large intestinal cancer;

[60] A birth inducer or preventative and/or therapeutic agent for cancer, prostatomegaly, male gonad dysfunction, infertility, premature birth, endometriosis, cirrhosis of the liver, hepatitis, hepatic insufficiency or calculus, containing a polynucleotide which contains a polynucleotide encoding a G protein-coupled receptor protein containing an amino acid sequence identical to or substantially identical to the amino acid sequence represented by SEQ ID NO: 9, or a partial peptide thereof;

[61] The agent according to [60], wherein the cancer is prostate cancer, non-small cell carcinoma, ovarian cancer, stomach cancer, bladder cancer, breast cancer, cervical cancer, colon cancer, rectal cancer or large intestinal cancer;

[62] A diagnostic drug for cancer, prostatomegaly, male gonad dysfunction, infertility, premature birth, endometriosis, cirrhosis of the liver, hepatitis, hepatic insufficiency or calculus, containing a polynucleotide which contains a polynucleotide encoding a G protein-coupled receptor protein containing an amino acid sequence identical to or substantially identical to the amino acid sequence represented by SEQ ID NO: 9, or a partial peptide thereof;

[63] The diagnostic drug according to [62], wherein the cancer is prostate cancer, non-small cell carcinoma, ovarian cancer, stomach cancer, bladder cancer, breast cancer, cervical cancer, colon cancer, rectal cancer or large intestinal cancer;

[64] A birth inducer or preventative and/or therapeutic agent for cancer, prostatomegaly, male gonad dysfunction, infertility, premature birth, endometriosis, cirrhosis of the liver, hepatitis, hepatic insufficiency or calculus, containing an antisense polynucleotide which contains a nucleotide sequence or part of a nucleotide sequence complementary to a polynucleotide containing a polynucleotide which encodes a G protein-coupled receptor protein containing an amino acid sequence identical to or substantially identical to the amino acid sequence represented by SEQ ID NO: 9, or a partial peptide thereof;

[65] The agent according to [64], wherein the cancer is prostate cancer, non-small cell carcinoma, ovarian cancer, stomach cancer, bladder cancer, breast cancer, cervical cancer, colon cancer, rectal cancer or large intestinal cancer;

[66] A diagnostic drug for cancer, prostatomegaly, male gonad dysfunction, infertility, premature birth, endometriosis, cirrhosis of the liver, hepatitis, hepatic insufficiency or calculus, containing an antisense polynucleotide which contains a nucleotide sequence or part of a nucleotide sequence complementary to a polynucleotide containing a polynucleotide which encodes a G protein-coupled receptor protein containing an amino acid sequence identical to or substantially identical to the amino acid sequence of SEQ ID NO: 9, or a partial peptide thereof;

[67) The diagnostic drug according to [66], which is a diagnostic drug for cancer;

[68] The diagnostic drug according to [67], wherein the cancer is prostate cancer, non-small cell carcinoma, ovarian cancer, stomach cancer, bladder cancer, breast cancer, cervical cancer, colon cancer, rectal cancer or large intestinal cancer;

[69] A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized in that an effective dose of a receptor protein containing an amino acid sequence identical to or substantially identical to the amino acid sequence represented by SEQ ID NO: 9, or a partial peptide or salt thereof, is administered to mammals;

[70] A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized in that an effective dose of antibodies to a protein containing an amino acid sequence identical to or substantially identical to the amino acid sequence represented by SEQ ID NO: 9, or to a partial peptide or salt thereof, is administered to mammals;

[71] A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized in that an effective dose of a compound or salt thereof which alters the binding properties of a G protein-coupled receptor protein containing an amino acid sequence identical to or substantially identical to the amino acid sequence represented by SEQ ID NO: 9, or of a partial peptide or salt thereof, with a protein showing affinity for a sugar chains, is administered to mammals;

[72] A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized in that an effective dose of a compound or salt thereof which alters the expressed amount of a G protein-coupled receptor protein containing an amino acid sequence identical to or substantially identical to the amino acid sequence represented by SEQ ID NO: 9 is administered to mammals;

[73] A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized in that an effective dose of a protein or salt thereof showing affinity for a sugar chain is administered to mammals;

[74] A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized in that an effective dose of an antisense polynucleotide containing a nucleotide sequence or part of a nucleotide sequence complementary to a polynucleotide encoding a protein or partial peptide of a protein containing an amino acid sequence identical to or substantially identical to the amino acid sequence represented by SEQ ID NO: 9 is administered to mammals;

[75] A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized in that an effective dose of a polynucleotide containing a polynucleotide which encodes a G protein-coupled receptor protein containing an amino acid sequence identical to or substantially identical to the amino acid sequence represented by SEQ ID NO: 9, or a partial peptide thereof, is administered to mammals;

[76] The use of a receptor protein containing an amino acid sequence identical to or substantially identical to the amino acid sequence represented by SEQ ID NO: 9, or a partial peptide or salt thereof, to manufacture a birth inducer or a preventative and/or therapeutic agent for cancer, prostatomegaly, male gonad dysfunction, infertility, premature birth, endometriosis, cirrhosis of the liver, hepatitis, hepatic insufficiency or calculus;

[77] The use of antibodies to a receptor protein containing an amino acid sequence identical to or substantially identical to the amino acid sequence represented by SEQ ID NO: 9, or to a partial peptide or salt thereof, to manufacture a birth inducer or a preventative and/or therapeutic agent for cancer, prostatomegaly, male gonad dysfunction, infertility, premature birth, endometriosis, cirrhosis of the liver, hepatitis, hepatic insufficiency or calculus;

[78] The use of a compound or salt thereof which alters the binding properties of a protein showing affinity for a sugar chain with a G protein-coupled receptor protein containing an amino acid sequence identical to or substantially identical to the amino acid sequence represented by SEQ ID NO: 9, or with a partial peptide or salt thereof, to manufacture a birth inducer or a preventative and/or therapeutic agent for cancer, prostatomegaly, male gonad dysfunction, infertility, premature birth, endometriosis, cirrhosis of the liver, hepatitis, hepatic insufficiency or calculus;

[79] The use of a compound or salt thereof which alters the expressed amount of a G protein-coupled receptor protein containing an amino acid sequence identical to or substantially identical to the amino acid sequence represented by SEQ ID NO: 9 to manufacture a birth inducer or a preventative and/or therapeutic agent for cancer, prostatomegaly, male gonad dysfunction, infertility, premature birth, endometriosis, cirrhosis of the liver, hepatitis, hepatic insufficiency or calculus;

[80] The use of a protein or salt thereof showing affinity for a sugar chain to manufacture a birth inducer or a preventative and/or therapeutic agent for cancer, prostatomegaly, male gonad dysfunction, infertility, premature birth, endometriosis, cirrhosis of the liver, hepatitis, hepatic insufficiency or calculus;

[81] The use of an antisense polynucleotide containing a nucleotide sequence or part of a nucleotide sequence complementary to a polynucleotide which encodes a protein containing an amino acid sequence identical to or substantially identical to the amino acid sequence represented by SEQ ID NO: 9 or a partial peptide thereof to manufacture a birth inducer or a preventative and/or therapeutic agent for cancer, prostatomegaly, male gonad dysfunction, infertility, premature birth, endometriosis, cirrhosis of the liver, hepatitis, hepatic insufficiency or calculus;

[82] The use of a polynucleotide containing a polynucleotide encoding a G protein-coupled receptor protein containing an amino acid sequence identical to or substantially identical to the amino acid sequence represented by SEQ ID NO: 9 or a partial peptide thereof to manufacture a birth inducer or a preventative and/or therapeutic agent for cancer, prostatomegaly, male gonad dysfunction, infertility, premature birth, endometriosis, cirrhosis of the liver, hepatitis, hepatic insufficiency or calculus;

[83] A G protein-coupled receptor protein containing an amino acid sequence identical to or substantially identical to the amino acid sequence represented by SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 27, or a salt thereof;

[84] A G protein-coupled receptor protein consisting of an amino acid sequence represented by SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 27, or a salt thereof;

[85] A polynucleotide containing the polynucleotide encoding the G protein-coupled receptor protein according to [81];

[86] DNA consisting of a nucleotide sequence represented by SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28;

[87] A recombinant vector containing the polynucleotide according to [83];

[88] A transformant transformed by the recombinant vector according to [87];

[89] A method of manufacturing the G protein-coupled receptor protein or salt thereof according to [83], wherein the transformant according to [88] is cultured in order to produce the G protein-coupled receptor protein or salt thereof according to [83];

[90] Antibodies to the G protein-coupled receptor protein or salt thereof according to [83];

[91] DNA, which hybridizes under highly stringent conditions with DNA, consisting of a nucleotide sequence represented by SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28;

[92] A polynucleotide containing a nucleotide sequence or part of a nucleotide sequence complementary to the polynucleotide according to [85].

It was also discovered using the method for analyzing gene expression of the present invention that EDG-1 receptors are highly expressed in vascular endothelial cells and EDG-2 receptors in vascular smooth muscle cells. Based on this finding, the present invention provides a method of screening EDG-1 receptor agonists and antagonists using vascular endothelial cells or vascular smooth muscle cells, or for EDG-2 receptor agonists or antagonists using vascular smooth muscle cells.

[93] A method for screening EDG-1 receptor agonists or antagonists, characterized by the use of vascular endothelial cells.

[94] The screening method according to [93], wherein the EDG-1 receptor is a G protein-coupled receptor protein containing an amino acid sequence identical to or substantially identical to the amino acid sequence represented by SEQ ID NO: 38, or a partial peptide or salt thereof.

[95] A screening kit for EDG-1 receptor agonists or antagonists, wherein are contained vascular endothelial cells.

[96] An EDG-1 receptor agonist or antagonist obtained using a screening method according to [93] or a screening kit according to [95].

[97] A preventative and/or therapeutic agent for arteriosclerosis, myocardial infarction, cerebral infarction or ischemic disease, containing an EDG-1 receptor agonist obtained using the screening method according to 93 above or the screening kit according to [85].

[98] A method of screening EDG-2 receptor agonists or antagonists, characterized by the use of vascular smooth muscle cells.

[99] The screening method according to [98], wherein the EDG-2 receptor is a G protein-coupled receptor protein containing an amino acid sequence identical to or substantially identical to the amino acid sequence represented by SEQ ID NO: 40, or a partial peptide or salt thereof.

[100] A kit for screening EDG-2 receptor agonists or antagonists, wherein are contained vascular smooth muscle cells.

[101] An EDG-2 receptor agonist or antagonist obtained using the screening method according to [98] or the screening kit according to [100].

[102] A preventative and/or therapeutic agent for arteriosclerosis, myocardial infarction, cerebral infarction or ischemic disease, containing an EDG-2 receptor agonist or antagonist obtained using the screening method according to [98] or the screening kit according to [100].

[103] A method of preventing and/or treating arteriosclerosis, myocardial infarction, cerebral infarction or ischemic disease, characterized in that an effective dose of an EDG-1 receptor agonist obtained using the screening method according to [93] or the screening kit according to [95], or an EDG-2 receptor antagonist obtained using the screening method according to [98] or the screening kit according to [100], is administered to mammals.

[104] The use of an EDG-1 receptor agonist obtained using the screening method according to [93] or the screening kit according to [95] or an EDG-2 receptor antagonist obtained using the screening method according to [98] or the screening kit according to [100] to manufacture a preventative and/or therapeutic agent for arteriosclerosis, myocardial infarction, cerebral infarction or ischemic disease, are provided by the present invention.

### BRIEF DESCRIPTION OF THE DRAWINGS

FIG. 1 shows dJ287G14.2 expression in various cell lines.

FIG. 2 shows the distribution of dJ287G14.2 receptor mRNA expression in various human tissues.

FIG. 3 shows the amino acid sequence of the dJ287G14.2 receptor.

FIG. 4 is a continuation of FIG. 3, showing the amino acid sequence of the dJ287G14.2 receptor.

FIG. 5 shows the nucleotide sequence of DNA encoding the dJ287G14.2 receptor.

FIG. 6 is a continuation of FIG. 5, showing the nucleotide sequence of DNA encoding the dJ287G 14.2 receptor.

FIG. 7 is a continuation of FIG. 6, showing the nucleotide sequence of DNA encoding the dJ287G14.2 receptor.

FIG. 8 is a continuation of FIG. 7, showing the nucleotide sequence of DNA encoding the dJ287G14.2 receptor.

FIG. 9 shows the results of an investigation into the effects of lectins on the amount of cAMP produced in CHO cells expressing a dJ287G14.2-GFP fused protein, with the horizontal axis indicating lectin concentration and the vertical axis cAMP production, where ConA is concanavalin A, Lentil is lentil lectin, Pea is pea lectin, and FSK is forskolin (Mean±s.d. (n=2)).

FIG. 10 shows the results of an investigation into the effects of lectins on the amount of cAMP produced in mock CHO cells, with the horizontal axis indicating lectin concentration and the vertical axis cAMP production (mean values for n=2), where ConA is concanavalin A, Lentil is lentil lectin, Pea is pea lectin, and FSK is forskolin (mean i s.d. (n=2)).

FIG. 11 shows the results of observation with a confocal microscope (Leica) of the fluorescent image of GFP in a dJ287G14.2-GFP fused protein expressed in CHO cells.

FIG. 12 shows the results of observation with a confocal microscope (Leica) of the fluorescent image of GFP when concanavalin A was added to dJ287G14.2-GFP fused protein expressed in CHO cells.

### BEST MODE FOR CARRYING OUT THE INVENTION

The present invention is described in detail below.

The present invention is characterized in that a gene the expression of which is characteristically promoted or inhibited in specific cells or tissue is identified by quantitatively analyzing the individual expressed amounts of multiple genes collectively, and in that the expression of multiple genes belonging to a specific gene family is analyzed collectively in order to identify a gene in that gene family the expression of which is characteristically promoted or inhibited in specific cells or tissue by computing the expressed amount thereof as an absolute value. To say that a gene's expression is characteristically promoted or inhibited signifies here that the gene's expression is greater or less to a physiologically significant degree than its expression in normal cells or tissue. In the present invention there are no particular limits on the gene families which are targeted, but they may be selected for example from the G protein-coupled receptor gene family, tyrosine kinase receptor gene family or ion channel gene family, or from gene families associated with transcription factors, transporters, protein kinases, protein phosphatases, proteases, heat shock proteins, ATPases, DNA-binding proteins or the like.

“Multiple genes” signifies 2 or more genes, and there is no particular upper limit as long as the number is practicable, but normally it is 2 to tens of thousands or preferably 2 to 1000 or more preferably 10 to 800 or ideally 10 to 300.

More specifically, the gene expression analysis of the present invention is carried out by bringing an mRNA sample which may contain multiple target mRNAs into contact with amplification reagents each of which contains a pair of primers corresponding to an individual target mRNA at multiple reaction sites or preferably the reaction sites of a reaction device having multiple reaction sites, performing an amplification reaction, and measuring produced amounts of the amplification products. This method is explained below.

(mRNA Sample)

One mode of the present invention is a method of analyzing gene expression by assaying the mRNA of multiple target genes, which may be contained in an mRNA sample. The “mRNA sample” here signifies a sample which contains mRNA and which is used for measuring the types and amounts of mRNA contained in the sample.

More specifically, the mRNA samples used in the present invention are samples used for analyzing the expressed level of specific genes in the sample, and are for example samples collected from the tissues of humans or other mammals (such as rats, mice, rabbits, sheep, pigs, cows, cats, dogs, monkeys and the like) or cultured cell strains thereof. Examples of such tissues include the brain, various parts of the brain (such as the olfactory bulb, amygdaloid nucleus, basal ganglia, hippocampus, thalamus, hypothalamus, subthalamic nucleus, cerebral cortex, medulla oblongata, cerebellum, occipital lobe, frontal lobe, temporal lobe, putamen, caudate nucleus, cerebral gland and substantia nigra), spinal cord, pituitary gland, stomach, pancreas, kidneys, liver, gonads, thyroid, gall bladder, bone marrow, adrenal gland, skin, muscles, lungs, digestive tract (such as the large and small intestine), blood vessels, heart, thymus, spleen, submandibular gland, peripheral blood, peripheral blood cells, prostate, testicles, testes, ovaries, placenta, uterus, bone, joints, skeletal muscle and the like. Using such an mRNA sample it is possible to analyze the expressed level of a target gene at the site from which the mRNA sample was collected by assaying target mRNA contained in the sample.

Namely, with the present invention it is possible to measure in one operation from an mRNA sample containing multiple mRNAs whether and how much target mRNA is produced. In particular, it is easy using an mRNA sample from a patient suffering from a particular disease to specify a gene (such as a GPCR gene) associated with that disease. In particular, when specifying G protein-coupled receptors, tyrosine kinase receptors and ion channel genes involved in multiple gene-associated diseases such as cancers which are thought to involve multiple genes, it is easy to specify the associated genes or proteins with the present invention because the expressed levels of all genes can be measured with one assay operation.

Assaying expressed gene levels and computing absolute values for expressed gene levels can be accomplished according to the target mRNA assay method described below.

(Reaction Device with Multiple Reaction Sites)

There are no particular limits on the number of multiple reaction sites as long as there are two or more, but normally there are 2 to tens of thousands or preferably 2 to 1000 or more preferably 10 to 800 or still more preferably 10 to 300.

In order to amplify target mRNAs which may be contained in an mRNA sample in the present invention, the sample is reacted all at once with individual amplification reagents each of which contains a pair of primers corresponding to a target mRNA. Consequently, in the present invention reaction of the mRNA sample with the individual amplification reagents is performed at individual reaction sites, preferably using a reaction device having multiple reaction sites. There are no limits on the composition and structure of the reaction device used in the present invention as long as it has two or more reaction sites to allow simultaneous reaction of the mRNA sample with the individual amplification reagents. Desirable examples of the amplification device used in the present invention include plates having multiple wells, reaction devices equipped with multiple glass slides and reaction devices equipped with multiple test tubes. Plates having multiple wells can be used by preference out of consideration of test space and operability. The plates can be selected depending on the number of primer pairs used, but commercially available 96-well and 384-well plates can be used by preference. However, a reaction device equipped with a desired number of reaction spites corresponding to the number of primer pairs can also be used. The method of the present invention can also be realized using 2 or more commercially available 96-well or 384-well plates.

(Amplification Reagents)

Each of the amplification reagents used in the present invention comprises for example a primer pair corresponding to a target mRNA, a probe corresponding to a target mRNA, DNA polymerase, buffer and the like.

As used here, “a primer pair corresponding to a target mRNA” signifies a pair of primers consisting of a first primer complementary to or substantially complementary to one chain of the exon region of a target gene sequence encoding a target mRNA, and a second primer complementary to or substantially complementary to the other chain of the exon region of the target gene sequence. In the present invention, the presence or absence of mRNA transcripted from target genes in an mRNA sample and the transcripted amounts thereof can be detected all at once by using at least two or more of such primer pairs. Consequently, the greater the number of primer pairs used, the more efficient the assay method of the present invention.

In the assay method of the present invention, there are no particular limits on the group of primer pairs used because it can be adjusted according to the number of target mRNAs, but for example it may consist of 10 to 800 primer pairs. In another mode of the present invention, the group of primer pairs used consists of 10 to 300 primer pairs. When the number of primer pairs is large, different sets of amplification reagents can be assigned to a number of plates (such as 2 to 10 plates), and the amplification reaction performed multiple times.

According to a preferred mode of the present invention, the target gene is a gene encoding a G protein-coupled receptor, tyrosine kinase receptor, ion channel or the like, and thus the target mRNA is gene mRNA belonging to the G protein-coupled receptor, tyrosine kinase receptor, ion channel or other family. In this case, an amplification reaction is performed by bringing amplification reagents each comprising a primer pair corresponding to gene mRNA belonging to a target G protein-coupled receptor, tyrosine kinase receptor, ion channel or other family into contact with- an mRNA sample in the respective reaction sites of a-reaction device, and the mRNA amplification products are assayed in order to measure the expressed amounts of genes belonging to the G protein-coupled receptor, tyrosine kinase receptor, ion channel and other families which were contained in the mRNA sample.

In a more preferred mode of the present invention, if all primer pairs are prepared corresponding to gene mRNA belonging to all known G protein-coupled receptor, tyrosine kinase receptor, ion channel and other families, and amplification reagents comprising the respective primer pairs are set in each reaction site, the degree to which gene mRNA belonging to any G protein-coupled receptor, tyrosine kinase receptor, ion channel or other family is produced in an mRNA sample can be assessed with a single assay operation. Of course, it is also possible when necessary to assign different sets of amplification reagents to different plates, and perform the assay reaction multiple times.

At present, the following G protein-coupled receptors (or genes) are known:
(1) Acetylcholine receptors: M₁; M₂; M₃; M₄; M₅
(2) Adenosine receptors: A₁; A_{2A}, A_{2B}, A₃
(3) Adrenoceptors: α1A; α1B; α1D; α2A; α2B; α2C; β1; β2; β3
(4) Angiotensin receptors: AT1; AT2
(5) Bombesin receptors: BB1; BB2; bb3
(6) Bradykinin receptors: B₁; B₂
(7) Calcitonin, amylin, CGRP and adrenomedullin receptors:
(8) Cannabinoid receptors: CB1; CB2
(9) Chemokine receptors: CCR1; CCR2; CCR3; CCR4; CCR5; CCR6; CCR7; CCR8; CCR9; CCR10; CXCR1; CXCR2; CXCR3; CXCR4; CXCR5; CX₃CRI; XCR1
(10) Cheniotactic receptors: C3a; C5a; fMLP
(11) Cholecystokinin and gastrin receptors: CCK₁; CCK₂
(12) Corticotropin-releasing factor receptors: CRF₁; CRF₂
(13) Dopamine receptors: D1; D2; D3; D4; D5
(14) Endothelin receptors: ET_{A}; ET_{B}
(15) Galanin receptors: GAL1; GAL2; GAL3
(16) Glutamate receptors: mglu₁; mglu₂; mglu₃; mglu₄; mglu₅; mglu₆; mglu₇; mglu₈
(17) Glycoprotein hormone receptors: FSH; LSH; TSH
(18) Histamine receptors: H₁; H₂; H₃; H₄
(19) 5-HT receptors: 5-HT_{1A}; 5-HT_{1B}; 5-HT_{1D}; 5-ht_{1B}; 5-ht_{1F}; 5-HT_{2A}; 5-HT_{2F}; 5HT_{2C}; 5-HT₃; 5-HT₄; 5-ht_{5A}; 5-ht_{5B}; 5-HT₆; 5-HT₇
(20) Leukotriene receptors: BLT, CysLT₁, CysLT₂
(21) Lysophospholipid receptors: edg1; edg2; edg3; edg4
(22) Melanocorlin receptors: MC₁; MC₂; MC₃; MC₄; MC₅
(23) Melatonin receptors: MT₁; MT₂; MT₃
(24) Neuropeptide Y receptors: Y₁; Y₂; Y₄; Y₅; Y₆
(25) Neurotension receptors: NTS1; NTS2
(26) Opioids: DOP; KOP; MOP; NOP
(27) P2Y receptors: P2Y₁; P2Y₂; P2Y₄; P2Y₆; P2Y₁₁; P2Y₁₂
(28) Peroxisome proliferator-activated receptors: PPAR-α; PPAR-β; PPAR-γ
(29) Prostanoid receptors: DP; FP; IP; TP; EP₁; EP₂; EP₃; EP₄
(30) Protease-activated receptors: PAR 1; PAR2; PAR3; PAR4
(31) Somatostatin receptors: sst₁; sst₂; sst₃; sst₄; sst₅
(33) Thyrotropin-releasing hormone receptors: TRH₁; TRH₂
(34) Urotensin-II receptors:
(35) Vasoactive intestinal peptide and pituitary adenylate cyclase activating peptide receptors: VPAC₁; VPAC₂; PAC₁
(36) Vasopressin and oxytocin receptors: V₁ₐ; V_{1b}; V₂; OT.

At present the following genes belonging to the tyrosine kinase receptor, ion channel gene and other families are known:
(37) Ion channels: Na⁺ channels (Type I; Type II/Type IIA; Type III; SCL11/NaG; PN1; NaCh6; NaDRG; SkM1/μ1, SkM2), K⁺ channels (Kv; EAG; KQT; IRK; ROMK; GIRK; K_{ATP} and the like), Ca²⁺ channels (α1G; α1E; α1S; α1C; α1D; α1B; α1A; IP3; ryanodine receptor and the like), Cl⁻ channels (GABA_{A}; GABA_{C}; glycine receptor; C1C0; C1C1; CFTR and the like), non-selective cation channels (nAChR; 5-HT₃; NMDA; AMPA; P_{2X}ATP; CNG and the like) and others
(38) Tyrosine kinase receptors: insulin receptors; EGF receptors and the like.

In another mode of the present invention, it is possible by using a group of primer pairs corresponding to a group of mRNAs of genes belonging to a G protein-coupled receptor, tyrosine kinase receptor or ion channel family to specify in one assay operation the genes belonging to the G protein-coupled receptor, tyrosine kinase receptor, ion channel or other family which are highly expressed among the genes belonging to the G protein-coupled receptor, tyrosine kinase receptor, ion channel or other family.

For example, targets for drug creation can be classified into three types: (1) proteins (GPCR, intranuclear receptors, ion channels and the like) which bind low molecular weight substances, (2) enzymes with catalytic activity towards low molecular weight or high molecular weight substrates, and (3) proteins that bind to macromolecules such as proteins, nucleic acids and polysaccharides. Following this classification, genes can be grouped as follows.

(1) Genes associated with the following proteins, which bind low molecular weight substances:

Adrenaline receptors; acetylcholine receptors; histamine receptors; dopamine receptors; serotonin receptors; glutamine receptors, endothelin receptors; vasopressin receptors; serotonin transporters; glucocorticoid receptors; estrogen receptors; Ca²⁺ channels; Na⁺ channels; Cl⁻ channels

(2) Genes associated with the following enzymes having catalytic activity towards low molecular weight or high molecular weight substrates:

HMG-CoA reductase; angiotensin convertase; thromboxane synthase; proton pump; aldose reductase; cyclooxygenase; phosphodiesterase; protein phosphorylase; protein dephosphorylase; HIV reverse transcriptase; fungal squalene epoxidase; DNA gyrase; beta-lactamase

(3) Genes associated with the following proteins, which bind to proteins, nucleic acids, polysaccharides and other macromolecules:

Insulin receptor; erythropoietin receptor; G-CSF receptor; growth hormone receptor; interferon receptor; growth factor receptor HER2; TNF receptor; platelet integrin GPIIb/IIIa

The pair of primers for amplifying the target mRNA can be easily designed by a person having ordinary skill in the field based on the sequence of the target GPCR gene (see for example Genome Res. Oct. 6, 1996(10): 986-94).

The method used to assay mRNA in the present invention can be used for a variety of purposes other than hGPCR function analysis. For example, a specific disease can be diagnosed by using a set of multiple amplification regents comprising pairs of primers, which detect mRNA produced by the known disease gene. Because the expressed level of each gene can be measured accurately by the present invention, it offers the advantage of more accurate diagnosis than prior methods.

Next, probes corresponding to the target mRNAs used in the present invention can be designed easily by a person having ordinary skill in the field based on the target sequence (see for example Genome Res. Oct. 6, 1996(10): 986-94). Suitable oligonucleotide probes for use in the present invention have a length of preferably about 15 to about 50 nucleotides or more preferable about 25 to about 35 nucleotides. The oligonucleotide probes can be labeled by the incorporation of a detectable chemical substance or the like by biochemical, immunochemical or chemical means. Useful labels include ³²P and other radioactive isotopes, fluorescamine, fluorescein isothiocyanate and other fluorescent substances, luminol, luciferin and other luminous substances, beta-galatosidase, peroxidase, alkaliphosphatase and other enzymes, biotin and antibodies and the like. Examples of DNA polymerase which can be used in the present invention include heat-resistant DNA polymerase having reverse transcription and 5′→3′ exonuclease activity, such as rTth DNA polymerase and the like.

Well-known or commercially available buffers can be used in the present invention (for example PE Biosystems buffer, see Genome Res. Oct. 6, 1996(10): 986-94).

(Amplification of mRNA)

In the analysis method of the present invention, target mRNA, which may be contained in an mRNA sample, is amplified using an amplification reagent comprising a primer pair for amplifying said target mRNA. In a preferred mode of the present invention, amplification of the target mRNA is accomplished using a conventional polymerase chain reaction (PCR) (see U.S. Pat. Nos. 4,683,195, 4,683,202 and 4,965,188 and the like).

mRNA can be amplified by reversely transcribing the target mRNA using for example a virus reverse transcriptase, and then amplifying the resulting cDNA. In a more preferred mode, mRNA is amplified using a reverse transcriptase polymerase chain reaction (RT-PCR) (see U.S. Pat. Nos. 5,310,652, 5,322,770, 5,561,058, 5,641,864, 5,693,517 and the like).

A variety of mRNA amplification methods other than the aforementioned polymerase chain reaction can be used in the present invention. Such amplification methods include for example strand-displacement amplification (U.S. Pat. No. 5,455,166 and the like), transcription-based amplification system (TAS) (U.S. Pat. Nos. 5,437,990, 5,409,818, 5,399,491 and the like) and self-sustained sequence replication (3SR) (WO 92/08800 and the like).

The conditions for these amplification reactions can be easily designed by a person having ordinary skill in the field according to the types of reagents used and the like.

(Methods of Assaying Target mRNA)

Next, the amount of the aforementioned mRNA amplification product produced is assayed in the analysis method of the present invention. Preferably the amplification product is assayed by a method employing a probe. In a preferred mode, it is assayed by a method employing a probe labeled with a fluorescent substance.

In a more preferred mode of the present invention, the target mRNA is assayed using the “TaqMan method” or “5′ nuclease assay method” (Proceedings of the National Academy of Sciences U.S.A., Vol. 88, pp. 7276-7280 (1991); U.S. Pat. Nos. 5,210, 015, 5,487,972, 5,804,375 and the like). In the TaqMan assay method, a probe labeled at the 5′-terminal is used. The 3′-terminal of this probe is also modified to prevent the probe from acting as a primer for DNA synthesis. Modification can be by addition of a phosphate base, fluorescent substance or the like to the end. Amplification of target mRNA is accomplished using DNA polymerase having 5′→3′ exonuclease activity, such as Tth DNA polymerase. The probe, which hybridizes with target mRNA downstream from the primer, is degraded by the 5′→3′ exonuclease activity of the DNA polymerase during the amplification reaction. Each time a new target region is amplified, the probe is degraded and label substance released. The amount of target mRNA can be measured indirectly by assaying this released label substance.

Well-known methods can be used to quantitatively detect the released label substance. In a preferred method, the probe is labeled at the 5′- and 3′-terminals with two fluorescent substances, one of which can quench the fluorescence of the other substance. The fluorescence of this probe is quenched by the interaction of the two fluorescent substances as the probe hybridizes with the template DNA, but it emits fluorescence as it is broken down by the 5′→3′ exonuclease activity of the DNA polymerase. As the amplification reaction progresses the fluorescence increases and this increase is monitored.

A sample containing target mRNA is assayed based on a “standard curve” which is prepared by amplifying a sample containing a previously known quantity of target mRNA. The standard curve is used to calculate the number of input copies, which are derived from signals emitted during amplification. Consequently, an unknown number of copies of a target sequence in a sample is estimated by calculating the number of copies which were determined in advance to emit a signal equivalent to what is observed. Next, the concentration of the target sequence in the sample can be calculated from the number of input copies determined before reaction and the size of the sample (see Kokai No. 2001-204483 and the like).

An internal standard can also be used to eliminate experimental errors. Assay methods based on amplification which use internal standards are disclosed in U.S. Pat. Nos. 5,219,717 and 5,476,774 and the like. Various methods are known for improving the specificity of the amplification reaction, such as that described for example in European Patent Application No. 0866,071 and the like.

(Primer Pair Kit and Assay Kit)

The present invention a also relates to a primer pair kit and mRNA assay kit for performing the method of the present invention.

The primer pair kit of the present invention is a kit comprising two or more pairs of primers each consisting of a first primer which is complementary or substantially complementary to one chain of the exon region of a target gene sequence and a second primer which is complementary or substantially complementary to the other chain of the exon region of a target gene sequence. The assay method of the present invention can be performed rapidly if such a kit is prepared beforehand. A preferred primer pair kit of the present invention has a pair of primers corresponding to mRNA transcripted from a G protein-coupled receptor protein gene. In one mode of the present invention, the primer pair kit comprises a group of primer pairs consisting of 10 to 800 primer pairs or 10 to 300 primer pairs.

In a more preferred mode of the present invention, an mRNA assay kit is provided wherein each of the reaction sites of a reaction device having multiple reaction sites is filled with an amplification reagent which comprises a primer pair corresponding to a target mRNA. Preferably, the target gene in this kit is a disease-associated gene. More preferably, the kit also comprises a fluorescent probe, and still more preferably it comprises Tth DNA polymerase. As necessary, these kits may also contain for example chemicals which catalyze synthesis of primer elongation products, substrate nucleoside triphosphate, means used for labeling (such as avidin-enzyme conjugate, enzyme substrate and pigment base for example if the label is avidin), suitable buffers for the PCR or hybridization reaction and the like.

(Means of Diagnosing Diseases and Drugs for Treating those Diseases)

Next, gene expression analysis can be performed according to the method of (30) above of the present invention using an mRNA sample taken from a patient, and a disease from which the patient suffers diagnosed by detecting the characteristic expression of a specific disease-associated gene. With the present invention, it is possible by collectively analyzing the expression of genes belonging to a specific disease-associated gene facility (such as a specific disease-associated GPCR gene family) to specify in one operation the gene out of multiple specific disease-associated genes whose expression is characteristic.

Consequently, drugs containing agonists, antagonists or antibodies to gene products of a gene specified in this way or DNA encoding such gene products are particularly useful for patients with such a diagnosis. In the present invention multiple genes with abnormal expression can be specified, and the expression levels of those genes can even be assayed accurately. As a result, multiple agonists, antagonists or antibodies can be selected as suitable for prescription to the patient, and the prescribed amounts can be adjusted according to the expressed level of the disease-associated gene. In other words, with the present invention it is possible to devise so-called tailor-made drugs, which are prescribed only for that individual patients.

More specifically, if the physiological function of a ligand cannot be relied upon because a particular receptor protein is reduced in the body of a patient (deficiency of the receptor protein), the action of the ligand can be adequately restored by (1) administering the receptor protein to the patient to replace the missing receptor protein, or (2) increasing the amount of receptor protein in the patient's body by either (i) inducing expression by administering DNA encoding the receptor protein of the present invention to the patient, or (ii) inducing expression by inserting DNA encoding the receptor protein of the present invention into target cells, and transplanting the cells into the patient.

The drug of the present invention is effective for preventing or treating diseases involving a specific gene, and is useful for example for preventing and/or treating central disorders (such as Alzheimer's disease, dementia, eating disorders and the like), endocrine disorders (such as hypertension, gonad dysfunction, thyroid dysfunction, pituitary dysfunction and the like), metabolic disorders (such as diabetes, abnormal lipid metabolism, hyperlipidemia and the like), cancer (such as non-small cell carcinoma, ovarian cancer, prostate cancer, stomach cancer, bladder cancer, breast cancer, cervical cancer, colon cancer, rectal cancer and the like) and others.

When the genetic product of a gene specified by the present invention (such as a receptor protein), an agonist, antagonist or antibodies thereto, or DNA encoding that gene is used as the aforementioned preventative and/or therapeutic agent, it can be prepared by conventional means.

When DNA (sometimes abbreviated below as the DNA of the present invention) is used as the aforementioned preventative or therapeutic agent, such DNA can be applied by ordinary means either alone or after insertion into a suitable vector such as a retrovirus vector, adenovirus vector, adenovirus-associated virus vector or the like. Such DNA can be administered with a gene gun or hydrogel catheter or other catheter, either as is or together with adjuvants for promoting intake.

For example, the drug used in the present invention can be used orally in the form of tablets with a sugar coating as necessary, capsules, elixir, microcapsules or the like, or parenterally as an injection of a suspension or sterile solution with water or another pharmacologically acceptable liquid or the like. For example, the drug of the present invention can be manufactured by mixing with physiologically acceptable known carriers, flavorings, excipients, vehicles, preservatives, stabilizers, binders and the like in the unit dose form required by generally accepted preparation practice. The amount of the active ingredient in such preparations is such as to provide an appropriate dose within the indicated range.

Additives which can be mixed into tablets, capsules and the like include for example gelatin, corn starch, tragacanth, gum arabic or other binders, crystal cellulose and other excipients, corn starch, gelatin, alginic acid or other swelling agents, magnesium stearate or other lubricants, sucrose, lactose, saccharin or other sweeteners, and peppermint, akamono oil, cherry or other flavorings. When the dispensing unit form is capsules, a liquid carrier such as fat or oil can be included in addition to the aforementioned types of ingredients. A sterile composition for injection may be formulated by ordinary means of preparation such as dissolving or suspending the active substance and sesame seed oil, coconut oil or another naturally-produced vegetable oil or the like in a vehicle such as injection water. Physiological saline for example or an isotonic solution containing glucose or another adjuvant (such as D-sorbitol, D-mannitol, sodium chloride or the like) can be used as the aqueous solution for injection, and suitable solubilizers such as alcohols (ethanol for example), polyalcohols (propylene glycol or polyethylene glycol for example), non-ionic surfactants (polysorbate 80™ or HCO-50 for example) may be included. Sesame seed oil, soybean oil or the like for example can be used as an oily liquid, and may be used in conjunction with a solubilizer such as benzyl benzoate, benzyl alcohol or the like.

The aforementioned preventative and/or therapeutic agent may also be combined with buffers (such as phosphate buffer or sodium acetate buffer), analgesics (such as benzalkonium chloride, procaine hydrochloride and the like), stabilizers (such as human serum albumin, polyethylene glycol and the like), preservatives (such as benzyl alcohol, phenol and the like), antioxidants and the like. The formulated injection liquid is normally packed in suitable ampoules.

Since the resulting preparation is stable and low toxic, it can be administered for example to humans and other mammals (such as rats, mice, rabbits, sheep, pigs, cows, cats, dogs, monkeys and the like).

The dosage of the preventative and/or therapeutic agent of the present invention differs depending on the subject of administration, organ, symptoms, method of administration and the like, but for oral administration it is normally about 0.1 mg to 100 mg or preferably about 1.0 to 50 mg or more preferably about 1.0 to 20 mg a day for example in the case of a cancer patient (weight 60 kg). For parenteral administration, the single administered dose differs depending on the subject of administration, organ, symptoms, method of administration and the like, but in the case of injection normally about 0.01 to 30 mg or preferably about 0.1 to 20 mg or more preferably about 0.1 to 10 mg a day for example should be administered by intravenous injection in the case of a cancer patient (weight 60 kg). In the case of other animals, the dose can be converted from the 60 kg dose.

The dose of the DNA of the present invention differs depending on the subject of administration, organ, symptoms and administration method, but for oral administration it is normally about 0.1 mg to 100 mg or preferably about 1.0 to 50 mg or more preferably about 1.0 to 20 mg a day for example in the case of a cancer patient (weight 60 kg). For parenteral administration, the single administered dose differs depending on the subject of administration, organ, symptoms, method of administration and the like, but for example in the case of injection normally about 0.01 to 30 mg or preferably about 0.1 to 20 mg or more preferably about 0.1 to 10 mg a day for example should be administered by intravenous injection in the case of a cancer patient (weight 60 kg). In the case of other animals, the dose can be converted from the 60 kg dose.

A novel use for a receptor protein comprising an amino acid sequence identical to or substantially identical to the amino acid sequence represented by SEQ ID NO: 9 for example which is obtained using the aforementioned gene expression analysis method of the present invention is explained below.

(Receptor Protein)

The receptor protein used in the present invention is a receptor protein (sometimes referred to hereunder as the receptor protein of the present invention) comprising an amino acid sequence identical or substantially identical to the amino acid sequence represented by SEQ ID NO: 9. The protein having the amino acid sequence represented by SEQ ID NO: 9 is the human-derived dJ287G14.2 receptor protein. The human-derived dJ287G14.2 receptor has an amino acid sequence identical to that of the protein described in WO 2001/18207.

The present inventors confirmed for the first time that a protein exhibiting an affinity for a sugar chains such as an asparagine-linked sugar chain or serine/threonine-linked sugar chain (such as concanavalin A (ConA), lentil lectin, pea lectin, Datura lectin, Maackia Amurensis lectin, phytohemagglutinin and other lectins for example) is one of the ligands of the dJ287G14.2 receptor. The receptor protein of the present invention may be a protein derived from any cells (such as retinal cells, spleen cells, nerve cells, glia cells, pancreatic beta cells, bone marrow cells, mesangial cells, Langerhans' cells, epidermal cells, epithelial cells, endothelial cells, fibroblasts, fibrocytes, muscle cells, fat cells, immune cells (such as macrophages, T cell, B cells, natural killer cells, mast cells, neutrophils, basophils, acidophils, monocytes or luekocytes), megakaryocytes, synovial cells, cartilage cells, bone cells, osteoblasts, osteoclasts, mammary gland cells, liver cells or interstitial cells, or precursor cells, stem cells or cancer cells of these cells (such as breast cancer cell lines (GI-101), colon cancer cell lines (CX-1, GI-112), lung cancer cell lines (LX-1, GI-117), ovarian cancer cells lines (GI-102), prostate cancer cell lines) or the like) of humans and other mammals (such as guinea pigs, rats, mice, rabbits, pigs, sheep, cows, monkeys and the like), or from any tissues wherein these cells are present, such as the brain, various parts of the brain (such as the olfactory bulb, amygdaloid nucleus, basal ganglia, hippocampus, thalamus, hypothalamus, subthalarnic nucleus, cerebral cortex, medulla oblongata, cerebellum, occipital lobe, frontal lobe, temporal lobe, putamen, caudate nucleus, cerebral gland and substantia nigra), spinal cord, pituitary gland, stomach, pancreas, kidneys, liver, gonads, thyroid, gall bladder, bone marrow, adrenal gland, skin, muscles, lungs, digestive tract (such as the large and small intestine), blood vessels, heart, thymus, spleen, submandibular gland, peripheral blood, peripheral blood cells, prostate, testicles, testes, ovaries, placenta, uterus, bone, joints, skeletal muscle and the like, or from blood cells or cultured cells thereof (such as MEL, M1, CTLL-2, HT-2, WEHI-3, HL-60, JOSK-1, K562, ML-1, MOLT-3, MOLT4, MOLT-10, CCRF-CEM, TALL-1, Jurkat, CCRT-HSB-2, KE-37, SKW-3, HUT-78, HUT-102, H9, U937, THP-1, HEL, JK-1, CMK, KO-812, MEG-01 and the like), or it may be a synthetic protein.

In these Specifications, a “substantially identical amino acid sequence” signifies an amino acid sequence having about 50% or greater or preferably about 60% or greater or more preferably about 70% or greater or even more preferably about 80% or greater or still more preferably about 90% or greater or most preferably about 95% or greater homology with the compared amino acid sequence.

A protein or the like comprising an amino acid sequence substantially identical to the amino acid sequence represented by SEQ ID NO: 9 for example and having substantially the same activity as a protein comprising the amino acid sequence represented by SEQ ID NO: 9 for example is preferred as the protein comprising an amino acid sequence substantially identical to the amino acid sequence represented by SEQ ID NO: 9.

“Substantially the same activity” refers for example to ligand binding activity, signal transmission action and the like. Substantially the same means that these activities are the same in character. Consequently, the ligand binding activity, signal transmission action or other activity is preferably equivalent (such as about 0.01 to 100 times or preferably about 0.5 to 20 times or more preferably about 0.5 to 2 times), but quantitative factors such as the degree of activity and the molecular weight of the protein may be different.

Ligand binding activity, signal transmission action and other activity can be measured according to well-known methods, and for example can be measured according to the ligand determination method and screening method described below.

Moreover, a protein comprising (1) an amino acid sequence having 1 or 2 or more (preferably about 1 to 30 or more preferably 1 to 10 or still more preferably several (1 to 5)) amino acids deleted from the amino acid sequence represented by SEQ ID NO: 9, SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 27, (2) an amino acid sequence having 1 or 2 or more (preferably about 1 to 30 or more preferably 1 to 10 or still more preferably several (1 to 5)) amino acids added to the amino acid sequence represented by SEQ ID NO: 9, SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 27, or (3) an amino acid sequence having 1 or 2 or more (preferably about 1 to 30 or more preferably 1 to 10 or still more preferably several (1 to 5) amino acids replaced by other amino acids in to the amino acid sequence represented by SEQ ID NO: 9, SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 27, or (4) an amino acid sequence which is a combination of these or the like can also be used as the receptor protein of the present invention.

Following peptide marking conventions, the left end of the receptor protein of the present specifications is the N terminus (amino terminus) and the right end is the C terminus (carboxyl terminus). The C terminus of the receptor protein of the present invention may be a carboxyl group (—COOH), carboxylate (—COO⁻), amide (—CONH₂) or ester (—COOR).

A methyl, ethyl, n-propyl, isopropyl or n-butyl or other C₁₋₆ alkyl group for example, a cyclopentyl, cyclohexyl or other C₃₋₈ cycloalkyl group for example, a phenyl, alpha-naphthyl or other C₆₋₁₂ aryl group for example, a benzyl, phenethyl or other phenyl-C₁₋₂ alkyl group for example or an alpha-naphthylmethyl or other alpha-naphthyl-C₁₋₂ alkyl group or other C₇₋₁₄ aralkyl group or else a pivaloyl oxymethyl group or the like widely used as an ester for oral use can be used as the R in the ester here.

When the receptor protein of the present invention has a carboxyl group (or carboxylate) somewhere other than the C terminus, it is included in the receptor protein of the present invention if the carboxyl group is amidified or esterified. The aforementioned ester of the C terminus or the like for example can be used as the ester in this case.

The aforementioned protein in which the amino group of the methionine residue of the N terminus is protected by a protective group (such as a formyl group, acetyl or other C₂₋₆ alkanoyl or other C₁₋₆ acyl group or the like), in which a glutamyl group produced by nicking of the N end in vivo is pyroglutaminated, or in which a substitutional group (such as an —OH, —SH, amino group, imidazole group, indole group, guanidino group or the like) on a side chain of an amino acid in the molecule is protected by a suitable protective group (such as a formyl group, acetyl or other C₂₋₆ alkanoyl or other C₁₋₆ acyl group or the like) is included as the receptor protein of the present invention, as are composite proteins such as so-called glycoproteins having bound sugar chains.

Specific examples of the receptor protein of the present invention include for example a human-derived dJ287G14.2 receptor protein consisting of the amino acid sequence represented by SEQ ID NO: 9, a mouse-derived dJ287G14.2 receptor protein consisting of an amino acid sequence represented by SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 27 and the like.

The mouse-derived dJ287G14.2 receptor protein consisting of the amino acid sequence represented by SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 27 is a novel protein.

The receptor protein or salt thereof of the present invention may be manufactured from the aforementioned human or mammalian cells or tissue by well-known methods of purifying receptor proteins, or may be manufactured by culturing the transformant described below which comprises DNA encoding the receptor protein of the present invention. It may also be manufactured according to the protein synthesis methods described below or corresponding methods.

In the case of manufacture from human or mammalian tissue or cells, the human or mammalian tissue or cells are first homogenized, then extracted with acid or the like, and the extract can be purified and isolated by a combination of chromatographic methods such as reverse-phase chromatography, ion exchange chromatography and the like.

(Partial Peptide of the Receptor Protein)

The partial peptide of the present invention (sometimes abbreviated hereunder as “partial peptide”) may be any peptide having a partial amino acid sequence of the receptor protein of the present invention, but for example of the molecules of the receptor protein of the present invention, sites which are exposed outside the cell membrane and have receptor binding activity substantially equivalent to that of the receptor protein of the present invention and the like are used.

Specifically, partial peptides of the receptor protein of the present invention having the amino acid sequence represented by SEQ ID NO: 9 are peptides which include a part analyzed by hydrophobic plot analysis as being an extracellular region (hydrophilic site). Peptides comprised in part of a hydrophobic site can be used in the same way. Peptides, which separately comprise individual domains, can be used, as can peptides of a part, which comprises multiple domains simultaneously.

As for the number of amino acids in the partial peptide of the present invention, a peptide having an amino acid sequence of at least 20 or more or preferably 50 or more or more preferably 100 or more out of the constituent amino acid sequence of the receptor protein of the present invention or the like is preferred.

Substantially identical amino acid sequences are amino acid sequences having about 50% or greater or preferably about 60% or greater or more preferably about 70% or greater or even more preferably about 80% or greater or still more preferably about 90% or greater or most preferably about 95% or greater homology with these amino acid sequences.

“Substantially equivalent receptor binding activity” has the same significance here as above. “Substantially equivalent receptor binding activity” is measured in the same way here as above.

The partial peptide of the present invention may also have 1 or two or more (preferably about 1 to 10 or preferably several (1 to 5)) amino acids deleted from the aforementioned amino acid sequence, 1 or two or more (preferably 1 to 20 or more preferably 1 to 10 or still more preferably several (1 to 5)) amino acids added to the aforementioned amino acid sequence, or 1 or 2 or more (preferably about 1 to 10 or more preferably several or still more preferably 1 to 5) amino acids replaced by other amino acids in the aforementioned amino acid sequence.

The C terminus of the partial peptide of the present invention may be a carboxyl group (—COOH), carboxylate (—COO⁻), amide (—CONH₂) or ester (—COOR).

As with the receptor protein of the present invention described above, the partial peptide of the present invention also includes those in which the amino group of the methionine residue of the N terminus is protected by a protective group, those in which a Gln produced by nicking of the N end in vivo is pyroglutaminated, and those in which a substitutional group on a side chain of an amino acid in the molecule is protected by a suitable protective group, as well as so-called glycopeptides having bound sugar chains and the like.

Examples of salts of the receptor protein or partial peptide thereof of the present invention include physiologically allowable salts with acids and bases. Physiologically allowable acid-added salts are particularly desirable. Such salts, which can be used, include salts with inorganic acids (such as hydrochloric acid, phosphoric acid, hydrobromic acid, sulfuric acid) and salts with organic acids (such as acetic acid, formic acid, propionic acid, fumaric acid, maleic acid, succinic acid, tartaric acid, citric acid, malic acid, oxalic acid, benzoic acid, methanesulfonic acid, benzenesulfonic acid) and the like.

The partial peptide or salt thereof of the present invention can be manufactured by well-known methods of peptide synthesis or by nicking the GPCR of the present invention with a suitable peptidase. Peptide synthesis may be for example by either solid-phase or liquid-phase synthesis. That is, a partial peptide or amino acids, which may constitute the GPCR of the present invention, can be condensed with the residual part, and the target peptide manufactured by removing the protective groups if the product has protective groups. Conventional methods of condensing and removing protective groups include those described in a) through e) below.
a) M. Bodanszky and M. A. Ondetti, Peptide Synthesis, Interscience Publishers, New York (1966)
b) Schroeder and Luebke, The Peptide, Academic Press, New York (1965)
c) Izumiya, Nobuo et al, Fundamental and Experimental Peptide Synthesis, Maruzen (1975)
d) Yashima, Haruaki and Sakakibara, Shunpei, Biochemical Experimental Course 1. Protein Chemistry IV, 205 (1977)
e) Yashima, Haruaki ed, Drug Development Vol.14. Peptide Synthesis, Hirokawa Shoten

After the reaction the partial peptide of the present invention can be purified and isolated by a combination of solvent extraction, distillation, column chromatography, liquid chromatography, recrystallization and the like for example. If the partial peptide obtained by these methods is in free form, it can be converted to an appropriate salt by well-known methods, or conversely if it is obtained as a salt it can be converted to free form by well-known methods.

(Polynucleotide)

A polynucleotide encoding the receptor protein of the present invention may be any that contains a nucleotide sequence (DNA or RNA, preferably DNA) encoding the receptor protein of the present invention. This polynucleotide may be DNA, mRNA or other RNA encoding the receptor protein of the present invention, and may be double-stranded or single-stranded. If double-stranded it may be double-stranded DNA, double-stranded RNA or a DNA:RNA hybrid. If single-stranded, it may be a sense strand (namely a coding strand) or an antisense strand (namely a non-coding strand).

A polynucleotide encoding the receptor protein of the present invention can be used to assay mRNA of the receptor protein of the present invention by the well-known methods described in Jikken Igaku Zokan, “A new PCR and its application” 15(7), 1997 or methods conforming thereto for example.

DNA encoding the receptor protein of the present invention may be genome DNA, genome DNA library, cDNA derived from the aforementioned cells or tissue, cDNA library derived from the aforementioned cells or tissue or synthetic DNA. The vector used for the library may be a bacteriophage, plasmid, cosmid, phagemid or the like. Total RNA or an mRNA fraction prepared from the aforementioned cells or tissue can be amplified directly by a reverse transcriptase polymerase chain reaction (abbreviated hereunder as RT-PCR).

Specific examples of DNA encoding the receptor protein of the present invention include for example DNA containing a nucleotide sequence represented by SEQ ID NO: 8, SEQ ID NO: 10, SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28, or DNA encoding a receptor protein having DNA which hybridizes under highly stringent conditions with DNA containing a nucleotide sequence represented by SEQ ID NO: 8, SEQ ID NO: 10, SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28, and having substantially the same activity (such as ligand binding activity, signal transmission action or the like) as a receptor protein containing the amino acid sequence represented by SEQ ID NO: 9.

DNA or the like containing a nucleotide sequence having about 70% or greater or preferably about 80% or greater or more preferably about 90% or greater or most preferably about 95% or greater homology with a nucleotide sequence represented by SEQ ID NO: 8, SEQ ID NO: 10, SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28 for example can be used as DNA which hybridizes under highly stringent conditions with DNA having a nucleotide sequence represented by SEQ ID NO: 8, SEQ ID NO: 10, SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28.

Hybridization can be performed by well-known methods or methods conforming thereto, such as for example the method described in Molecular Cloning 2^{nd} (J. Sambrook et al., Cold Spring Harbor Lab. Press, 1989). When using a commercial library, the methods described in the attached manual may be followed. More preferably, highly stringent conditions can be followed.

These highly stringent conditions are for example a sodium concentration of about 19 to 40 mM or preferably 19 to 20 mM and a temperature of about 50 to 70° C. or preferably about 60 to 65° C. A sodium concentration of about 19 mM and a temperature of about 65° C. are particularly desirable.

The meaning of “a polynucleotide containing part of the nucleotide sequence of DNA encoding the receptor protein or part of a nucleotide sequence complementary to that DNA” encompasses not only DNA encoding a partial peptide of the present invention as described below, but also RNA.

According to the present invention, an antisense polynucleotide (nucleic acids) capable of inhibiting the replication or expression of the receptor protein gene can be designed and synthesized based on the nucleotide sequence information for DNA encoding the cloned or determined receptor protein. Such a polynucleotide (nucleic acids) can hybridize with the RNA of the receptor protein gene, and can either inhibit the synthesis or function of such RNA or else regulate and control expression of the receptor protein gene by interacting with the receptor protein-associated RNA. Polynucleotides complementary to selected sequences of receptor protein-associated RNA and polynucleotides which can hybridize specifically with receptor protein-associated RNA are useful for regulating and controlling expression of the receptor protein gene both in vivo and in vitro, and are useful for treating and diagnosing disease. The phrase “corresponds to” signifies having homology or being complementary to a nucleotide, nucleotide sequence or specific sequence of nucleic acids including a gene. What “corresponds” between a nucleotide, nucleotide sequence or nucleic acids and a peptide (protein) is normally amino acids of a peptide (protein) under control induced by the sequence of a nucleotide (nucleic acids) or its complement. The 5′ hairpin loop, 5′ 6-base pair lipid, 5′ non-translation region, polypeptide translation initiation codon, protein coding region, ORF translation termination codon, 3′ non-translation region, 3′ palindrome region and 3′ hairpin loop of the receptor protein gene can be selected as desirable target regions, but so can any region of the receptor protein gene.

Methods which can be used to clone DNA which completely encodes the receptor protein or partial peptide thereof of the present invention (sometimes abbreviated together below as the receptor protein of the present invention) are to amplify it by PCR using synthetic DNA primers having a partial nucleotide sequence of the receptor protein of the present invention, or to select it by hybridization with DNA incorporated into a suitable vector and labeled with either synthetic DNA or a DNA fragment encoding some or all regions of the receptor protein of the present invention. Hybridization can be accomplished for example by the method described in Molecular Cloning 2^{nd} (J. Sambrook et al., Cold Spring Harbor Lab. Press, 1989) or the like. When a commercial library is used, the methods described in the attached manual can be employed.

(Antisense Polynucleotide)

The relationship between the target nucleic acids and a polynucleotide complementary to at least part of the region of interest can be called an “antisense” relationship of a polynucleotide, which can hybridize with the object of interest. Examples of antisense polynucleotides include polynucleotides containing 2-deoxy-D-ribose, polynucleotides containing D-ribose, other types of polynucleotides which are N-glycosides of pyrimidine bases or purines, and other polymers having non-nucleotide frameworks (such as commercial proteins, nucleic acids and synthetic sequence-specific nucleic acid polymers) as well as other polymers containing special binding (when such polymers contain nucleotides which are arranged so as to allow base pairings and base attachments such as are found in DNA and RNA). These may be double-stranded DNA, single-stranded DNA, double-stranded RNA, single-stranded RNA or DNA:RNA hybrids, and may be unmodified polynucleotides (or unmodified oligonucleotides) or those with conventional modifications added, such as those having labels familiar in the field, those having caps, those that are methylated, those having one or more natural nucelotides replaced by analogues, those having intermolecular nucleotide modifications such as those with non-charged bonds (such as methylphosphonate, phosphotriester, phosphoramidate, carbamate or the like) and those having bonds with charge or sulfur-containing bonds (such as phosphorothioate, phosphorodithioate and the like) including for example proteins (nucleases, nuclease inhibitors, toxins, antibodies, signal peptides, poly-L-lysine and the like), sugars (such as monosaccharides and the like) and others with side-chain bases, those with intercalate bonds (such as acridine and psoralen), those with chelate compounds (such as metals, metals with radioactivity, boron, oxidizing metals and the like), those containing alkylating agents, or those having modified bonds (such as alpha anomer type nucleic acids). Here “nucleosides,” “nucleotides” and “nucleic acids” may include not only those with purine or pyrimidine bases, but those with other modified heterocyclic bases. These modified bases may include methylated purine and pyrimidine, acylated purine and pyrimidine and other heterocyclic structures. Modified nucleotides may also have their sugar parts modified, and for example one or more hydroxyl groups may be replaced by halogens or aliphatic groups, or converted to ether, amine or other functional groups.

The antisense polynucleotide (nucleic acids) of the present invention is RNA, DNA or modified nucleic acids (RNA, DNA). Specific examples of modified nucleic acids include sulfur derivatives or thiophosphate derivatives of nucleic acids, and those with resistance to degradation of polynucleoside amide and olignucleoside amide, but they are not limited by these examples. The antisense nucleic acids of the present invention can preferably be designed according to the following principles. Namely, the antisense nucleic acids within the cell are made more stable, the cell permeability of the antisense nucleic acids is enhanced, affinity for the target sense chain is increased, and toxicity of the antisense nucleic acids is reduced if they have toxicity.

Many such modifications are known in the field, and are disclosed for example in J. Kawakami et al., Pharm Tech Japan Vol. 8, pp 247, 1992 and Vol. 8, pp 395, 1992; S. T. Crooke et al. ed., Antisense Research and Applications, CRC Press, 1993 and the like.

The antisense nucleic acids of the present invention may be altered or may contain modified sugars, bases or bonds, or they may be provided in a special form such as a liposome or microsphere in a form more suitable for gene therapy or with additions. Examples of such additions which can be used include polylysine and other polycations which serve to neutralize the charge of phosphate group structures, and lipids (such phospholipids, cholesterol and the like) which are hydrophobic and improve interaction with the cell membrane and increase incorporation of nucleic acids. Desirable lipids for addition include cholesterol and its derivatives (such as cholesteryl chloroformate and cholic acid). These may be attached to the 3′-terminal or 5′-terminal of the nucleic acids, and may be attached via bases, sugars or intermolecular nucleoside bonds. Other groups include the capping groups, which are positioned specifically at the 3′-terminal or 5′-terminal of the nucleic acids and serve to prevent degradation by exonucleases, Rnases and other nucleases. Examples of such capping groups include protective groups of hydroxyl groups known in the field, including polyethylene glycol, tetraethylene glycol and other glycols, but they are not limited to these examples.

The inhibitory activity of antisense nucleic acids can be studied using a transformant of the present invention, an in vivo or in vitro gene expression system of the present invention or an in vivo or in vitro translation system of the receptor protein. The nucleic acids can be applied to cells by a variety of conventional methods.

Since the antisense polynucleotide of the present invention can suppress the function of the protein of the present invention or the polynucleotide of the present invention (such as DNA) in vivo, it can be used for example as a preventative and/or therapeutic drug for disorders associated with dysfunction of the receptor protein of the present invention. Moreover, since the antisense polynucleotide of the present invention can also be used as a diagnostic oligonucleotide probe for investigating the presence and expression of the DNA of the present invention in tissue and cells, it can be used for diagnosing disorders associated with dysfunction of the receptor protein of the present invention.

(DNA Encoding Partial Peptide)

The DNA encoding the partial peptide of the present invention may be any that contains a nucleotide sequence encoding the partial peptide of the present invention. It may be genome DNA, genome DNA library, cDNA derived from the aforementioned cells or tissue, cDNA library derived from the aforementioned cells or tissue, or synthetic DNA. The vector used for the library may be a bacteriophage, plasmid, cosmid, phagemid or the like. An mRNA fraction prepared from the aforementioned cells or tissue can be amplified directly by a reverse transcriptase polymerase chain reaction (abbreviated hereunder as RT-PCR).

Specifically, the following for example can be used as DNA encoding the partial peptide of the present invention:

(1) DNA having a partial nucleotide sequence of DNA containing a nucleotide sequence represented by SEQ ID NO: 8, SEQ ID NO: 10, SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28;

(2) DNA having a partial nucleotide sequence of DNA encoding a protein which has DNA which hybridizes under highly stringent conditions with DNA containing a nucleotide sequence represented by SEQ ID NO: 8, SEQ ID NO: 10, SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28 and which has substantially the same activity (such as ligand binding activity, signal transmission action or the like) as a protein peptide containing the amino acid sequence represented by SEQ ID NO: 9.

DNA or the like containing a nucleotide sequence having about 70% or greater or preferably about 80% or greater or more preferably about 90% or greater or most preferably about 95% or greater homology with a nucleotide sequence represented by SEQ ID NO: 8, SEQ ID NO: 10, SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28 for example can be used as DNA which hybridizes under highly stringent conditions with DNA containing a nucleotide sequence represented by SEQ ID NO: 8, SEQ ID NO: 10, SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28.

(Antibodies)

Antibodies to the receptor protein or partial peptide or salt thereof of the present invention may be either polyclonal antibodies or monoclonal antibodies as long as they are capable of recognizing the receptor protein or partial peptide or salt thereof of the present invention or cells or tissue containing the receptor protein of the present invention.

Antibodies to the receptor protein or partial peptide or salt thereof of the present invention (sometimes abbreviated hereunder as the receptor protein or the like of the present invention) can be manufactured according to well-known methods of manufacturing antibodies or antiserum, using the receptor protein or the like of the present invention as the antigen.

(Preparation of Monoclonal Antibodies)

(a) Preparation of Monoclonal Antibody-Producing Cells

The receptor protein or the like of the present invention is administered to mammals at a site where antibody production from administration as possible, either by itself or together with a carrier or diluent. Complete Freund's adjuvant or incomplete Freund's adjuvant can be administered to enhance antibody productivity during administration. Administration is normally once every 2 to 6 weeks, for a total of about 2 to 10 administrations. The mammals used may be monkeys, rabbits, dogs, guinea pigs, mice, rats, sheep or goats for example, with mice and rats being preferred.

To produce monoclonal antibody-producing cells, individuals with confirmed antibody titer are selected from warm-blooded animals (such as mice) which were immunized with the antigen, spleens or lymph nodes are removed 2 to 5 days after the final immunization, and antibody-producing cells contained therein can be fused with myeloma cells to prepare a monoclonal antibody-producing hybridoma. Measurement of antibody titer in antiserum can be accomplished for example by first reacting the labeled receptor protein or the like described below with antiserum, and then measuring the activity of label bound to the antibodies. Fusing can be accomplished by known methods such as the method of Kohler and Milstein (Nature 256, p. 495 (1975)). Examples of fusion promoters include polyethylene glycol (PEG), sendai virus and the like, but preferably PEG is used.

Examples of myeloma cells include NS-1, P3U1, SP2/0 and the like, but preferably P3U1 is used. The preferred ratio of number of antibody-producing cells (spleen cells) to number of myeloma cells used is about 1:1 to 20: 1, and cell fusion can be performed efficiently if PEG (preferably PEG1000 to PEG6000) is added at a concentration of about 10 to 80%, and the cells incubated for about 1 to 10 minutes at about 20 to 40° C. or preferably about 30 to 37° C.

Various methods can be used to screen the monoclonal antibody-producing hybridoma, and possible methods include for example a method of adding hybridoma culture supernatant to a solid phase (such as a microplate) on which the receptor protein or another antigen has been adsorbed either directly or together with a carrier, then adding protein A or anti-immunoglobulin antibodies (anti-mouse immunoglobulin antibodies are used if the cells used in cell fusion are mouse cells) labeled with a radioactive substance or enzyme, and detecting monoclonal antibodies bound to the solid phase, or a method of adding hybridoma culture supernatant to a solid phase on which anti-immunoglobulin antibodies or protein A have been adsorbed, then adding receptor protein or the like labeled with a radioactive substance or enzyme, and detecting monoclonal antibodies bound to the solid phase.

Well-known methods or methods conforming thereto can be used for selection of monoclonal antibodies, which is normally accomplished in an animal cell medium or the like to which HAT (hypoxanthine, aminopterin, thymidine) has been added. The medium for selection and breeding can be any medium in which the hybridoma can grow. For example, RPMI 1640 medium containing 1 to 20% or preferably 10 to 20% fetal calf serum, GIT medium (Wako Pure Chemical Industries, Ltd.) containing 1 to 10% fetal calf serum or serum-free medium for hybridoma culture (SFM-101, Nissui Pharmaceutical Co., Ltd.) or the like can be used. The culture temperature is normally 20 to 40° C. or preferably about 37° C. Culture time is normally 5 days to 3 weeks or preferably 1 week to 2 weeks. Culture can normally be performed under 5% carbon dioxide. The antibody titer of the hybridoma culture supernatant can be measured in the same way as the aforementioned measurement of antibody titer in antiserum.

(b) Purification of Monoclonal Antibodies

Isolation and purification of monoclonal antibodies can be accomplished in the same way as ordinary isolation and purification of polyclonal antibodies using methods of isolating and purifying immunoglobulin (for example, salting out, alcohol precipitation, isoelectric point precipitation, electrophoresis, adsorption-desorption with an ion exchanger (such as DEAE), ultracentrifugation, gel filtration, or a specific purification method in which antibodies are obtained by collecting them alone with an antigen-bound solid phase or an active adsorber such as protein A or protein G, and then dissociating the bonds).

(Preparation of Polyclonal Antibodies)

The polyclonal antibodies of the present invention can be manufactured by well-known methods or methods conforming thereto. For example, they can be manufactured by creating a composite of an immunogen (protein of the present invention or other antigen) and a carrier protein, immunizing mammals as in the manufacture of monoclonal antibodies described above, collecting material containing antibodies to the receptor protein or the like of the present invention from the immunized animals, and isolating and purifying the antibodies.

For the composite of immunogen and carrier protein used to immunize the mammals, the type of carrier protein and proportions of carrier and hapten may be any type and any proportions as long as antibodies are produced efficiently in response to the immunized hapten linked to the carrier, but for example a method can be used in which bovine serum albumin, bovine thyroglobulin, keyhole limpet hemocyanin or the like is coupled at a weight ratio of about 0.1 to 20 or preferably about 1 to 5 units per 1 unit of hapten.

Various condensing agents can be used for coupling the hapten and the carrier, but glutaraldehyde and active ester reagents containing carbodiimide, maleimide active ester, thiol groups and dithiopyridil groups can be used.

The condensed product is administered to warm-blooded animals either alone or together with a carrier or diluent at a site where antibodies can be produced. Complete Freund's adjuvant or incomplete Freund's adjuvant can be administered to enhance antibody productivity during administration. Administration is normally once every 2 to 6 weeks, for a total of about 3 to 10 administrations.

The polyclonal antibodies can be collected from the blood, abdominal fluid or the like of mammals immunized as described above, preferably from the blood.

Polyclonal antibody titer in antiserum can be measured in the same way as the measurement of antibody titer in serum described above. The polyclonal antibodies can be isolated and purified in the same way as the isolation and purification of monoclonal antibodies described above using methods of isolating and purifying immunoglobulin.

(Uses for Receptor Protein, DNA and the Like)

A protein showing affinity for an asparagine-linked sugar chain or a serine/threonine-linked sugar chain (such as concanavalin A, lentil lectin, pea lectin, datura stramonium lectin, sophora japonica lectin, phytohemagglutinin or another lectin or the like) is one ligand for the receptor protein of the present invention.

Consequently, the receptor protein of the present invention, the polynucleotide encoding therefor (sometimes abbreviated hereunder as the polynucleotide of the present invention), antibodies to the receptor protein of the present invention (sometimes abbreviated hereunder as antibodies of the present invention), the antisense polynucleotide to the DNA of the present invention (sometimes abbreviated hereunder as the antisense polynucleotide of the present invention) and the like have the following uses.

(1) Preventative and/or Therapeutic Agents for Disorders Associated with Dysfunction of the Receptor Protein of the Present Invention

Since expression of the receptor protein of the present invention is seen in the prostate (cancer), placenta and liver, a) the receptor protein of the present invention or (b) a polynucleotide encoding the receptor protein of the present invention (such as DNA) can be used as a drug such as a birth inducer or a preventative and/or therapeutic agent for dysfunction of the receptor protein of the present invention, particularly prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency or calculus.

For example, if the physiological function of a ligand cannot be relied upon because a particular receptor protein is reduced in the body of a patient (deficiency of the receptor protein), the action of the ligand can be adequately restored by (1) administering the receptor protein to the patient to replace the missing receptor protein, or (2) increasing the amount of receptor protein in the patient's body by either (i) inducing expression by administering DNA encoding the receptor protein of the present invention to the patient, or (ii) inducing expression by inserting DNA encoding the receptor protein of the present invention into target cells and transplanting the cells into the patient. In other words, a polynucleotide encoding the receptor protein of the present invention is useful as safe, low-toxicity birth inducer or preventative and/or therapeutic agent for dysfunction of the receptor protein, particularly prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency and calculus.

When the receptor protein or polynucleotide encoding therefor of the present invention is used as the aforementioned drug, it can be prepared according to ordinary means.

(2) Diagnostic Agent and Diagnostic Method Employing the Polynucleotide or Antisense Polynucleotide of the Present Invention

Since the polynucleotide (such as DNA) and antisense polynucleotide (such as antisense DNA) of the present invention can be used as probes to detect abnormalities (gene abnormalities) in DNA or mRNA encoding the receptor protein or partial peptide thereof of the present invention in humans or mammals (such as rats, mice, rabbits, sheep, pigs, cows, cats, dogs, monkeys and the like), they are useful as gene diagnosis agents for detecting damage, mutations or reduced expression of that DNA or mRNA, or increase, over-expression or the like of that DNA or mRNA.

The aforementioned gene diagnosis using the polynucleotide or antisense polynucleotide of the present invention can be accomplished for example by well-known Northern hybridization or PCR-SSCP methods (Genomics 5, 874-879 (1989); Proceedings of the National Academy of Sciences of the United States of America 86, 2766-2770 (1989)) or the like.

For example, if reduced expression or over-expression of the receptor protein of the present invention is detected by Northern hybridization, it is possible to diagnose a high likelihood of a disorder stemming from dysfunction or over-expression of the receptor protein of the present invention for example, particularly prostate cancer, other cancers (non-small cell carcinoma, ovarian cancer, stomach cancer, bladder cancer, breast cancer, cervical cancer, colon cancer, rectal cancer large intestinal cancer), prostatomegaly, male gonad dysfunction, infertility, premature birth, endometriosis, cirrhosis of the liver, hepatitis, hepatic insufficiency or calculus, or a high likelihood of such a disorder occurring in the future.

(3) Drugs Containing the Antisense Polynucleotide of the Present Invention

The antisense polynucleotide of the present invention can be used as a birth inducer or as a preventative and/or therapeutic agent for disorders caused by over-expression or the like of the receptor protein of the present invention (such as prostate cancer and other cancers (non-small cell carcinoma, ovarian cancer, stomach cancer, bladder cancer, breast cancer, cervical cancer, colon cancer, rectal cancer large intestinal cancer), prostatomegaly, male gonad dysfunction, infertility, premature birth, endometriosis, cirrhosis of the liver, hepatitis, hepatic insufficiency, calculus and other disorders).

For example, when this antisense polynucleotide is used it can be used alone or first inserted into a suitable vector such as a retrovirus vector, adenovirus vector, adenovirus-associated virus vector or the like and applied by ordinary means. The antisense polynucleotide can be prepared alone or together with adjuvants for promoting ingestion or other physiologically acceptable carriers, and administered with a gene gun or a catheter such as a hydrogel catheter.

Moreover, the antisense polynucleotide can also be used as a diagnostic oligonucleotide probe for investigating the presence and expression of the DNA of the present invention in cells or tissue.

(4) Method of Screening Compounds, which Alter the Expressed Amount of the Receptor Protein or Partial Peptide thereof of the Present Invention

When used as a probe, the DNA of the present invention can be used for screening compounds, which alter the expressed amount of the receptor protein or partial peptide thereof of the present invention.

Namely, the present invention provides for example a method of screening compounds which alter the expressed amount of the receptor protein or partial peptide thereof of the present invention by measuring the amount of mRNA of the receptor protein or partial peptide thereof of the present invention contained in for example (i) (1) blood, (2) specific organs or (3) tissue or cells isolated from organs of non-human mammals or in (ii) a transformant or the like.

Specifically, the amount of mRNA of the receptor protein or partial peptide thereof of the present invention is measured as follows.

(i) A drug (such as an anti-cancer drug) or physical stress (such as immersion stress, electric shock, brightness contrast or low temperature) or the like is applied to normal or disease-model non-human mammals (such as mice, rats, rabbits, sheep, pigs, cows, cats, dogs, monkeys or the like, or more specifically cancer-carrying mice), and after a fixed amount of time blood or specific organs (such as brains, lungs, large intestines, prostate glands or the like) or tissue or cells isolated from organs are obtained.

mRNA of the receptor protein or partial peptide thereof of the present invention contained in the resulting cells can be extracted from the cells or the like by ordinary methods and assayed using a method such as TaqMan PCR for example, and can also be analyzed by Northern blotting using well-known means.

(ii) A transformant expressing the receptor protein or partial peptide thereof of the present invention can be prepared according to the methods described above, and mRNA of the receptor protein or partial peptide thereof of the present invention contained in the transformant assayed and analyzed in the same way.

Compounds, which alter the expressed amount of the receptor protein or partial peptide thereof of the present invention, can be screened by:

(i) Administering a test compound to normal or disease model non-human mammals a fixed time before (30 minutes to 24 hours before, preferably 30 minutes to 12 hours before, more preferably 1 hour to 6 hours before) or a fixed time after (30 minutes to 3 days after, preferably 1 hour to 2 days after, more preferably 1 hour to 24 hours after) application of a drug or physical stress or simultaneously with the drug or physical stress, and assaying and analyzing the amount of mRNA of the receptor protein or partial peptide thereof of the present invention contained in cells a fixed time after administration (30 minutes to 3 days, preferably 1 hour to 2 days, more preferably 1 hour to 24 hours);

(ii) Mixing a test compound into the medium when culturing the transformant by ordinary methods, and assaying and analyzing the amount of mRNA of the receptor protein or partial peptide thereof of the present invention contained in the transformant after a fixed culture time (1 day to 7 days, preferably 1 day to 3 days, more preferably 2 days to 3 days).

A compound or salt thereof obtained using the screening method of the present invention is a compound having the action of altering the expressed amount of the receptor protein or partial peptide thereof of the present invention, and specifically it is a compound which either (a) increases the expressed amount of the receptor protein or partial peptide thereof of the present invention, thus augmenting cell stimulus activity via the receptor, or (b) decreases the expressed amount of the receptor protein or partial peptide thereof of the present invention, thus weakening the cell stimulus activity.

Examples of cell stimulus activity include (1) arachidonic acid release, (2) acetylcholine release, (3) intercellular Ca release, (4) intercellular cAMP production, (5) intercellular cGMP production, (6) inositolphosphoric acid production, (7) cell membrane potential fluctuation, (8) phosphorylation of intercellular proteins (such as MAP kinase), (9) activation of c-fos, (10) reduction of pH, (11) activation of Rho, Rac, Ras and other low molecular weight G proteins, and (12) activation of reporter genes (such as luciferase and the like) attached downstream from transcription factor CRE (cAMP responsive element), AP1, NFAT, SRE (serum responsive element) or the like, with promotion of intercellular cAMP production and the like being especially desirable.

Examples of the compound include peptides, proteins, non-peptidergic compounds, synthetic compounds, fermentation products and the like, and this compounds may be either a novel compound or a well-known compound.

Such compounds, which augment cell stimulus activity, are useful as safe, low toxic drugs for augmenting the physiological activity of the receptor protein or the like of the present invention.

Such compounds, which weaken cell stimulus activity, are useful as safe, low toxic drugs for depressing the physiological activity of the receptor protein or the like of the present invention.

As explained above, one ligand of the receptor protein of the present invention is a protein (such as a lectin) showing an affinity for a sugar chain. Consequently, a compound obtained by the aforementioned screening method which alters the expressed amount of the receptor protein of the present invention can be used as a birth inducer or as a preventative and/or therapeutic agent for prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency, calculus or the like.

(5) Preventative and/or Therapeutic Drug for Various Disorders Containing a Compound which Alters the Expressed Amount of the Receptor Protein or Partial Peptide thereof of the Present Invention

As mentioned above, the receptor protein of the present invention is thought to serve some important role in the body in central function or the like for example. Consequently, a compound which alters the expressed amount of the receptor protein or partial peptide thereof of the present invention can be used as a preventative and/or therapeutic agent for disorders associated with dysfunction of the receptor protein of the present invention.

When this compound is used as a preventative and/or therapeutic agent for disorders associated with dysfunction of the receptor protein of the present invention, it can be formulated according to ordinary means.

(6) Screening Method and Screening Kit for Compounds (Agonists, Antagonists and the like) which Alter the Binding Properties of the Receptor Protein of the Present Invention with a Ligand

By using the receptor protein or the like of the present invention or by constructing a system expressing a recombinant receptor protein or the like and using a receptor binding assay system employing this expression system, it is possible to efficiently screen for compounds (such as peptides, proteins, non-peptidergic compounds, synthetic compounds, fermentation products and the like) or salts thereof which alter the binding properties of the receptor protein or the like of the present invention with a ligand.

As described above, one example of a ligand for the receptor protein of the present invention is a protein (such as a lectin) showing an affinity for a sugar chain.

Moreover, a compound or salt thereof, which alters the binding properties of a protein showing affinity for a sugar chain with the receptor protein of the present invention, can also be used as a ligand. This compound or salt thereof, which alters the binding properties of a protein showing affinity for a sugar chain with the receptor protein of the present invention, can be obtained by applying the screening method of the present invention using the protein showing an affinity for a sugar chain for example as the ligand.

The compound or salt thereof, which alters the binding properties of a protein showing affinity for a sugar chain with the receptor protein of the present invention, should preferably be a low molecular weight synthetic compound and may be either a novel or a well-known compound. In particular, when screening antagonists to the receptor protein of the present invention, it is desirable to use low molecular weight synthetic compounds having agonist activity in place of proteins showing affinity for sugar chains.

Low molecular weight synthetic compounds are suitable for screening purposes because they are easier to label than proteins showing affinity for sugar chains. These proteins showing affinity for sugar chains and low molecular weight synthetic compounds are referred to collectively below as ligands.

Compounds which alter the binding properties of a ligand with the receptor protein of the present invention include (a) compounds having activity which promotes or activity which suppresses cell stimulus activity or the like via the receptor (so-called agonists to the receptor protein of the present invention), (b) compounds having no such cell stimulus activity (so-called antagonists to the receptor protein of the present invention), (c) compounds which strengthen the binding force of a ligand with the G protein-coupled receptor protein of the present invention, or (d) compounds which weaken the binding force of a ligand with the receptor protein of the present invention or the like (the compounds of (a) above should preferably be screened by the aforementioned ligand determination method).

Examples of cell stimulus activity include for example (1) arachidonic acid release, (2) acetylcholine release, (3) intercellular Ca²⁺ release, (4) intercellular cAMP production, (5) intercellular cGMP production, (6) inositolphosphoric acid production, (7) cell membrane potential fluctuation, (8) phosphorylation of intercellular proteins (such as MAP kinase), (9) activation of c-fos, (10) reduction of pH, (11) activation of Rho, Rac, Ras and other low molecular weight G proteins, and (12) activation of reporter genes (such as luciferase) attached downstream from transcription factor CRE (cAMP responsive element), AP1, NFAT, SRE (serum responsive element) or the like, with promotion of intercellular cAMP production and the like being especially desirable.

That is, the present invention provides a method of screening compounds or salts thereof which alter the binding properties of a ligand with the receptor protein or partial peptide or salt thereof of the present invention, characterized by a comparison of (i) bringing the receptor protein or partial peptide or salt thereof of the present invention into contact with a ligand and (ii) bringing the receptor protein or partial peptide or salt thereof of the present invention into contact with a ligand and a test compound.

The screening method of the present invention is characterized in that for example the amount of binding of the ligand to the receptor protein or the like, cell stimulus activity and the like in cases (i) and (ii) are measured and compared.

More specifically, the present invention provides:

(i) A method of screening compounds or salts thereof which alter the binding properties of a ligand with the receptor protein or the like of the present invention, characterized in that the amount of binding of a labeled ligand with the receptor protein or the like is measured and compared when the labeled ligand is brought into contact with the receptor protein of the present invention and when the labeled ligand and a test compound are brought into contact with the receptor protein of the present invention;

(ii) A method of screening compounds or salts thereof which alter the binding properties of a ligand with the receptor protein or the like of the present invention, characterized in that the amount of binding of a labeled ligand with cells or a membrane fraction is measured and compared when the labeled ligand is brought into contact with cells or a membrane fraction of such cells containing the receptor protein of the present invention or the like, and when the labeled ligand and a test compound are brought into contact with cells or a membrane fraction of such cells containing the receptor protein of the present invention or the like;

(iii) A method of screening compounds or salts thereof which alter the binding properties of a ligand with the receptor protein or the like of the present invention, characterized in that the amount of binding of a labeled ligand with the receptor protein is measured and compared when the labeled ligand is brought into contact with the receptor protein or the like expressed on cell membranes by culturing of a transformant containing the DNA of the present invention, and when the labeled ligand and a test compound are brought into contact with the receptor protein expressed on cell membranes by culturing of a transformant containing the DNA of the present invention;

(iv) A method of screening compounds or salts thereof which alter the binding properties of a ligand with the receptor protein or the like of the present invention, characterized in that cell stimulus activity via a receptor is measured and compared when a compound (such as a ligand) which activates the receptor protein or the like of the present invention is brought into contact with cells containing the receptor protein or the like of the present invention, and when a compound which activates the receptor protein or the like of the present invention and a test compound are brought into contact with cells containing the receptor protein or the like of the present invention; and

(v) A method of screening compounds or salts thereof which alter the binding properties of a ligand with the receptor protein or the like of the present invention, characterized in that cell stimulus activity via a receptor is measured and compared when a compound (such as a ligand) which activates the receptor protein or the like of the present invention is brought into contact with the receptor protein or the like of the present invention expressed on cell membranes by culturing of a transformant containing the DNA of the present invention, and when a compound which activates the receptor protein or the like of the present invention and a test compound are brought into contact with the receptor protein or the like of the present invention expressed on cell membranes by culturing of a transformant containing the DNA of the present invention.

The screening method of the present invention is explained in detail below.

First, the receptor protein or the like of the present invention used in the screening method of the present invention may be any that contains the aforementioned receptor protein or the like of the present invention, but preferably it should be a cell membrane fraction of a mammalian organ containing the receptor protein or the like of the present invention. However, due to the extreme difficulty of obtaining human-derived organs, a human-derived receptor protein or the like made to be expressed in large quantities by means of a recombinant is suited for screening use.

The aforementioned methods can be used to manufacture the receptor protein of the present invention, but it is preferable that the DNA of the present invention be expressed in mammalian cells or insect cells. Complement DNA can be used for a DNA fragment encoding the target protein part, but this is not necessarily a constraint. For example, a gene fragment or synthetic DNA may also be used. For purposes of introducing a DNA fragment encoding the receptor protein of the present invention into host animal cells and causing them to be expressed efficiently, it is preferable that the DNA fragment be incorporated downstream from the polyhedrin promoter of the nuclear polyhedrosis virus (NPV) which belongs to the Baculovirus family having insect hosts, or from a SV40-derived promoter, retrovirus promoter, metallothionein promoter, human heat shock promoter, cytomegalovirus promoter, SR alpha promoter or the like. The amount and quality of expressed receptor can themselves be tested by well-known methods. For example, the methods described in the references (Nambi, P. et al, J. Biol. Chem. 267, 19555-19559, 1992) can be followed.

In the screening method of the present invention, the receptor protein or the like of the present invention may be contained by the receptor protein or the like purified by well-known methods, or cells containing the receptor protein or the like can also be used, as can a cell membrane fraction containing the receptor protein or the like.

When cells containing the receptor protein or the like of the present invention are used in the screening method of the present invention, those cells can be fixed with glutaraldehyde, formalin or the like. Fixing can be according to well-known methods.

The cells containing the receptor protein or the like of the present invention are host cells which express the receptor protein or the like, and E. coli, B. subtilis, yeast, insect or animal cells or the like are preferred as such host cells.

The cell membrane fraction is a fraction containing many cell membranes obtained by well-known methods after crushing of the cells. Examples of cell crushing methods include pounding of cells with a Potter-Elvehjem homogenizer, crushing with a Waring blender or Polytron (Kinematica), crushing by ultrasound or crushing by extruding the cells through a fine nozzle while applying pressure with a French press or the like. A fractioning method using centrifugal force, such as fraction centrifugation or density gradient centrifugation, is generally used for the cell membrane fraction. For example, crushed cell liquid is centrifuged for a short period of time (normally about 1 to 10 minutes) at a low speed (500 rpm to 3000 rpm), the supernatant is then centrifuged for 30 minutes to 2 hours at a high speed (15000 rpm to 30000 rpm), and the resulting precipitate is the membrane fraction. The expressed receptor protein or the like and membrane components such as cell-derived phospholipids and membrane proteins are abundantly present in the membrane fraction.

The amount of receptor protein in the cells or membrane fraction containing the receptor protein or the like is preferably 10³ to 10⁸ molecules per cell and more preferably 10⁵ to 10⁷ molecules per cell. The higher the expressed amount the greater the binding activity (relative activity) of the ligand with respect to the membrane fraction, not only allowing for construction of a highly sensitive screening system, but also for measurement of large volume samples in the same lot.

In order to achieve (1) to (3) above in which compounds are screened which alters the binding properties of a ligand with the receptor protein or the like of the present invention, an appropriate receptor protein fraction and a labeled ligand for example are required.

The receptor protein fraction is desirably either a natural receptor protein fraction or a recombinant receptor protein fraction having activity equivalent thereto or the like. Equivalent activity here signifies equivalent ligand binding activity, signal transmission action or the like.

A labeled ligand, labeled ligand analogue compound or the like can be used as the labeled ligand. For example, a ligand labeled with [³H], [¹²⁵I, [¹⁴C], [³⁵S] or the like can be used.

Specifically, to screen for compounds which alter the binding properties of a ligand with the receptor protein or the like of the present invention, a receptor protein preparation is first prepared by suspending cells or a membrane fraction of cells containing the receptor protein or the like of the present invention in a buffer suitable for screening. The buffer can be any buffer, which does not inhibit binding of the ligand with the receptor protein, such as a Tris-HCl buffer or phosphate buffer of pH 4 to 10 (preferably pH 6 to 8). A surfactant such as CHAPS, Tween-80™ (Kao-Atlas), digitonin, deoxycholate or the like can be added to the buffer for the purpose of reducing non-specific binding. A protease inhibitor such as PMSF, leupeptin E-64 (Peptide Institute), pepstatin or the like can also be added for the purpose of suppressing degradation of the receptor or ligand by proteases. A given amount (5000 cpm to 500000 cpm) of the labeled ligand is added to 0.01 ml to 10 ml of the receptor solution, and 10⁻⁴ M to 10⁻¹⁰ M of the test compound is included at the same time. A reaction tube filled with a large excess of unlabeled ligand is also prepared for investigating non-specific binding (NSB). The reaction is performed for about 20 minutes to 24 hours or preferably about 30 minutes to 3 hours at about 0° C. to 50° C. or preferably at about 4° C. to 37° C. Following the reaction filtration is performed with fiberglass filter paper or the like, and after washing with a suitable amount of the same buffer radioactivity remaining in the fiberglass filter paper is measured with a liquid scintillation counter or gamma counter. If non-specific binding (NSB) is subtracted from the count with no antagonistic substance (B₀) to obtain a count (B₀−NSB) which is given as 10%, a test compound having specific binding (B−NSB) of 50% or less for example can be selected as a candidate substance with competitive inhibition properties.

In order to perform the aforementioned methods of (4) and (5) above for screening compounds which alter the binding properties of a ligand with the receptor protein or the like of the present invention, cell stimulus activity via the aforementioned receptor protein for example can be measured using a well-known method or commercial measurement kit.

Specifically, cells containing the receptor protein or the like of the present invention are first cultured on multi-well plates or the like. For screening purposes fresh medium or a suitable buffer with no toxicity towards the cells is substituted, and the cells are incubated for a fixed time after addition of a test compound or the like, after which the cells are extracted or supernatant collected, and the resulting products are assayed according to the various methods. If production of a substance (for example, arachidonic acid or the like), which is a marker for cell stimulus activity, is difficult to assess due to the presence of a degrading enzyme in the cells, an inhibitor for the degrading enzyme can be added for purposes of the assay. Activity such as cAMP production inhibition or the like can be detected as production inhibition action with respect to cells in which basic production is increased with forskolin or the like.

Cells, which express a suitable receptor protein, are required for screening by measurement of cell stimulus activity. Cell lines having the natural receptor protein or the like of the present invention, or cell lines expressing the aforementioned recombinant receptor protein or the like are desirable as cells which express the receptor protein or the like of the present invention.

A peptide, protein, non-peptide compound, synthetic compound, fermentation product, cell extract, plant extract, animal tissue extract or the like can be used as the test compound, and this compounds may be either a novel compound or a well-known compound.

Compounds designed to bind to the ligand binding pocket based on the atomic coordinates of the active sites of the receptor protein of the present invention and the position of the ligand binding pocket are preferred as test compounds. The atomic coordinates of the active sites of the receptor protein of the present invention and the position of the ligand binding pocket can be measured by well-known methods or methods conforming thereto.

A screening kit for compounds or salts thereof which alter the binding properties of a ligand with the receptor protein or the like of the present invention is one which contains the receptor protein or the like of the present invention, cells containing the receptor protein or the like of the present invention, or a membrane fraction of cells containing the receptor protein or the like of the present invention.

The following are some examples of the screening kit of the present invention.

1. Screening reagents

(1) Measurement Buffer and Washing Buffer

Hanks' Balanced Salt Solution (Gibco) with 0.05% bovine serum albumin (Sigma) added.

Filtered and sterilized with a 0.45 μm filter, stored at 4° C. or may be prepared as needed.

(2) G Protein-Coupled Receptor Preparation

CHO cells made to express the receptor protein of the present invention, passaged 5×10⁵/well on 12-well plates, and incubated for two days at 37° C. in 5% CO₂, 95% air.

(3) Labeled Ligand

Ligand labeled with commercial [³H], [¹²⁵I], [¹⁴C], [³⁵S] or the like

Stored as an aqueous solution at 4° C. or −20° C., diluted for use to 1 μM in measurement buffer.

(4) Ligand Standard Solution

Ligand dissolved to 1 mM in PBS containing 0.1% bovine serum albumin (Sigma), stored at −20° C.

2. Measurement Methods

(1) CHO cells expressing the receptor protein of the present invention, which have been cultured in 12-well tissue culture plates, are washed twice in 1 ml measurement buffer, and 490 μl of measurement buffer is added to each well.

(2) After addition of 5 μl of a 10⁻³ to 10¹⁰ M test compound solution, 5 PI of labeled ligand is added and reacted for 1 hour at room temperature. 5 μl of 10⁻³ M ligand is added in place of the test compound to investigate non-specific binding.

(3) The reaction liquid is removed, and washing performed 3 times with 1 ml of washing buffer. Labeled ligand bound to the cells is dissolved with 0.2 N NaOH-1% SDS, and mixed with 4 ml of liquid scintillator A (Wako Pure Chemical).

(4) Radioactivity is measured using a liquid scintillation counter (Beckman), and percent maximum binding (PMB) is derived from the following formula:
PMB=[(B−NSB)/(B₀−NSB)]×100
PMB: percent maximum binding
B: Value when specimen added
NSB: Non-specific binding
B₀ Maximum binding

Either (i) or (ii) below can be followed as the specific method of evaluating whether a compound or salt thereof which alters the binding properties of a ligand with a receptor protein or the like of the present invention is an agonist or antagonist.

(i) After the binding assay shown in the screening method of 1-3 above has been performed to obtain a compound which alters (or particularly which inhibits) the binding properties of a ligand with a receptor protein or the like of the present invention, measurements are performed to determine whether the compound has cell stimulus activity via the aforementioned receptor protein of the present invention. Compounds or salts thereof, which have cell stimulus activity, are agonists, while compounds or salts thereof having no such activity are antagonists.

(ii) (a) The test compound is brought into contact with cells containing the receptor protein of the present invention, and cell stimulus activity via the receptor protein of the present invention is measured. Compounds or their salts having cell stimulus activity are agonists.
(b) Cell stimulus activity via the receptor protein of the present invention is measured and compared when a compound (for example, a ligand, agonist to the receptor protein of the present invention or the like) which activates the receptor protein of the present invention is brought into contact with cells containing the receptor protein of the present invention, and when a compound which activates the receptor protein of the present invention and a test compound are brought into contact with cells containing the receptor protein of the present invention. Compounds or salts thereof, which can reduce cell stimulus activity due to compounds that activate the receptor protein of the present invention, are antagonists.

Compounds or salts thereof obtained using the screening method or screening kit of the present invention are compounds which act to alter the binding properties of a ligand with the receptor protein or the like of the present invention, and specifically they are (1) compounds having cell stimulus activity via the aforementioned receptor protein of the present invention (so called agonists to the receptor protein of the present invention), (2) compounds having no such cell stimulus activity (so-called antagonists to the receptor protein of the present invention), (3) compounds which increase the binding force of the ligand with the receptor protein of the present invention and (4) compounds which reduce the binding force of the ligand with the receptor protein of the present invention.

Examples of such compounds include peptides, proteins, non-peptide compounds, synthetic compounds, fermentation products and the like, and these compounds may be either novel compounds or well-known compounds.

Since agonists to the receptor protein or the like of the present invention have action equivalent to the physiological activity of the aforementioned ligand with respect to the receptor protein or the like of the present invention, they are useful as safe, low-toxicity drugs according to the physiological activity.

Since agonists to the receptor protein or the like of the present invention are capable of suppressing the physiological activity of the aforementioned ligand with respect to the receptor protein or the like of the present invention, they are useful as safe, low-toxicity drugs for suppressing such physiological activity.

Compounds, which increase the binding force of the ligand with the receptor protein of the present invention, are useful as safe, low-toxic drugs because they augment the physiological activity of the ligand with respect to the receptor protein of the present invention.

Compounds, which decrease the binding force of the ligand with the receptor protein of the present invention, are useful as safe, low-toxicity drugs because they reduce the physiological activity of the ligand with respect to the receptor protein of the present invention.

Specifically, compounds or salts thereof obtained by the screening method or screening kit of the present invention which act to alter the binding properties of a ligand with a receptor protein of the present invention are useful as birth inducers and as preventative and therapeutic drugs for disorders such as prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency, calculus and the like.

(7) Preventative and/or Therapeutic Agent for Various Disorders Containing a Compound (Agonist, Antagonist or the Like) which Alters the Binding Properties of the Receptor Protein of the Present Invention with a Ligand

A compound (agonist, antagonist) which alters the binding properties of the protein of the present invention with a ligand or the aforementioned ligand for the receptor protein of the present invention can be used as a birth inducer or as a preventative and/or therapeutic agent for disorders associated with dysfunction of the receptor protein of the present invention, such as prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency and calculus and the like.

When such a compound or ligand is used as a preventative and/or therapeutic agent for a disorder associated with dysfunction of the receptor protein of the present invention, it can be formulated according to the ordinary methods described above.

(8) Assay of Proteins and the like using Antibodies of the Present invention, Diagnostic Drug Containing Antibodies of the Present Invention and Diagnostic Method Employing Same

Since the antibodies of the present invention can specifically recognize the receptor protein or the like of the present invention, they can be used in assaying the receptor protein or the like of the present invention in a test liquid, and particular in assaying by sandwich immunoassay, competitive methods, immunometric methods, nephrometry and the like.

Special conditions or operations do not need to be devised for applying any of these immunoassay methods to the measurement method of the present invention. It is sufficient to construct a measurement system for the receptor protein or salt thereof of the present invention with the ordinary technical considerations of a person having ordinary skill in the field added to the normal conditions and operations for the respective methods. General texts and manuals can be consulted with regard to the details of these ordinary technical methods [see for example, Irie, Hiroshi ed. Radioimmunoassay (Kodansha, 1974), Irie, Hiroshi ed. Radioimmunoassay Continued (Kodansha, 1979), Ishikawa, Eiji et al ed. Enzyme Immunoassay (Igaku Shoin, 1978), Ishikawa, Eiji et al ed. Enzyme Immunoassay 2^{nd} edition (Igaku Shoin, 1982), Ishikawa, Eiji et al ed. Enzyme Immunoassay 3^{rd} edition (Igaku Shoin, 1987), Methods in Enzymology Vol. 70 Immunochemical Techniques (Part A), Vol. 73 Immunochemical Techniques (Part B), Vol. 74 Immunochemical Techniques (Part C), Vol. 84 Immunochemical Techniques (Part D: Selected Immunoassays), Vol. 92 Immunochemical Techniques (Part E: Monoclonal Antibodies and General Immunoassay Methods), and Vol. 121 Immunochemical Techniques (Part I: Hybridoma Technology and Monoclonal Antibodies) (all Academic Press), WO publication 00/14227 page 39, line 25 through page 42, line 8, EP publication #1111047A2 paragraph [0115] page 19, line 35 through page 20, line 47].

As described above, the antibodies of the present invention can be used to sensitively assay the receptor protein or salt thereof of the present invention.

Moreover, it is also possible to diagnose various diseases associated with dysfunction of the receptor protein of the present invention by using the antibodies of the present invention to assay the receptor protein or salt thereof of the present invention in vivo.

For example, if an increase or decrease in the concentration of the receptor protein is detected in an assay of the concentration of the receptor protein of the present invention using the antibodies of the present invention, it is possible to diagnose a high likelihood of dysfunction or over-expression of the receptor protein for example, particularly prostate cancer, other cancers (non-small cell carcinoma, ovarian cancer, stomach cancer, bladder cancer, breast cancer, cervical cancer, colon cancer, rectal cancer large intestinal cancer), prostatomegaly, male gonad dysfunction, infertility, premature birth, endometriosis, cirrhosis of the liver, hepatitis, hepatic insufficiency or calculus, or a high likelihood of such dysfunction occurring in the future.

The antibodies of the present invention can also be used to specifically detect the receptor protein or the like of the present invention, which is present in a test specimen such as bodily fluid or tissue. They can also be used for preparing an antibody column to be used for purifying the receptor protein or the like of the present invention, for detecting the receptor protein or the like of the present invention in the various fractions during purification, and for analyzing the behavior of the receptor protein of the present invention in test cells.

(9) Method of Screening Compounds that Alter the Amount of the Receptor Protein or Partial Peptide thereof in Cell Membranes

Since the antibodies of the present invention can specifically recognize the receptor protein or partial peptide or salt thereof of the present invention, they can be used to screen compounds, which alter the amount of the receptor protein or partial peptide thereof in cell membranes.

Namely, the present invention provides the following screening methods for example:

(i) A method of screening compounds which alter the amount of the receptor protein or partial peptide thereof of the present invention in cell membranes, wherein a) blood, b) specific organs, or c) tissue, cells or the like isolated from specific organs of non-human mammals are ground, the cell membrane fraction is isolated, and the receptor protein or partial peptide thereof of the present invention contained in the cell membrane fraction is assayed;

(ii) A method of screening compounds which alter the amount of the receptor protein or partial peptide thereof of the present invention in cell membranes, wherein a transformant or the like which expresses the receptor protein or partial peptide thereof of the present invention is crushed, the cell membrane fraction is isolated, and the receptor protein or partial peptide thereof of the present invention contained in the cell membrane fraction is assayed;

(iii) A method of screening compounds which alter the amount of the receptor protein or partial peptide thereof of the present invention in cell membranes, wherein a) blood, b) specific organs, or c) tissue, cells or the like isolated from specific organs of non-human mammals are sliced, and the degree of staining of the receptor protein in the cell cortex is quantified by immune staining in order to confirm the protein on the cell membranes; and

(iv) A method of screening compounds which alter the amount of the receptor protein or partial peptide thereof of the present invention in cell membranes, wherein a transformant or the like which expresses the receptor protein or partial peptide thereof of the present invention is sliced, and the degree of staining of the receptor protein in the cell cortex is quantified by immune staining in order to confirm the protein on the cell membranes.

Specifically, the receptor protein or partial peptide thereof of the present invention in a cell membrane fraction is assayed as follows.

(i) A drug (such as an anti-cancer drug) or physical stress (such as immersion stress, electric shock, brightness contrast or low temperature) or the like is applied to normal or disease-model non-human mammals (such as mice, rats, rabbits, sheep, pigs, cows, cats, dogs, monkeys or the like, or more specifically cancer-carrying mice), and after a fixed amount of time blood or specific organs (such as brains, lungs, large intestines, prostate glands or the like) or tissue or cells isolated from organs are obtained. The resulting organs, tissues, cells or the like are suspended for example in suitable buffer (such as a tris-hydrochloric acid buffer, phosphoric acid buffer or Hepes buffer) or the like, the organs, tissues or cells are crushed, and a cell membrane fraction is obtained by a method such as centrifugation, filtration, column fractioning or the like using a surfactant (such as Triton x 100™ or Tween 20™) or the like.

The cell membrane fraction is a fraction containing many cell membranes, which is obtained by well-known methods after crushing of the cells. Examples of cell crushing methods include pounding of cells with a Potter-Elvehjem homogenizer, crushing with a Waring blender or Polytron (Kinematica), crushing by ultrasound or crushing by extruding the cells through a fine nozzle while applying pressure with a french press or the like. A fractioning method using centrifugal force, such as fraction centrifugation or density gradient centrifugation, is generally used for the cell membrane fraction. For example, crushed cell liquid is centrifuged for a short period of time (normally about 1 to 10 minutes) at a low speed (500 rpm to 3000 rpm), the supernatant is then centrifuged for 30 minutes to 2 hours at a high speed (15000 rpm to 30000 rpm), and the resulting precipitate is the membrane fraction. The expressed receptor protein and the like and membrane components such as cell-derived phospholipids and membrane proteins are abundantly present in the membrane fraction.

The receptor protein or partial peptide thereof of the present invention contained in the cell membrane fraction can be assayed for example by Western blotting analysis or sandwich immunoassay using the antibodies of the present invention or the like.

Sandwich immunoassay can be performed by the same methods as those described above, while Western blotting can be performed by well-known methods.

(ii) A transformant expressing the receptor protein or partial peptide thereof of the present invention can be prepared by the methods described above, and the receptor protein or partial peptide thereof of the present invention contained in the cell membrane fraction assayed.

Compounds, which alter the amount of the receptor protein or partial peptide thereof of the present invention in cell membranes can be screened by:

(i) Administering a test compound to normal or disease model non-human mammals a fixed time before (30 minutes to 24 hours before, preferably 30 minutes to 12 hours before, more preferably 1 hour to 6 hours before) or a fixed time after (30 minutes to 3 days after, preferably 1 hour to 2 days after, more preferably 1 hour to 24 hours after) application of a drug, physical stress or the like or simultaneously with the drug or physical stress, and assaying and analyzing the amount of the receptor protein or partial peptide thereof of the present invention in cell membranes a fixed time after administration (30 minutes to 3 days, preferably 1 hour to 2 days, more preferably 1 hour to 24 hours); or

(ii) Mixing a test compound into the medium when culturing the transformant by ordinary methods, and assaying the amount of the receptor protein or partial peptide thereof of the present invention cell membranes after a fixed culture time (1 day to 7 days, preferably 1 day to 3 days, more preferably 2 days to 3 days).

Specifically, the receptor protein or partial peptide thereof of the present invention contained in a cell membrane fraction can be confirmed as follows.

(iii) A drug (such as an anti-dementia drug, blood pressure lowering drug, anti-cancer drug, anti-obesity drug or the like) or physical stress (such as immersion stress, electric shock, brightness contrast or low temperature) or the like is applied to normal or disease-model non-human mammals (such as mice, rats, rabbits, sheep, pigs, cows, cats, dogs, monkeys or the like, or more specifically dementia rats, obese mice, arteriosclerotic rabbits, cancer-carrying mice or the like), and after a fixed amount of time blood or specific organs (such as brains, lungs, large intestines or the like) or tissue or cells isolated from organs are obtained. The resulting organs, tissues, cells or the like are tissue sliced by conventional methods, and immune stained with the antibodies of the present invention. By quantifying the amount of staining of the receptor protein on the cell cortex and confirming the protein on the cell membranes, it is possible to confirm the amount of the receptor protein or partial peptide thereof of the present invention in cell membranes either quantitatively or qualitatively.

(iv) The same can be confirmed by the same methods using a transformant or the like which expresses the receptor protein or partial peptide thereof of the present invention.

A compound or salt thereof obtained by the screening method of the present invention is a compound having the action of altering the amount of the receptor protein or partial peptide thereof of the present invention in cell membranes, and specifically it is (a) a compound which augments cell stimulus activity via the receptor by increasing the amount of the receptor protein or partial peptide thereof of the present invention in cell membranes, or (b) a compound which weakens cell stimulus activity by decreasing the amount of the receptor protein or partial peptide thereof of the present invention in cell membranes.

Examples of this compound include peptides, proteins, non-peptide compounds, synthetic compounds, fermentation products and the like, and these compounds may be either novel compounds or well-known compounds.

A compound, which augments cell stimulus activity is useful as a safe, low-toxic drug for augmenting the physiological activity of the receptor protein or the like of the present invention.

A compound, which weakens cell stimulus activity is useful as a safe, low-toxic drug for decreasing the physiological activity of the receptor protein or the like of the present invention.

(10) Compounds, which Alter the Amount of the Receptor Protein or Partial Peptide thereof of the Present Invention in Cell Membranes, and Preventative and/or Therapeutic Agents Containing such Compounds

As described above, the receptor protein of the present invention is thought to play an important role in vivo for example. Consequently, a compound, which alters the amount of the receptor protein or partial peptide thereof of the present invention in cell membranes, can be used as a preventative and/or therapeutic agent for disorders associated with dysfunction of the receptor protein of the present invention. For example, such a compound can be used as a birth inducer or as a preventative and/or therapeutic drug for disorders caused by over-expression and the like of this receptor protein (such as prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency, calculus and other disorders).

When this compound is used as a preventative and/or therapeutic agent for a disorder associated with dysfunction of the receptor protein of the present invention, it can be formulated according to the ordinary methods described above.

(11) Drug Containing Antibodies to the Receptor Protein or Partial Peptide or Salt thereof of the Present Invention

The neutralizing activity of antibodies to the receptor protein or partial peptide or salt thereof of the present invention with respect to the receptor protein and the like is activity which inactivates activity associated with the receptor protein, such as signal transmission. Consequently, when such antibodies have neutralizing activity, they can inactivate signal transmission associated with the receptor protein, including cell stimulus activity via the receptor protein (such as activity which promotes or suppresses arachidonic acid release, acetylcholine release, intercellular Ca²⁺ release, intercellular cAMP production, intercellular cAMP suppression, intercellular cGMP production, inositolphosphoric acid production, cell membrane potential fluctuation, phosphorylation of intercellular proteins, activation of c-fos, reduction of pH or the like, particularly promotion activity of intercellular cAMP production). Consequently, such antibodies can be used in the prevention and/or treatment of disorders caused by over-expression and the like of this receptor protein. For example, neutralizing antibodies to the receptor protein of the present invention can be used as a birth inducer or as a preventative and/or therapeutic drug for disorders stemming from over-expression of this receptor protein (such as prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency, calculus and other disorders).

The formulation, dosage, administration method and the like described above for the drug of the present invention can be used (see “Means of diagnosing diseases and drugs for treating those diseases”).

(12) Preparation of Animals with Introduced DNA of the Present Invention

The present invention provides non-human mammals having extrinsic DNA of the present invention (abbreviated hereunder as extrinsic DNA of the present invention) or mutant DNA thereof (sometimes abbreviated hereunder as extrinsic mutant DNA of the present invention).

Namely, the present invention provides:

[1] Non-human mammals having extrinsic DNA of the present invention or mutant DNA thereof;

[2] The animals according to [1], wherein the non-human mammals are rodents;

[3] The animals according to [2], wherein the rodents are mice or rats; and

[4] A recombinant vector containing extrinsic DNA of the present invention or mutant DNA thereof, and capable of expression in mammals.

Non-human mammals having extrinsic DNA of the present invention or mutant DNA thereof (abbreviated hereunder as DNA transposed animals of the present invention) can be created by transposing the target DNA into germinal cells or the like including unfertilized eggs, fertilized eggs, sperm and initial cells thereof, preferably at the embryogenesis stage of non-human mammal development (and more preferably at the single-cell or fertilized egg cell stage, and generally before the 8-cell stage) by the calcium phosphate method, electrical pulse method, lipofection method, agglutination method, micro-injection method, particle gun method, DEAE-dextran method or the like. These DNA transposition methods can also be used to transpose target extrinsic DNA of the present invention into somatic cells, living organs, tissue cells or the like, which are then used for cell cultures or tissue cultures, or animals having the introduced DNA of the present invention can be created by fusing these cells with the aforementioned germinal cells by well-known methods of cell fusion.

Cows, pigs, sheep, goats, rabbits, dogs, cats, guinea pigs, hamsters, mice, rats or the like for example can be used as the non-human mammals. Of these, rodents and particularly mice (such as pure strains C57 BL/6, DBA2 or the like and hybrid strains B6C3F₁, BDF₁, B6D2F₁, BALB/c, ICR and the like) and rats (such as Wistar, SD or the like) are desirable for creating animal disease models because their ontogenesis and life cycles are relatively short and because they are easy to breed.

In addition to the aforementioned non-human mammals, humans and the like are also possible as the “mammals” for the recombinant vector capable of expression in mammals.

The extrinsic DNA of the present invention is not DNA of the present invention originally present in non-human mammals, but DNA of the present invention which has been isolated or extracted from mammals.

DNA or the like wherein a modification (such as a mutation) has occurred in the nucleotide sequence of the original DNA of the present invention, specifically DNA wherein a base addition, deletion or replacement by another base has occurred, can be used as the mutant DNA of the present invention, and abnormal DNA is also included.

Such abnormal DNA signifies DNA which expresses the abnormal receptor protein of the present invention, and for example DNA and the like can be used which expresses a receptor protein which suppresses the function of the normal receptor protein of the present invention.

The extrinsic DNA of the present invention may be derived from either the same species or a different species of mammal than the target animal. For introducing the DNA of the present invention into the target animal, it is normally useful to use a DNA construct having the DNA bound downstream from a promoter capable of promoting expression in animal cells. For example, when introducing human DNA of the present invention, a DNA construct (such as a vector) having the human DNA of the present invention bound downstream from various promoters capable of promoting expression of DNA derived from various mammals (such as rabbits, dogs, cats, guinea pigs, hamsters, rats, mice and the like) having DNA of the present invention with high homology to the human DNA can be microinjected into the fertilized eggs of target mammals (such as fertilized mouse eggs) to create a transgenic mammal which highly expresses the DNA of the present invention.

An E. coli-derived plasmid, B. subtilis-derived plasmid, yeast-derived plasmid, gamma phage or other bacteriophage, Moloney leukemia virus or other retrovirus or Vaccinia virus or other baculovirus or another animal viruscan be used as the expression vector for the receptor protein of the present invention. Of these, an E. coli-derived plasmid, B. subtilis-derived plasmid or yeast-derived plasmid is used by preference.

As the promoter which regulates the aforementioned DNA expression, it is possible to use (1) a DNA promoter derived from a virus (such as a simian virus, cytomegalovirus, Moloney leukemia virus, JC virus, breast cancer virus, polio virus or the like) or (2) a promoter derived from various mammals (such as humans, rabbits, dogs, cats, guinea pigs, hamsters, rats, mice and the like), such as an albumin, insulin II, uroplakin II, elastase, erithropoietin, endothelin, muscle creatine kinase, glial fibrillary acidic protein, glutathione S transferase, platelet-derived growth factor beta, keratin K1, K10 and K14, collagen I and II, cyclic AMP-dependent protein kinase beta I subunit, dystrophin, tartrate-resistant alkali phosphatase, atrial natriuretic factor, endothelial receptor tyrosine kinase (generally abbreviated as Tie2), Na,K-ATPase, neurofilament light chain, metallothionein I and IIA, metalloproteinase I tissue inhibitor, MHC class I antibody (H-2L), H-ras, renin, dopamine beta hydroxylase, thyroid peroxidase (TPO), peptide chain elongation factor 1α (EF-1α), beta actin, alpha and beta myosin heavy chains, myosin light chains 1 and 2, myelin basic protein, thyroglobulin, Thy-1, immunoglobulin, H chain variable region (VNP), serum amyloid P component, myoglobin, troponin C, smooth muscle alpha actin, preproenkephalin A, vasopressin and other promoters. Of these, the cytomegalovirus promoter, human peptide chain elongation factor 1α (EF-1α) promoter, human and chicken beta actin promoters and the like which are capable of high expression throughout the body are preferred.

The aforementioned vector preferably has a sequence (normally called a terminator) which terminates transcription of the target mRNA in the mammal having introduced DNA. For example, the respective DNA sequences derived from viruses or various mammals can be used, and the simian virus SV40 terminator is used by preference.

It is also possible depending on the objective to attach the respective DNA splicing signals, enhancer regions, part of eukaryotic DNA introns or the like on the 5′ upstream side of the promoter region, between the promoter region and the translation region or on the 3′ downstream side of the translation region in order to induce even higher expression of the extrinsic DNA of the present invention.

The translation region of the normal receptor protein of the present invention may be obtained as DNA derived from livers, kidneys, thyroid glands or fibroblasts from humans and various other mammals (such as rabbits, dogs, cats, guinea pigs, hamsters, rats, mice and the like) and as all or part of genomic DNA from various commercial genome DNA libraries, or else complement DNA prepared by well-known methods from RNA derived from livers, kidneys, thyroid glands or fibroblasts may be obtained as raw material. In the case of extrinsic abnormal DNA, the translation region of the normal receptor protein of the present invention obtained from the aforementioned cells or tissues can be mutated by point mutagenesis to prepare a mutant translation region.

This translation region can be prepared as a DNA construct capable of expression in transgenic animals by ordinary DNA engineering methods of ligation downstream from the promoter and if desired upstream from the transcription termination site.

Introduction of the extrinsic DNA of the present invention at the fertilized egg cell stage ensures that it will be present in all germinal cells and somatic cells of the target mammal. Having extrinsic DNA of the present invention present in germinal cells of the resulting animal after DNA introduction means that all the progeny of the resulting animal will have the extrinsic DNA of the present invention in all their germinal and somatic cells. The descendents of animals of this type which inherit the extrinsic DNA of the present invention will have extrinsic DNA of the present invention in all their germinal and somatic cells.

Non-human mammals having introduced extrinsic normal DNA of the present invention can be bred to confirm the stable presence of the extrinsic DNA, and successive generations can be reared in a normal environment as animals having this DNA.

Introduction of the extrinsic DNA of the present invention at the fertilized egg stage ensures that it is present in excess in all germinal and somatic cells of the target mammals. Having an excess of the extrinsic DNA of the present invention present in germinal cells of the resulting animal after DNA introduction means that all progeny of the resulting animal will have an excess of the extrinsic DNA of the present invention in all their germinal and somatic cells. The descendents of animals of this type which inherit the extrinsic DNA of the present invention will have an excess of the extrinsic DNA of the present invention in all their germinal and somatic cells.

Homozygous animals can be obtained having the introduced DNA in both homologous chromosomes, and male and female animals of this type crossed to breed successive generations in which all progeny have the DNA in excess.

The normal DNA of the present invention is highly expressed in non-human mammals having the normal DNA of the present invention, promoting the function of intrinsic normal DNA and ultimately leading in some cases to hyperfunction of the receptor protein of the present invention, for which they can be used as disease model animals. For example, animals having normal introduced DNA of the present invention can be used to elucidate the pathological mechanisms of hyperfunction of the receptor protein of the present invention and of diseases associated with the receptor protein of the present invention, and to investigate therapy methods for these diseases.

Moreover, since mammals having introduced extrinsic normal DNA of the present invention exhibit symptoms of increased free receptor protein of the present invention, they can also be used in screening tests for therapeutic drugs for diseases associated with the receptor protein of the present invention.

On the other hand, non-human mammals having extrinsic abnormal DNA of the present invention can be bred to confirm the stable presence of the extrinsic DNA, and successive generations reared in a normal environment as animals having this DNA. Moreover, the target extrinsic DNA can be incorporated into the aforementioned plasmid and used as raw material. A DNA construct with a promoter can be prepared by normal DNA engineering techniques. Introduction of the abnormal DNA of the present invention at the fertilized egg stage ensures that it will be present in all germinal and somatic cells of the target mammal. Having abnormal DNA of the present invention present in germinal cells of the resulting animal after DNA transposition means that all progeny of the resulting animal will having the abnormal DNA of the present invention in all their germinal and somatic cells. The descendents of animals of this type which inherit the extrinsic DNA of the present invention will have abnormal DNA of the present invention in all their germinal and somatic cells. Homozygous animals can be obtained having the introduced DNA in both homologous chromosomes, and these male and female animals crossed to breed successive generations in which all the progeny have this DNA.

The abnormal DNA of the present invention is highly expressed in non-human mammals having the abnormal DNA of the present invention, inhibiting the function of intrinsic normal DNA and ultimately leading in some cases to refractory disease with functionally inactive receptor protein of the present invention, for which such animals can be used as disease model animals. For example, animals having abnormal introduced DNA of the present invention can be used to elucidate the pathological mechanisms of refractory disease with functionally inactive receptor protein of the present invention, and to investigate therapy methods for this condition.

One specific potential use for animals highly expressing the abnormal DNA of the present invention is as a model for elucidating the functional inhibition (dominant negative effect) of the normal receptor protein by the abnormal receptor protein of the present invention in cases of refractory disease with functionally inactive receptor protein of the present invention.

Moreover, since mammals having introduced extrinsic abnormal DNA of the present invention exhibit symptoms of increased free receptor protein of the present invention, they can also be used in screening tests for therapeutic drugs for refractory disease with functionally inactive receptor protein of the present invention.

Other potential uses for the two aforementioned types of animals with introduced DNA of the present invention are for example:

(1) As a cell source for tissue cultures:

(2) For analyzing relatedness with the receptor protein of the present invention specifically expressed or activated by the receptor protein of the present invention, either by directly analyzing DNA or RNA in tissues of DNA transposed animals of the present invention or by analyzing the receptor protein of the present invention expressed by the DNA;

(3) For culturing cells of tissues having DNA according to standard tissue culture techniques, and using them to research the functions of cells from tissues which are ordinarily difficult to culture;

(4) For screening drugs which enhance the functions of cells using the cells according to 3 above; and

(5) For isolating and purifying the mutant receptor protein of the present invention and preparing antibodies thereto.

Moreover, animals having introduced DNA of the present invention can be used to investigate clinical symptoms of diseases associated with the receptor protein of the present invention, including refractory disease with functionally inactive receptor protein of the present invention, and more precise pathological findings can be obtained for various organs of a disease model for a disease associated with the receptor protein of the present invention, contributing to the development of novel therapy methods and to research and therapy for secondary disorders stemming from these disorders.

Also, various organs can also be removed from animals having introduced DNA of the present invention and chopped, and trypsin or another protease can be used to obtain free cells with introduced DNA, which can be cultured or the cultured cells established as a strain. Relatedness with the specification, apoptosis, differentiation or proliferation of cells producing the receptor protein of the present invention can also be investigated as well as the signal transmission mechanisms of these, and abnormalities thereof can also be investigated, providing useful research material for studying the receptor protein of the present invention and its action.

Moreover, in order to develop therapeutic drugs for disorders associated with the receptor protein of the present invention, including refractory disease with functionally inactive receptor protein of the present invention, effective and rapid methods of screening therapeutic drugs for such disorders using the aforementioned testing and assay methods and the like can be provided using animals having introduced DNA of the present invention. Animals having introduced DNA of the present invention or extrinsic DNA-expressing vectors of the present invention can also be used to investigate and develop DNA therapies for disorders associated with the receptor protein of the present invention.

(13) Knockout Animals

The present invention provides non-human mammalian embryonic stem cells wherein the DNA of the present invention is inactivated, and non-human mammals having insufficient expression of the DNA of the present invention.

Namely, the present invention provides:

[1) Non-human mammalian embryonic stem cells wherein the DNA of the present invention is inactivated;

[2] The embryonic stem cells according to [1], wherein the DNA is inactivated by introduction of a reporter gene (such as an E. coli-derived beta-galactosidase gene);

[3] The embryonic stem cells according to [1], which are neomycin resistant;

[4] The embryonic stem cells according to [1], wherein the non-human mammal is a rodent;

[5] The embryonic stem cells according to [4], wherein the rodent is a mouse;

[6] A non-human mammal expressing the DNA of the present invention wherein the DNA is inactivated;

[7] The non-human mammal according to [6], wherein the DNA is inactivated by introduction of a reporter gene (such as an E. coli-derived beta-galactosidase gene), and wherein the reporter gene can be expressed under the control of a promoter for the DNA of the present invention;

[8] The non-human mammal according to [6], wherein the non-human mammal is a rodent;

[9] The non-human mammal according to [8], wherein the rodent is a mouse; and

[10] A method of screening compounds or salts thereof which promote or inhibit promoter activity for the DNA of the present invention, wherein a test compound is administered to the animals according to [7], and expression of the reporter gene is detected.

Non-human mammalian embryonic stem cells wherein the DNA of the present invention is inactivated are the embryonic stem cells (abbreviated below as ES cells) of non-human mammals wherein either DNA expression ability is suppressed by the artificial addition of a mutation into DNA of the present invention present in the non-human mammals, or wherein the DNA has substantially no ability to express the receptor protein of the present invention because the activity of the receptor protein of the present invention encoded by this DNA has been substantially eliminated (sometimes referred to hereunder as knockout DNA of the present invention).

The non-human mammals used may be as described above.

Methods of artificially introducing a mutation into the DNA of the present invention include for example deleting all or part of the DNA sequence by genetic engineering techniques, or inserting or substituting other DNA. With such a mutation, knockout DNA of the present invention can be created for example by shifting the codon reading frame or destroying the function of the promoter or exon.

Specific examples of non-human mammalian embryonic stem cells wherein the DNA of the present invention is inactivated (abbreviated hereunder as DNA inactivated ES cells of the present invention or knockout ES cells of the present invention) can be obtained for example by isolating DNA of the present invention present in the target non-human mammals and either destroying the function of the exon by inserting into the exon a neomycin-resistant gene, hygromycin-resistant gene or other drug-resistant gene or a reporter gene such as lacZ (beta-galactosidase gene) or cat (chloramphenicol acetyltransferase gene), or else preventing synthesis of complete mRNA by inserting a DNA sequence (such as a polyA addition signal or the like) which terminates transcription of the gene into an intron region among the exons. A DNA chain (abbreviated hereunder as the targeting vector) having a DNA sequence constructed with the gene destroyed in this way is introduced into chromosomes of the animal by homologous recombination for example, the resulting ES cells are analyzed either by Southern hybridization using a DNA sequence on or near the DNA of the present invention as a probe or by PCR using the DNA sequence of the targeting vector and the DNA sequence of a neighboring region other than the DNA of the present invention used in preparing the targeting vector as the primers, and knockout ES cells of the present invention are selected.

The original ES cells for inactivating the DNA of the present invention by homologous recombination or the like may be already established cells as described above or may be newly established by the well-known methods of Evans and Kaufman. For example, in the case of mouse ES cells, 129 strains of ES cells are currently in general use, but since the immunological backgrounds are unclear, those established using C57BL/6 mice or BDF₁ mice (C57BL/6, DBA/2 and F₁) wherein the low egg yield of C57BL/6 mice is improved by cross-breeding with DBA/2 or the like are used instead by preference in order to obtain pure-strain ES cells with a clear immunological background. BDF₁ mice offer the advantages of high egg yield and sturdy eggs, and since they are based on C57BL/6 mice, ES cells obtained therefrom can be used advantageously because when disease model mice are created they can be back-crossed with C57BL/6 mice in order to replace the genetic background with that of C57BL/6 mice.

Blastocysts 3.5 days after fertilization are generally used for establishing ES cells, but alternatively many early embryos can be efficiently obtained by collecting 8-celled embryos and culturing them into blastocysts.

Either female or male ES cells may be used, but normally male ES cells are convenient for creating a reproductive series chimera. It is also desirable to distinguish female and male as soon as possible in order to reduce the work of a complicated culture.

One method of distinguishing female and male ES cells is for example to amplify and detect the gene of the sex-determining region of the Y chromosome by PCR. Using this method, only about 1 colony of ES cells (about 50 cells) is required in contrast to the approximately 10⁶ cells required by conventional karyotype analysis, making it possible to perform primary selection of ES cells at the initial culture stage by distinguishing female and male, and therefore greatly reducing the work of the initial culture stage by allowing male cells to be selected early on.

Secondary selection can be performed for example by confirmation of number of chromosomes by G-banding or the like. The number of chromosomes in the resulting ES cells is desirably 100% of the normal number, but when there are difficulties involving physical operations and the like during cell establishment, it is desirable that they are re-cloned to normal cells (for example, cells with 2n=40 chromosomes in the case of mice) after knocking out of the ES cell gene.

The resulting embryonic stem cell line is normally highly prolific, but careful subculturing is required because individual developmental potency is easily lost. For example, a method can be adopted such as culturing at about 37° C. in a carbon dioxide incubator (preferably with 5% carbon dioxide, 95% air or 5% oxygen, 5% carbon dioxide, 90% air) with LIF (1 to 10000 U/ml) on suitable feeder cells such as STO fibroblasts, and for subculture treating with trypsin-EDTA solution (normally 0.001 to 0.5% trypsin/0.1 to 5 mM EDTA, preferably about 0.1% trypsin/1 mM EDTA) for example to obtain single cells which are inoculated on newly-prepared feeder cells. Such subcultures are normally performed once every 1 to 3 days, at which time the cells are observed and if cells are found with morphological abnormalities such culture cells should desirably be discarded.

ES cells can be differentiated into various types of cells including parietal muscle, visceral muscle, cardiac muscle and the like by monolayer culturing them to high densities under suitable conditions, or float culturing them until they form a cell aggregate (M. J. Evans and M. H. Kaufman, Nature 292, p. 154, 1981; G. R. Martin, Proc. Natl. Acad. Sci. U.S.A. 78, p. 7634, 1981; T. C. Doetschman et al, Journal of Embryology and Experimental Morphology 87, p. 27, 1985), and cells with insufficient expression of the DNA of the present invention obtained by differentiation of ES cells are useful for the receptor protein of the present invention in vitro or for cytobiological study of the receptor protein of the present invention.

Non-human mammals with insufficient expression of the DNA of the present invention can be distinguished from normal animals by measuring the mRNA amounts of the animals by well-known methods and comparing the expressed amounts indirectly.

The non-human mammals used may be as described above.

The non-human mammals with insufficient expression of the DNA of the present invention can for example have the DNA of the present invention knocked out by introducing a targeting vector prepared as described above into mouse embryonic stem cells or mouse eggs cells, and using homologous gene recombination to replace the DNA of the present invention on the chromosomes of the mouse embryonic stem cells or mouse egg cells with a DNA sequence having the DNA of the present invention of the targeting vector inactivated by introduction.

Cells with knocked-out DNA of the present invention can be determined by Southern hybridization analysis using a DNA sequence on or near the DNA of the present invention as the probe, or by PCR analysis using as the primers a DNA sequence on the targeting vector and the DNA sequence of a neighboring region other than the mouse-derived DNA of the present invention used as the targeting vector. When using non-human mammalian embryonic stem cells, a cell strain having the DNA of the present invention inactivated by homologous gene recombination is cloned, the cells are injected at a suitable cell stage such as into 8-cell non-mammalian embryos or blastocysts, and the resulting chimera embryos are transplanted into the uteri of the non-human mammals which have been made pseudopregnant. The resulting animals are chimera animals consisting of both cells with the normal DNA locus of the present invention and cells with the artificially mutated gene locus of the present invention.

When part of the reproductive cells of these chimera animals have the mutated DNA locus of the present invention, individuals in which all tissues are composed of cells having the DNA locus of the present invention with the artificial mutation can be selected by evaluation of coat color or the like from a group of individuals obtained by cross-breeding of such chimera individuals with normal individuals. The resulting individuals are normally individuals with heterozygous insufficient expression of the receptor protein of the present invention, and they can be bred with other individuals having heterozygous insufficient expression of the receptor protein of the present invention and individuals with homozygous insufficient expression of the receptor protein of the present invention obtained from their offspring.

When egg cells are used, for example transgenic non-human mammals can be obtained having a targeting vector introduced into chromosomes by microinjection of DNA solution into the egg cell nuclei, and those with mutations in the DNA locus of the present invention due to homologous gene recombination can be selected by comparison with these transgenic non-human mammals.

Individuals having the DNA of the present invention knocked out in this way, including individual animals obtained by cross breeding, can be successively bred in a normal environment after confirmation that the DNA has been knocked out.

Moreover, normal methods can be followed for obtaining and maintaining a reproductive series. Namely, homozygous animals having inactivated DNA in both homologous chromosomes can be obtained by cross breeding male and female animals having the inactivated DNA. The resulting homozygous animals can be obtained efficiently by breeding to 1 normal individual and several homozygotes per mother. Successive generations of homozygous and heterozygous animals having inactivated DNA can be bred by cross breeding heterozygous males and females.

Non-human mammalian embryonic stem cells wherein the DNA of the present invention is inactivated are extremely useful for creating non-human mammals with insufficient expression of the DNA of the present invention.

Moreover, because they lack various biological activities which are induced by the receptor protein of the present invention, non-human mammals with insufficient expression of the DNA of the present invention can be models for diseases caused by inactivation of the biological activity of the receptor protein of the present invention, and are therefor useful for discovering causes for these diseases and investigating therapy methods.

(14a) Method of Screening Compounds having Therapeutic or Preventative Effects Against Diseases Stemming from Defects or Damage of the DNA of the Present Invention or the like

Non-human animals having insufficient expression of the DNA of the present invention can be used for screening compounds having therapeutic or preventative effects against diseases stemming from defects or damage of the DNA of the present invention.

Namely, the present invention provides a method of screening compounds having therapeutic or preventative effects against diseases stemming from defects or damage of the DNA of the present invention, characterized in that a test compound is administered to non-human mammals having insufficient expression of the DNA of the present invention, and changes in the animals are observed and measured.

The non-human animals having insufficient expression of the DNA of the present invention used in this screening method may be the same as described above.

Examples of test compounds include for example peptides, proteins, non-peptide compounds, synthetic compounds, fermentation products, cell extracts, plant extracts, animal tissue extracts, plasma and the like, and these compounds may be novel compounds or well-known compounds.

Specifically, non-human animals having insufficient expression of the DNA of the present invention can be treated with the test compound and compared with untreated control animals, and changes in the organs, tissues, disease symtpoms and the like of the animals can be used as an indicator for testing the therapeutic and preventative effects of the test compound.

Methods that can be used for treating the test animals with the test compound include oral administration and intravenous injection, and can be selected as appropriate according to the symptoms of the test animal, the nature of the test compound and the like. The dosage of the test compound can be selected as appropriate depending on the method of administration, the nature of the test compound and the like.

In this screening method, if a test compound is administered to a test animal and the test animal's blood sugar level falls by about 10% or more or preferably 30% or more or more preferably 50% or more, the test compound can be selected as a compound having therapeutic or preventative effects for the aforementioned disorder.

Compounds obtained using this screening method are compounds selected from the aforementioned test compounds, and can be used as drugs such as safe, low-toxicity therapeutic or preventative agents for disorders caused by defects or damage of the receptor protein of the present invention or the like (such as therapeutic and/or treatment drugs for prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency or calculus, or birth inducers or the like). Moreover, compounds derived from compounds obtained by this screening method can be used in the same way.

Compounds obtained by this screening method may also be in the form of salts, and salts of physiologically allowable acids (such as inorganic acids, organic acids and the like) or bases (such as alkaline metals and the like) and the like can be used as salts of these compounds, with physiologically allowable acid-added salts being particularly desirable. Such salts, which can be used, include for example salts of inorganic acids (such as hydrochloric acid, phosphoric acid, hydrobromic acid, sulfuric acid and the like) or organic acids (such as acetic acid, formic acid, propionic acid, fumaric acid, maleic acid, succinic acid, tartaric acid, citric acid, malic acid, oxalic acid, benzoic acid, methanesulfonic acid, benzenesulfonic acid or the like).

A drug containing a compound or salt thereof obtained by this screening method can be manufactured in the same way as the drug described above containing a compound which alters the binding properties of the receptor protein of the present invention with a ligand.

Because a preparation obtained in this way is safe and of low toxicity, it can be administered for example to humans or mammals (such as rats, mice, guinea pigs, rabbits, sheep, pigs, cows, horses, cats, dogs, monkeys and the like).

The dosage of this compound or salt thereof differs depending on the target condition, subject of administration, administration route and the like, but for example in the case of oral administration it is generally about 0.1 to 100 mg or preferably about 1.0 to 50 mg or more preferably about 1.0 to 20 mg per day for example in the case of a prostate cancer patient (weight 60 kg). In the case of parenteral administration the single dose will differ depending on the subject of administration, target organ, symptoms, method of administration and the like, but for example in injection form it is normally desirable to administer about 0.01 to 30 mg or preferably about 0.1 to 20 mg or more preferably about 0.1 to 10 mg a day for example by intravenous injection to a prostate cancer patient (weight 60 kg). In the case of other animals, a dose converted from the 60 kg dose can be administered.

(14b) Method for Screening Compounds, which Promote or Inhibit the Activity of a Promoter for the DNA of the Present Invention

The present invention provides a method of screening compounds or salts thereof which promote or inhibit the activity of a promoter for the DNA of the present invention, characterized in that a test compound is administered to non-human mammals with insufficient expression of the DNA of the present invention, and the expression of a reporter gene is detected.

Of the non-human mammals with insufficient expression of the DNA of the present invention described above, the non-human mammals with insufficient expression of the DNA of the present invention used in the aforementioned screening method are those in which the DNA of the present invention is inactivated by introduction of a reporter gene, and in which this reporter gene can be expressed under the control of a promoter for the DNA of the present invention.

The test compounds may be as described above.

Reporter genes such as those described above may be used, and the beta-galactosidase gene (lacZ), soluble alkaliphosphatase gene, luciferase gene or the like is desirable.

In non-human mammals with insufficient expression of the DNA of the present invention in which the DNA of the present invention has been replaced by a reporter gene, because the reporter gene is under the control of a promoter for the DNA of the present invention, the activity of the promoter can be detected by tracing the expression of a substance encoded by the reporter gene.

For example, if part of the DNA region encoding the receptor protein of the present invention has been replaced by an E. coli-derived beta-galactosidase gene (lacZ), beta-galactosidase is expressed in place of the receptor protein of the present invention in tissues in which the receptor protein of the present invention would originally have been expressed. Consequently, the expression of the receptor protein of the present invention in live animals can easily be observed by staining with a reagent such as 5-bromo4-chloro-3-indolyl-beta-galactopyranoside (X-gal), which provides a substrate for beta-galactosidase. Specifically, mice lacking the receptor protein of the present invention or tissue slices thereof can be fixed in glutaraldehyde and washed in phosphate buffered saline (PBS), then reacted for about 30 minutes to 1 hour at room temperature or near 37° C. with a stain containing X-gal, after which the tissue specimens are washed in a 1 mM EDTA/PBS solution to arrest the beta-galatosidase reaction, and coloration is observed. The mRNA encoding lacZ can also be detected by ordinary methods.

Compounds or salts thereof obtained by the aforementioned screening method are compounds selected from the aforementioned test compounds, and are compounds, which promote or inhibit promoter activity for the DNA of the present invention.

Compounds obtained by this screening method may form salts, and salts of these compounds, which can be used, include salts with physiologically acceptable acids (such as inorganic acids and the like) and bases (such as organic acids and the like), with physiologically acceptable acid-added salts being particularly desirable. Such salts which can be used include for example salts with inorganic acids (such as hydrochloric acid, phosphoric acid, hydrobromic acid, sulfuric acid and the like) and salts with organic acids (such as acetic acid, formic acid, propionic acid, fumaric acid, maleic acid, succinic acid, tartaric acid, citric acid, malic acid, oxalic acid, benzoic acid, methanesulfonic acid, benzenesulfonic acid or the like).

Since compounds and salts thereof which promote promoter activity for the DNA of the present invention are capable of promoting expression of the receptor protein of the present invention and promoting the function of the receptor protein, they are useful for example as drugs such as preventative and/or therapeutic drugs for disorders associated with dysfunction of the receptor protein of the present invention for example.

Since compounds and salts thereof which inhibit promoter activity for the DNA of the present invention are capable of inhibiting expression of the receptor protein of the present invention and inhibiting the function of the receptor protein, they are useful for example as drugs such as preventative and/or therapeutic drugs for disorders associated with over-expression of the receptor protein of the present invention.

Specifically, compounds which promote or inhibit promoter activity for the DNA of the present invention are useful for example as birth inducers or as preventative and/or therapeutic drugs for prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency, calculus and the like.

Moreover, compounds derived from compounds obtained by the aforementioned screening method can be used in the same way.

A drug containing a compound or salt thereof obtained by this screening method can be manufactured in the same way as the aforementioned drug containing a compound which alters the binding properties of the receptor protein or salt thereof of the present invention with a ligand.

Because a preparation obtained in this way is safe and of low toxicity, it can be administered for example to humans and other mammals (such as rats, mice, guinea pigs, rabbits, sheep, pigs, cows, horses, cats, dogs, monkeys and the like).

The dosage of this compound or salt thereof differs depending on the target condition, subject of administration, administration route and the like, but for example in the case of oral administration of a compound which promotes or inhibits promoter activity for the DNA of the present invention, it is generally about 0.1 to 100 mg or preferably about 1.0 to 50 mg or more preferably about 1.0 to 20 mg per day for example in the case of a prostate cancer patient (weight 60 kg). In the case of parenteral administration the single dose will differ depending on the subject of administration, target organ, symptoms, method of administration and the like, but for example in injection form it is normally desirable to administer about 0.01 to 30 mg or preferably about 0.1 to 20 mg or more preferably about 0.1 to 10 mg a day by intravenous injection to a prostate cancer patient (weight 60 kg). In the case of other animals, a dose converted from the 60 kg dose can be administered.

In this way, non-human mammals having insufficient expression of the DNA of the present invention are extremely useful for screening compounds or salts thereof which promote or inhibit the activity of a promoter for the DNA of the present invention, and can contribute greatly to discovering the causes of various disorders stemming from insufficient expression of the DNA of the present invention, or to the development of preventative and/or therapeutic drugs.

Moreover, by using DNA containing a promoter region for the receptor protein of the present invention, attaching genes encoding various proteins downstream therefrom and injecting it into egg cells of animals to create so-called transgenic animals, it is possible to specifically induce synthesis of the receptor protein and study its effects in vivo. Moreover, if a suitable reporter gene is bound to the aforementioned promoter part and a cell line such as that expressed thereby is established, it can be used as a search system for low molecular weight compounds which act to specifically promote or inhibit the productive ability of the receptor protein of the present invention itself in vivo.

It was also discovered using the aforementioned method for analyzing gene expression of the present invention that the EDG-1 receptor is highly expressed in vascular endothelial cells and the EDG-2 receptor is highly expressed in vascular smooth muscle cells. Consequently, vascular endothelial cells are useful for screening EDG-1 receptor agonists or antagonists, particularly EDG-1 receptor agonists which can be preventative and/or therapeutic drugs for arteriosclerosis, myocardial infarction, cerebral infarction or ischemic disease, while vascular smooth muscle cells are useful for screening EDG-2 receptor agonists or antagonists, particularly EDG-2 receptor antagonists which can be preventative and/or therapeutic drugs for arteriosclerosis, myocardial infarction, cerebral infarction or ischemic disease.

A screening method for an EDG-1 receptor agonist or antagonist or an EDG-2 receptor agonist or antagonist of the present invention is explained below.

The EDG-1 receptor used in the present invention is a receptor comprising an amino acid sequence identical to or substantially identical to the amino acid sequence represented by SEQ ID NO: 38. The EDG-1 receptor having the amino acid sequence represented by SEQ ID NO: 38 has the same amino acid sequence as the protein described under Genbank Accession Number AAA52336.

The EDG-2 receptor used in the present invention is a receptor comprising an amino acid sequence identical to or substantially identical to the amino acid sequence represented by SEQ ID NO: 40. The EDG-2 receptor having the amino acid sequence represented by SEQ ID NO: 40 has the same amino acid sequence as the protein described under Genbank Accession Number U80811.

A “substantially identical amino acid sequence” signifies an amino acid sequence having for example about 50% or greater or preferably about 60% or greater or more preferably about 70% or greater or even more preferably about 80% or greater or still more preferably about 90% or greater or most preferably about 95% or greater homology with the compared amino acid sequence.

A protein comprising an amino acid sequence substantially identical to the amino acid sequence represented by SEQ ID NO: 38 and having substantially the same activity as an EDG-1 receptor comprising the amino acid sequence represented by SEQ ID NO: 38 or the like for example is preferred as the protein comprising an amino acid sequence substantially identical to the amino acid sequence represented by SEQ ID NO: 38.

A protein comprising an amino acid sequence substantially identical to the amino acid sequence represented by SEQ ID NO: 40 and having substantially the same activity as an EDG-2 receptor comprising the amino acid sequence represented by SEQ ID NO: 40 or the like for example is preferred as the protein comprising an amino acid sequence substantially identical to the amino acid sequence represented by SEQ ID NO: 40.

Examples of substantially the same activity include for example ligand binding activity, signal transmission and the like. Substantially the same means that the activities are the same in character. Consequently, activity such as ligand binding activity, signal transmission and the like is preferably equivalent (such as about 0.01 to 100 times or more preferably 0.5 to 20 times or more preferably 0.5 to 2 times), but quantitative factors such as the degree of activity and the molecular weight of the protein may be different.

Measurement of activity such as ligand binding activity and signal transmission can be accomplished by well-known methods, and for example they can be measured according to the ligand determination method or screening method described below.

A protein comprising (1) an amino acid sequence having one or two or more (preferably about 1 to 30 or more preferably about 1 to 10 or still more preferably several (1 to 5)) amino acids deleted from the amino acid sequence represented by SEQ ID NO: 38, (2) an amino acid sequence having one or two or more (preferably about 1 to 30 or more preferably about 1 to 10 or still more preferably several (1 to 5)) amino acids added to the amino acid sequence represented by SEQ ID NO: 38, (3) an amino acid sequence having one or two or more (preferably about 1 to 30 or more preferably about 1 to 10 or still more preferably several (1 to 5)) amino acids replaced by other amino acids in the amino acid sequence represented by SEQ ID NO: 38, or (4) an amino acid sequence which is a combination of these or the like can be used as the EDG-1 receptor.

A protein comprising (1) an amino acid sequence having one or two or more (preferably about 1 to 30 or more preferably about 1 to 10 or still more preferably several (1 to 5)) amino acids deleted from the amino acid sequence represented by SEQ ID NO: 40, (2) an amino acid sequence having one or two or more (preferably about 1 to 30 or more preferably about 1 to 10 or still more preferably several (1 to 5)) amino acids added to the amino acid sequence represented by SEQ ID NO: 40, (3) an amino acid sequence having one or two or more (preferably about 1 to 30 or more preferably about 1 to 10 or still more preferably several (1 to 5)) amino acids replaced by other amino acids in the amino acid sequence represented by SEQ ID NO: 40, or (4) an amino acid sequence which is a combination of these can be used as the EDG-2 receptor.

Following peptide marking conventions, the left end of the EDG-1 receptor and EDG-2 receptor is the N terminus (amino terminus) and the right end is the C terminus (carboxyl terminus). The C terminus of the receptor protein of the present invention may be a carboxyl group (—COOH), carboxylate (—COO⁻), amide (—CONH₂) or ester (—COOR).

A methyl, ethyl, n-propyl, isopropyl or n-butyl or other C₁₋₆ alkyl group for example, a cyclopentyl, cyclohexyl or other C₃₋₈ cycloalkyl group for example, a phenyl, alpha-naphthyl or other C₆₋₁₂ aryl group for example, a benzyl, phenethyl or other phenyl-C₁₋₂ alkyl group for example or an alpha-naphthylmethyl or other alpha-naphthyl-C₁₋₂ alkyl group or other C₇₋₁₄ aralkyl group or else a pivaloyl oxymethyl group or the like widely used as an ester for oral use can be used as the R in the ester here.

When the EDG-1 receptor and EDG-2 receptor have carboxyl groups (or carboxylates) somewhere other than the C terminus, they are included as EDG-1 and EDG-2 receptors if the carboxyl group is amidified or esterified. The aforementioned ester of the C terminus for example can be used as the ester in this case.

The aforementioned EDG-1 receptor and EDG-2 receptor in which the amino group of the methionine residue of the N terminus is protected by a protective group (such as a formyl group, acetyl or other C₂₋₄ alkanoyl or other C₁₋₆ acyl group or the like), in which a glutamyl group produced by nicking of the N end in vivo is pyroglutaminated, or in which a substitutional group (such as an —OH, —SH, amino group, imidazole group, indole group, guanidino group or the like) on a side chain of an amino acid in the molecule is protected by a suitable protective group (such as a formyl group, acetyl or other C₂₋₆ alkanoyl or other C₁₋₆ acyl group or the like) are included as the EDG-1 and EDG-2 receptor, as are composite proteins such as so-called glycoproteins having bound sugar chains.

Specific examples of the EDG-1 receptor include for example a receptor protein consisting of the amino acid sequence represented by SEQ ID NO: 38 and the like.

Specific examples of the EDG-2 receptor include for example a receptor protein consisting of the amino acid sequence represented by SEQ ID NO: 40 and the like.

The method for screening EDG-1 receptor agonists and antagonists of the present invention is characterized by the use of vascular endothelial cells in which expression of the EDG-1 receptor is extremely high in comparison with other cells or tissues.

Similarly, the method of screening EDG-2 receptor agonists and antagonists of the present invention is characterized by the use of vascular smooth muscle cells in which expression of the EDG-2 receptor is extremely high in comparison with other cells or tissues.

There are no particular limits on ligands for the EDG-1 receptor as long as they are compounds which bind to the EDG-1 receptor, but for example Sphingosine-1-phosphate (S1P) and the like can be used. A compound, which binds to the EDG-1 receptor, is abbreviated simply as EDG-1.

There are no particular limits on ligands for the EDG-2 receptor as long as they are compounds, which bind to the EDG-2 receptor, but for example lysophosphatidic acid (LPA) and the like can be used. A compound, which binds to the EDG-2 receptor, is abbreviated simply as EDG-2.

Specifically, the screening method of the present invention provides:

(1) Method A for screening EDG-1 receptor agonists or antagonists, characterized by a comparison of (i) bringing EDG-1 into contact with vascular endothelial cells and (ii) bringing EDG-1 and a test compound into contact with vascular endothelial cells; and

(2) Method B for screening EDG-2 receptor agonists or antagonists, characterized by a comparison of (i) bringing EDG-2 into contact with vascular smooth muscle cells and (ii) bringing EDG-2 and a test compound into contact with vascular smooth muscle cells.

Screening method A of the present invention is characterized in that activity which promotes or activity which suppresses arachidonic acid release, acetylcholine release, intercellular Ca²⁺ release, intercellular cAMP production, intercellular cAMP suppression, intercellular cGMP production, inositolphosphoric acid production, cell membrane potential fluctuation, phosphorylation of intercellular proteins, activation of c-fos, cell proliferation, carbon monoxide production, wandering activity, low molecular weight G protein Rho and Rac activation, phosphatidyl inositol (PI) 3 kinase activity, pH reduction or the like for example in vascular endothelial cells is measured and compared in cases (i) and (ii).

Screening method B of the present invention is characterized in that activity which promotes or activity which suppresses arachidonic acid release, acetylcholine release, intercellular Ca²⁺ release, intercellular cAMP production, intercellular cAMP suppression, intercellular cGMP production, inositolphosphoric acid production, cell membrane potential fluctuation, phosphorylation of intercellular proteins, activation of c-fos, cell proliferation, carbon monoxide production, wandering activity, low molecular :weight G protein Rho and Rac activation, phosphatidyl inositol (PI) 3 kinase activity, pH reduction or the like for example in vascular smooth muscle cells is measured and compared in cases (i) and (ii).

More specifically, screening method A of the present invention is:

(1a) A method of screening EDG-1 receptor agonists wherein the intercellular cAMP suppression, intercellular cGMP production, inositolphosphoric acid production, cell membrane potential fluctuation, phosphorylation of intercellular proteins, activation of c-fos, cell proliferation, carbon monoxide production, wandering activity, low molecular weight G protein Rho and Rac activation, phosphatidyl inositol (PI) 3 kinase activity and pH reduction of vascular endothelial cells are measured when a test compound is brought into contact with the vascular endothelial cells, and the test compound is selected if the aforementioned activity rises by for example 10% or more or preferably 30% or more or particularly preferably 50% or more;

(1b) A method of screening EDG-l receptor agonists wherein the intercellular cAMP suppression, intercellular cGMP production, inositolphosphoric acid production, cell membrane potential fluctuation, phosphorylation of intercellular proteins, activation of c-fos, cell proliferation, carbon monoxide production, wandering activity, low molecular weight G protein Rho and Rac activation, phosphatidyl inositol (PI) 3 kinase activity and pH reduction of vascular endothelial cells are measured in a case in which EDG-1 and a test compound are brought into contact with the vascular endothelial cells in comparison with a case in which EDG-1 is brought into contact with the vascular endothelial cells, and the test compound is selected if the aforementioned activity rises by for example 10% or more or preferably 30% or more or particularly preferably 50% or more; or

(1c) A method of screening EDG-1 receptor antagonists wherein the intercellular cAMP suppression, intercellular cGMP production, inositolphosphoric acid production, cell membrane potential fluctuation, phosphorylation of intercellular proteins, activation of c-fos, cell proliferation, carbon monoxide production, wandering activity, low molecular weight G protein Rho and Rac activation, phosphatidyl inositol (PI) 3 kinase activity and pH reduction of vascular endothelial cells are measured in a case in which EDG-1 and a test compound are brought into contact with the vascular endothelial cells in comparison with a case in which EDG-1 is brought into contact with the vascular endothelial cells, and the test compound is selected if the aforementioned activity decreases by 10% or more or preferably 30% or more or particularly preferably 50% or more.

Similarly, screening method B of the present invention is:

(2a) A method of screening EDG-2 receptor agonists wherein the intercellular cAMP suppression, intercellular cGMP production, inositolphosphoric acid production, cell membrane potential fluctuation, phosphorylation of intercellular proteins, activation of c-fos, cell proliferation, carbon monoxide production, wandering activity, low molecular weight G protein Rho and Rac activation, phosphatidyl inositol (PI) 3 kinase activity and pH reduction of vascular smooth muscle cells are measured when a test compound is brought into contact with the vascular smooth muscle cells, and the test compound is selected if the aforementioned activity rises by for example 10% or more or preferably 30% or more or particularly preferably 50% or more;

(2b) A method of screening EDG-2 receptor agonists wherein the intercellular cAMP suppression, intercellular cGMP production, inositolphosphoric acid production, cell membrane potential fluctuation, phosphorylation of intercellular proteins, activation of c-fos, cell proliferation, carbon monoxide production, wandering activity, low molecular weight G protein Rho and Rac activation, phosphatidyl inositol (PI) 3 kinase activity and pH reduction of vascular smooth muscle cells are measured in a case in which EDG-1 and a test compound are brought into contact with the vascular smooth muscle cells in comparison with a case in which EDG-1 is brought into contact with the vascular smooth muscle cells, and the test compound is selected if the aforementioned activity rises by for example 10% or more or preferably 30% or more or particularly preferably 50% or more; or

(2c) A method of screening EDG-2 receptor antagonists wherein the intercellular cAMP suppression, intercellular cGMP production, inositolphosphoric acid production, cell membrane potential fluctuation, phosphorylation of intercellular proteins, activation of c-fos, cell proliferation, carbon monoxide production, wandering activity, low molecular weight G protein Rho and Rac activation, phosphatidyl inositol (PI) 3 kinase activity and pH reduction of vascular smooth muscle cells are measured in a case in which EDG-2 and a test compound are brought into contact with the vascular smooth muscle cells in comparison with a case in which EDG-2 is brought into contact with the vascular smooth muscle cells, and the test compound is selected if the aforementioned activity decreases by 10% or more or preferably 30% or more or particularly preferably 50% or more.

Test compounds which can be used include for example peptides, proteins, non-peptide compounds, synthetic compounds, fermentation products, cell extracts, plant extracts, animal tissue extracts and the like, and these compounds may be either novel compounds or well-known compounds.

It is also preferable that a compound which alters the binding properties of EDG-1 with EDG-1 receptors be used as the test compound in screening method A, and that screening method A be used as secondary evaluation system for this compound.

It is also preferable that a compound, which alters the binding properties of EDG-2 with EDG-2 receptors be used as the test compound in screening method B, and that screening method B be used as secondary evaluation system.

Screening of compounds, which alter the binding properties of EDG-1 with EDG-1 receptors and compounds, which alter the binding properties of EDG-2 with EDG-2 receptors can be accomplished in accordance with the methods described above for screening compounds which alter the binding properties of the dJ287G14.2 receptor protein with ligands.

The screening method of the present invention is explained in detail below.

Vascular endothelial cells or vascular smooth muscle cells can be prepared from the blood vessels of humans and other mammals (such as guinea pigs, rats, mice, rabbits, pigs, sheep, cows, monkeys and the like) in accordance with well-known methods.

Culture of vascular endothelial cells can be accomplished for example according to the methods described in Cell Oct. 29, 1999; 99(3): 301-12, or methods conforming thereto.

Culture of vascular smooth muscle cells can be accomplished for example according to the methods described in Cardiovasc. Res. Sep. 32, 1996(3): 516-23, or methods conforming thereto.

Specifically, to implement the screening method of the present invention, activity which promotes or activity which suppresses the arachidonic acid release, acetylcholine release, intercellular Ca²⁺ release, intercellular cAMP production, intercellular cAMP suppression, intercellular cGMP production, inositolphosphoric acid production, cell membrane potential fluctuation, phosphorylation of intercellular proteins, c-fos activation, pH reduction and the like of vascular endothelial cells or vascular smooth muscle cells can be measured using well-known methods or a commercial measurement kit.

Specifically, vascular endothelial cells or vascular smooth muscle cells are first cultured on multi-well plates or the like. Fresh medium or a suitable buffer exhibiting no toxicity towards the cells is substituted ahead of time in preparation for screening, a test compound or the like is added, and after a fixed incubation time the cells are extracted or supernatant is collected and the resulting products are assayed according to the respective methods therefor. If production of substances, which are markers for cell stimulus activity (for example, arachidonic acid and the like), is difficult to test because of degradation enzymes contained in the cells, an inhibitor for these degradation enzymes may be added for purposes of the assay. Activity such as cAMP production inhibition or the like can be detected as production inhibition action with respect to cells in which basic production is increased with forskolin or the like.

A screening kit for EDG-1 receptor agonists or antagonists or EDG-2 receptor agonists or antagonists, is one, which contains vascular endothelial cells or vascular smooth muscle cells.

The following can be given as an example of a screening kit of the present invention.

1. Screening Reagents

(1) Measurement Buffer and Washing Buffer

0.05% bovine serum albumin (Sigma) added to Hanks' Balanced Salt Solution (Gibco).

May be filtered and sterilized with an 0.45 μm filter and stored at 4° C., or prepared as needed. (2) Vascular Endothelial Cells or Vascular Smooth Muscle Cells

Vascular endothelial cells or vascular smooth muscle cells are passaged 5×10⁵/well on 12-well plates, and cultured for 2 days at 37° C. in 5% CO₂, 95% air.

(3) Ligand Standard Solution

EDG-1 or EDG-2 is dissolved to 1 mM in PBS containing 0.1% bovine serum albumin (Sigma), and stored at −20° C.

Because an EDG-1 receptor agonist obtained using screening method A or a screening kit of the present invention has an action which is the same as the physiological activity of EDG-1, it is useful as a safe, low toxicity drug corresponding to this physiological activity.

Because an EDG-1 receptor antagonist obtained using screening method A or a screening kit of the present invention can suppress the physiological activity of EDG-1, it is useful as a safe, low toxicity drug which suppresses this physiological activity.

Because an EDG-2 receptor agonist obtained using screening method A or a screening kit of the present invention has an action which is the same as the physiological activity of EDG-2, it is useful as a safe, low toxicity drug corresponding to this physiological activity.

Because an EDG-2 receptor antagonist obtained using screening method B or a screening kit of the present invention can suppress the physiological activity of EDG-2, it is useful as a safe, low toxic drug, which suppresses this physiological activity.

Specifically, EDG-1 receptor agonists and EDG-2 receptor antagonists obtained using a screening method or screening kit of the present invention are useful as preventative and/or therapeutic drugs for disorders such as arteriosclerosis, cardiac infarction, cerebral infarction, ischemic disease and the like.

When an EDG-1 receptor agonist or antagonist or EDG-2 receptor agonist or antagonist is used as a preventative and/or therapeutic agent for the aforementioned disorders, it can be formulated in the same way as the aforementioned drug containing a compound which alters the binding properties of the dJ287G14.2 receptor protein with a ligand.

Because a preparation obtained in this way is safe and of low toxicity, it can be administered for example to humans or mammals (such as rats, mice, guinea pigs, rabbits, sheep, pigs, cows, horses, cats, dogs, monkeys and the like).

The dosage of the EDG-1 receptor agonist or EDG-2 receptor antagonist differs depending on the target disease, subject of administration, administration route and the like, but for example in the case of oral administration of an EDG-1 receptor agonist or EDG-2 receptor antagonist it is generally about 0.1 to 100 mg or preferably about 1.0 to 50 mg or more preferably about 1.0 to 20 mg a day for example in the case of an arteriosclerosis patient (weight 60 kg). In the case of parenteral administration, the single dose will differ depending on the subject of administration, target organ, symptoms and administration method and the like, but for example in injection form it is normally desirable to administer about 0.01 to 30 mg or preferably about 0.1 to 20 mg or more preferably about 0.1 to 10 mg a day by intravenous injection to an arteriosclerosis patient (weight 60 kg) for example. In the case of other animals, a dose converted from the 60 kg dose can be administered.

(Identification of Abbreviations)

When bases, amino acids, compounds and the like are represented by abbreviations in these specifications, representation is based on the abbreviations of the IUPAC-IUB Commission on Biochemical Nomenclature or on conventional abbreviations used in the field, and some examples are given below. When an amino acid may have optical isomers, the L form is indicated unless otherwise specified.

| | |
|---|---|
| DNA | Deoxyribonucleic acid |
| cDNA | Complementary deoxyribonucleic acid |
| a or A | Adenine |
| t or T | Thymine |
| g or G | Guanine |
| c or C | Cytosine |
| u or U | Uracil |
| RNA | Ribonucleic acid |
| mRNA | Messenger ribonucleic acid |
| dATP | Deoxyadenosine triphosphate |
| dTTP | Deoxythymidine triphosphate |
| dGTP | Deoxyguanosine triphosphate |
| dCTP | Deoxycytidine triphosphate |
| ATP | Adenosine triphosphate |
| Gly | Glycine |
| Ala | Alanine |
| Val | Valine |
| Leu | Leucine |
| Ile | Isoleucine |
| Ser | Serine |
| Thr | Threonine |
| Cys | Cysteine |
| Met | Methionine |
| Glu | Glutamic acid |
| Asp | Aspartic acid |
| Lys | Lysine |
| Arg | Arginine |
| His | Histidine |
| Phe | Phenylalanine |
| Tyr | Tyrosine |
| Trp | Tryptophan |
| Pro | Proline |
| Asn | Asparagine |
| Gln | Glutamine |
| pGlu | Pyroglutamic acid |

The sequences described in the sequence listing of these specifications are as follows.

[SEQ ID NO: 1] This represents the nucleotide sequence of hORL1.

[SEQ ID NO: 2] This represents the nucleotide sequence of the upstream primer M-572F used in Example 1.

[SEQ ID NO: 3] This represents the nucleotide sequence of the downstream primer M-714R used in Example 1.

[SEQ ID NO: 4] This represents the nucleotide sequence of the probe M-658T used in Example 1.

[SEQ ID NO: 5] This represents the nucleotide sequence of a primer used in Examples 2 and 3.

[SEQ ID NO: 6) This represents the nucleotide sequence of a primer used in Examples 2 and 3.

[SEQ ID NO: 7] This represents the nucleotide sequence of the probe used in Examples 2 and 3.

[SEQ ID NO: 8] This represents the nucleotide sequence of DNA encoding the human dJ287G14.2 receptor.

[SEQ ID NO: 9] This represents the amino acid sequence of the human dJ287G14.2 receptor.

[SEQ ID NO: 10] This represents the nucleotide sequence of cDNA obtained in Example 2.

[SEQ ID NO: 11] This represents the nucleotide sequence of a primer used in PCR in Reference Example 1.

[SEQ ID NO: 12] This represents the nucleotide sequence of a primer used in PCR in Reference Example 1.

[SEQ ID NO: 13] This represents the amino acid sequence of mouse dJ287G14.2 receptor A.

[SEQ ID NO: 14] This represents the nucleotide sequence of DNA encoding mouse dJ287G14.2 receptor A.

[SEQ ID NO: 15] This represents the amino acid sequence of mouse dJ287G14.2 receptor B.

[SEQ ID NO: 16] This represents the nucleotide sequence of DNA encoding mouse dJ287G14.2 receptor B.

[SEQ ID NO: 17] This represents the amino acid sequence of mouse dJ287G14.2 receptor C.

[SEQ ID NO: 18] This represents the nucleotide sequence of DNA encoding mouse dJ287G14.2 receptor C.

[SEQ ID NO: 19] This represents the amino acid sequence of mouse dJ287G14.2 receptor D.

[SEQ ID NO: 20] This represents the nucleotide sequence of DNA encoding mouse dJ287G14.2 receptor D.

[SEQ ID NO: 21] This represents the amino acid sequence of mouse dJ287G14.2 receptor E.

[SEQ ID NO: 22] This represents the nucleotide sequence of DNA encoding mouse dJ287G14.2 receptor E.

[SEQ ID NO: 23] This represents the amino acid sequence of mouse dJ287G14.2 receptor F.

[SEQ ID NO: 24] This represents the nucleotide sequence of DNA encoding mouse dJ287G14.2 receptor F.

[SEQ ID NO: 25] This represents the amino acid sequence of mouse dJ287G14.2 receptor G.

[SEQ ID NO: 26] This represents the nucleotide sequence of DNA encoding mouse dJ287G14.2 receptor G.

[SEQ ID NO: 27] This represents the amino acid sequence of mouse dJ287G14.2 receptor H.

[SEQ ID NO: 28] This represents the nucleotide sequence of DNA encoding mouse dJ287G14.2 receptor H.

[SEQ ID NO: 29] This represents the nucleotide sequence of a primer used in Example 6.

[SEQ ID NO: 30] This represents the nucleotide sequence of a primer used in Example 6.

[SEQ ID NO: 31] This represents the nucleotide sequence of a primer used in Example 6.

[SEQ ID NO: 32] This represents the nucleotide sequence of a primer used in Example 6.

[SEQ ID NO: 33) This represents the nucleotide sequence of a primer used in Example 6.

[SEQ ID NO: 34] This represents the nucleotide sequence of a primer used in Example 6.

[SEQ ID NO: 35] This represents the nucleotide sequence of a primer used in Example 6.

[SEQ ID NO: 36] This represents the nucleotide sequence of a primer used in Example 6.

[SEQ ID NO: 37] This represents the nucleotide sequence of a primer used in Example 6.

[SEQ ID NO: 38] This represents the amino acid sequence of the human EDG-1 receptor.

[SEQ ID NO: 39] This represents the nucleotide sequence of cDNA encoding the amino acid sequence of the human EDG-1 receptor.

[SEQ ID NO: 40] This represents the amino acid sequence of the human EDG-2 receptor.

[SEQ ID NO: 41] This represents the nucleotide sequence of cDNA encoding the amino acid sequence of the human EDG-2 receptor.

[SEQ ID NO: 42] This represents the amino acid sequence of the human EDG-3 receptor.

[SEQ ID NO: 43] This represents the nucleotide sequence of cDNA encoding the amino acid sequence of the human EDG-3 receptor.

[SEQ ID NO: 44] This represents the amino acid sequence of the human EDG4 receptor.

[SEQ ID NO: 45] This represents the nucleotide sequence of cDNA encoding the amino acid sequence of the human EDG-4 receptor.

[SEQ ID NO: 46] This represents the amino acid sequence of the human EDG-5 receptor.

[SEQ ID NO: 47] This represents the nucleotide sequence of cDNA encoding the amino acid sequence of the human EDG-5 receptor.

[SEQ ID NO: 48] This represents the amino acid sequence of the human EDG-6 receptor.

[SEQ ID NO: 49] This represents the nucleotide sequence of cDNA encoding the amino acid sequence of the human EDG-6 receptor.

[SEQ ID NO: 50] This represents the amino acid sequence of the human EDG-7 receptor.

[SEQ ID NO: 51] This represents the nucleotide sequence of cDNA encoding the amino acid sequence of the human EDG-7 receptor.

[SEQ ID NO: 52] This represents the amino acid sequence of the human EDG-8 receptor.

[SEQ ID NO: 53] This represents the nucleotide sequence of cDNA encoding the amino acid sequence of the human EDG-8 receptor.

[SEQ ID NO: 54] This represents the nucleotide sequence of a primer for the human EDG-1 receptor used in Example 7.

[SEQ ID NO: 55] This represents the nucleotide sequence of a primer for the human EDG-1 receptor used in Example 7.

[SEQ ID NO: 56] This represents the nucleotide sequence of a probe for the human EDG-1 receptor used in Example 7.

[SEQ ID NO: 57] This represents the nucleotide sequence of a primer for the human EDG-2 receptor used in Example 7.

[SEQ ID NO: 58] This represents the nucleotide sequence of a primer for the human EDG-2 receptor used in Example 7.

[SEQ ID NO: 59] This represents the nucleotide sequence of a probe for the human EDG-2 receptor used in Example 7.

[SEQ ID NO: 60] This represents the nucleotide sequence of a primer for the human EDG-3 receptor used in Example 7.

[SEQ ID NO: 61] This represents the nucleotide sequence of a primer for the human EDG-3 receptor used in Example 7.

[SEQ ID NO: 62] This represents the nucleotide sequence of a probe for the human EDG-3 receptor used in Example 7.

[SEQ ID NO: 63] This represents the nucleotide sequence of a primer for the human EDG4 receptor used in Example 7.

[SEQ ID NO: 64] This represents the nucleotide sequence of a primer for the human EDG4 receptor used in Example 7.

[SEQ ID NO: 65] This represents the nucleotide sequence of a probe for the human EDG4 receptor used in Example 7.

[SEQ ID NO: 66] This represents the nucleotide sequence of a primer for the human EDG-5 receptor used in Example 7.

[SEQ ID NO: 67] This represents the nucleotide sequence of a primer for the human EDG-5 receptor used in Example 7.

[SEQ ID NO: 68] This represents the nucleotide sequence of a probe for the human EDG-5 receptor used in Example 7.

[SEQ ID NO: 69] This represents the nucleotide sequence of a primer for the human EDG-6 receptor used in Example 7.

[SEQ ID NO: 70] This represents the nucleotide sequence of a primer for the human EDG-6 receptor used in Example 7.

[SEQ ID NO: 71] This represents the nucleotide sequence of a probe for the human EDG-6 receptor used in Example 7.

[SEQ ID NO: 72] This represents the nucleotide sequence of a primer for the human EDG-7 receptor used in Example 7.

[SEQ ID NO: 73] This represents the nucleotide sequence of a primer for the human EDG-7 receptor used in Example 7.

[SEQ ID NO: 74] This represents the nucleotide sequence of a probe for the human EDG-7 receptor used in Example 7.

[SEQ ID NO: 75] This represents the nucleotide sequence of a primer for the human EDG-8 receptor used in Example 7.

[SEQ ID NO: 76] This represents the nucleotide sequence of a primer for the human EDG-8 receptor used in Example 7.

[SEQ ID NO: 77] This represents the nucleotide sequence of a probe for the human EDG-8 receptor used in Example 7.

### EXAMPLES

The present invention is explained in more detail below with reference to examples, but these do not limit the scope of the present invention.

### Example 1

In this example, an mRNA sample derived from normal adult human brain is used, the presence or absence and produced amounts of mRNA derived from genes belonging to the target G protein-coupled receptor, tyrosine kinase receptor, ion channel and other gene families in the sample are assayed by TaqMan, and the expressed levels of genes belonging to the target G protein-coupled receptor, tyrosine kinase receptor, ion channel and other families are analyzed. In this example, a 384-well plate is used, and the expression of 192 genes belonging to the target G protein-coupled receptor, tyrosine kinase receptor, ion channel and other families is analyzed. Genes are selected for example from the target GPCR genes ORL; M₁; M₂; M₃; M₄; M₅; A₁; A_{2A}, A_{2B}, A₃; α1A; α1B; α1D; α2A; α2B; α2C; β1; β2; β3; AT1; AT2; BB1; BB2; bb3; B₁; B₂; CB1; CB2; CCR1; CCR2; CCR3; CCR4; CCR5; CCR6; CCR7; CCR8; CCR9; CCR10; CXCR1; CXCR2; CXCR3; CXCR4; CXCR5; CX₃CR1; XCR1; C3a; C5a; fMLP; CCK₁; CCK₂; CRF₁; CRF₂; D1; D2; D3; D4; D5; ET_{A}; ET_{B}; GAL1; GAL2; GAL3; mglu₁; mglu₂; mglu₃; mglu₄; mglu₅; mglu₆; mglu₇; mglu₈; FSH; LSH; TSH; H₁; H₂; H₃; H₄; 5-HT_{1A}; 5-HT_{1B}; 5-HT_{1D}; 5-ht_{1B}; 5-ht_{1F}; 5-HT_{2A}; 5-HT_{2F}; 5-HT_{2C}; 5-HT₃; 5-HT₄; 5-ht_{5A}; 5-ht_{5B}; 5-HT₆; 5-HT₇; BLT, CysLT₁, CysLT₂; edg1; edg2; edg3; edg4; MC₁; MC₂; MC₃; MC₄; MC₅; MT₁; MT₂; MT₃; Y₁; Y₂; Y₄; Y₅; Y₆; NTS1; NTS2; DOP; KOP; MOP; NOP; P2Y₁; P2Y₂; P2Y₄; P2Y₆; P2Y₁₁; P2Y₁₂; PPAR-α; PPAR-β; PPAR-γ; DP; FP; IP; TP; EPI; EP₁; EP₂; EP₃; EP₄; PAR 1; PAR2; PAR3; PAR4; sst₁; sst₂; sst₃; sst₄;sst₅; NK₁; NK₂; NK₃; TRH₁; TRH₂; VPAC₁; VPAC₂; PAC₁; V₁ₐ; V_{1b}; V₂; and OT; Na⁺ channels (Type I; Type II/Type IIA; Type III; SCL11/NaG; PN1; NaCh6; NaDRG; SkM1/μ1, SkM2); K⁺ channels (Kv; EAG; KQT; IRK; ROMK; GIRK; K_{ATP} and the like); Ca²⁺ channels (α1G; α1E; α1S; α1C; α1D; α1B; α1A; IP3; ryanodine receptor and the like); CL⁻ channels (GABA_{A}; GABA_{C}; glycine receptor; C1C0; C1C1; CFTR and the like); and non-selective cation channels (nAChR; 5-HT₃; NMDA; AMPA; P_{2X}ATP; CNG and the like) and others.

(1) Samples

Amplification is performed using the following samples.

First, preparation of samples for hORL1 mRNA (SEQ ID NO: 1, one type of hGPCR mRNA, GenBank Accession No. X77130) is described.

(Sample for hORL1 mRNA Assay)

Serial dilution solution of hORL1 positive control cDNA.

25 ng of total RNA derived from normal adult human brain.

The normal human adult brain in this case is known to express hORL1 mRNA.

(Amplification Primers and Detection Probe for hORL1 mRNA Assay)

Amplification of the hORL1 mRNA region was performed using upstream primer M-572F (SEQ ID NO: 2) and downstream primer M-714R (SEQ ID NO: 3). Upstream primer M572F hybridizes with the complement sequence of sites 749 through 771 in the hORL1 mRNA, while downstream primer M-714R hybridizes with the sequence of sites 869 through 891 in the hORL1 mRNA. Using these primers, a product of 143 base pairs is amplified which contains part of the full-length hORL1 mRNA sequence.

Detection is performed using M-658T (SEQ ID NO: 4). This probe hybridizes with the complement sequence of sites 835 through 860 in the hORL1 mRNA.

In order to permit TaqMan-type detection, the probe M-658T is labeled at the 5′-terminal with a fluorescein fluorescent dye (FAM: reporter), and at the 3′-terminal with a rhodamine fluorescent dye (TAMRA: quencher).

When the labeled probe is in a non-hybridizing state, the fluorescence of the reporter is suppressed by the movement phenomenon of fluorescent resonance energy. To prevent elongation of the probe by DNA polymerase during amplification, the 3′-terminus of the probe is synthesized by a phosphate block.

(Specimens for Assay of Target mRNA other Than hORL1 mRNA)

The remaining 191 types of target mRNA assay specimens are prepared in the same way as the hORL1 mRNA above.

(2) Amplification

The respective PCR amplifications are performed using a TaqMan (TM) EZ RT-PCR Kit (Applied Biosystems Japan K.K.), with a total reaction liquid volume of 20 μl. The final reagent concentrations are as follows: Sample gene, 50 mM Bicine, pH 8.2, 115 mM KOAc, 0.01 mM EDTA, 60 nM ROX, 8% (W/V) glycerol, 3 mM Mn (OAc)₂, 300 μM dATP, dGTP, dCTP, 600 μM dUTP, 900 nM of each primer, 250 nM of probe, 0.5 units AmpErase UNG, 5 units rTth DNA polymerase.

The amplification reaction is performed using an ABI PRIZM (TM) 7900 HT sequence detection system (Applied Biosystems Japan K.K.), with the following thermal cycle profile.

Thermal cycling times and temperatures
Incubation before reaction: 2 minutes at 50° C.
Reverse transcription: 30 minutes at 60° C.
Inactivation: 5 minutes at 95° C.
Denaturing & annealing/elongation: 40 cycles of 20 seconds at 94° C., 1 minute at 62° C.
   (3) Quantitative TaqMan Analysis

In the TaqMan reaction, the probe, which hybridizes with the aforementioned target sequence, is hydrolyzed from the 5′-terminal by the 5′→3′ exonuclease activity of the DNA polymerase during amplification. As a result, the reporter fluorescent dye is dissociated, and fluorescent strength increases.

Accumulation of the amplification product is measured by measuring the increase in fluorescent strength of the reporter fluorescent dye in the reaction liquid. At the same time, the fluorescent strength of a fluorescent reference (fluorescent dye: ROX) used to correct experimental errors in the reaction liquid is measured. During each amplification cycle, the aforementioned reporter fluorescent dye and reference fluorescent dye are excited by light near their wavelengths of maximum excitation, and the luminescence of the reporter fluorescent dye and reference fluorescent dye is measured near the maximum of luminescence. These frequencies are determined in advance by an ABI PRIZM (TM) 7900 HT sequence detection system, and if another detector is used appropriate frequencies should be selected.

The fluorescence measurement-values are analyzed by 7900 HT SDS Software (Applied Biosystems Japan K.K.). First, the fluorescent strength of the reporter fluorescent dye is standardized by the fluorescent strength of the reference fluorescent dye, and a standardized reporter signal (Rn) is calculated. In addition, the relatively constant mean Rn value (baseline) during the cycles of the initial PCR cycles is subtracted from Rn to give ΔRn. ΔRn is plotted against number of cycles on an amplification curve, and the number of cycles at which the analysis algorithm first detects an increase in the fluorescence signal (ΔRn) corresponding to exponential amplification of the amplification product is given as the threshold cycle (C_{T}). Specifically, in order to determine this C_{T}, the initial PCR cycles (3-15 cycles) during which the amplification product is assumed not to have reached exponential amplification were taken as the baseline for calculating the standard deviation of mean ΔRn within this cycle. Next, a value 10 times this standard deviation is defined as the threshold, and the number of cycles corresponding to this threshold value on each amplification curve is given as C_{T}.

During the exponential amplification period of the amplification product, C_{T} is proportional to the logarithm of the initial number of target copies. The accumulation of amplification product during later cycles inhibits the reaction and causes it to plateau.

(5) Results

C_{T} values such as the following are obtained for example from each sample for hORL1 mRNA assay. Each C_{T} value represents a mean value obtained from multiple (such as four) reactions.

| | (C_{T} value) |
|---|---|
| (Control samples) | |
| 10⁶ copies of hORL1 positive control | 21.1, |
| 10⁵ copies of hORL1 positive control | 24.4, |
| 10⁴ copies of hORL1 positive control | 27.5, |
| 10³ copies of hORL1 positive control | 30.8, |
| 10² copies of hORL1 positive control | 33.9, |
| 0 copies of hORL1 positive control | 40.0. |
| (Normal adult human brain sample) | |
| 25 ng of total RNA derived from | 27.2 |
| normal adult human brain | |

The calibration curve is derived from CT values obtained from amplification of a template (standard sample) of a known quantity of hORL1 cDNA positive control. Specifically, C_{T} is plotted against the initial known volume (logarithm) of the standard sample to prepare the calibration curve. The linear equation C_{T}=(Log [DNA]_{T}−Log [DNA]₀)/Log (1+e) (where [DNA]₀ is the initial concentration of target template, [DNA]_{T} is the concentration of amplification product during the C_{T} cycle, e is the mean amplification efficiency, and Log(X) is a logarithm representing the base when X is 10) is used for the approximation curve, and the parameters are set by 7900 HT SDS software.

The following calibration curve is obtained from the CT values obtained from the aforementioned control samples:
C_{T}=40.83−3.307*Log [DNA]₀

The number of copies when the aforementioned normal adult human brain-derived sample is processed is calculated using a calibration curve prepared from the template of a known control. The mRNA concentration is obtained by dividing the calculated number of copies by the size of the sample.

When the standard curve described above is used to calculate the hORL1 mRNA concentration (number of hORL1 mRNA copies per ng of total RNA) for the aforementioned tissue sample, the results are as follows:

Calculated hORL1 concentration (number of copies/ng)

| (Sample) | hORL1 concentration |
|---|---|
| 25 ng of total RNA derived from | 1.04 * 10³ |
| normal adult human brains | |

The produced amount of gene mRNA belonging to other target G protein-coupled receptor, tyrosine kinase receptor, ion channel and other gene families is assayed in the same way below. The expressed amounts of genes belonging to all target G protein-coupled receptor, tyrosine kinase receptor, ion channel and other gene families in normal adult human brain are analyzed by comparing the produced amounts of gene mRNA belonging to the various target G protein-coupled receptor, tyrosine kinase receptor, ion channel and other gene families.

### Example 2

### Expression Analysis of dJ287G14.2 in Prostate Cancer Cells

(Cells and Media)

PrEC was purchased from Takara Shuzo. LNCaP-FGC was purchased from Dainippon Pharmaceutical. PC-3 and Du145 cells were purchased from ATCC. PrEC was cultured using a prostate epithelial cell medium kit (Takara Shuzo). LNCAP-FGC was cultured in RPMI-1640 with 10% FCS (Invitrogen), PC-3 cells in Hams F12K with 10% FCS (Invitrogen), and Dul45 cells in Minimum essential medium Eagle with 10% FCS, 2 mM L-glutamine, Eagle BSS and non-essential amino acids (all Invitrogen) added.

(RNA Extraction and cDNA Synthesis)

The respective cells were cultured to the pre-confluent stage. After being stripped with 0.25% trypsin-1 mM EDTA (Invitrogen) the cells were counted, and total RNA was extracted and purified according to the Rneasy mini Kit (Qiagen) manual. First strand cDNA was synthesized from the extracted RNA according to the SuperScript II (Invitrogen) manual, dissolved as described below after ethanol sedimentation and used.

(Assay using TaqMan)

The synthesized cDNA was dissolved in TE so as to correspond to 10 mg/ml RNA, then diluted to 6.67 ng/μl in TE containing 50 μg/ml of yeast tRNA. The amplification reaction reagent was prepared to a total reaction liquid volume of 15 μl per 3.75 μl (corresponding to 25 ng of RNA) of diluted cDNA solution, using TaqMan (TM) Universal PCR Master Mix (Applied Biosystems Japan K.K.) and a TaqMan (TM) Probe Kit (Applied Biosystems Japan K.K.). The respective primers and probe used were those represented by SEQ ID NOS:5-7, which were designed using Primer Express software (Applied Biosystems) from the nucleotide sequence of the human dJ287G14.2 receptor. The final concentrations were according to the manual.

TaqMan (TM) PCR was performed with an ABI PRISM (TM) 7900HT sequence detection system (Applied Biosystems Japan), and the thermal cycles were according to the TaqMan (TM) Universal PCR Master Mix (Applied Biosystems Japan) manual.

Quantitative TaqMan analysis of the amplification product was performed using 7900HT SDS Software (Applied Biosystems Japan). The number of copies was derived using as the standard the cDNA represented by SEQ ID NO: 10, which comprises a primer amplification region designed based on the nucleotide sequence (SEQ ID NO: 8) of the human dJ287G14.2 receptor.

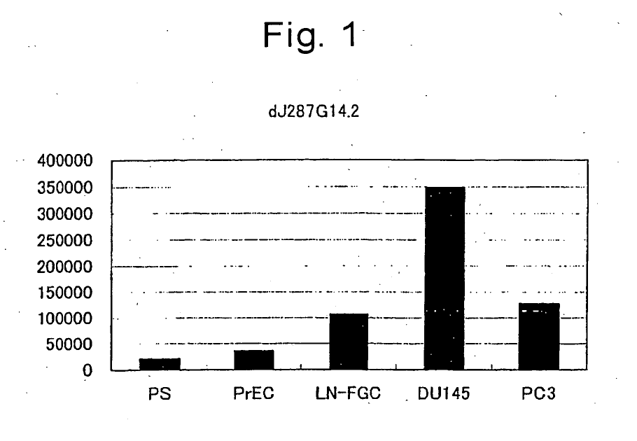

Expression of dJ287G14.2 in the respective cell lines is shown in FIG. 1.

### Example 3

### Expressed Amounts of dJ287G14.2 Receptor mRNA in Human Tissues

An ABI PRISM 7900HT (Applied Biosystems) was used for assaying expressed amounts of mRNA. The primers and probe from the human dJ287G14.2 receptor nucleotide sequence used in the assay were the same as those used in Example 2. The cDNA used as the samples was reverse transcripted using random primers from 1 μg of polyA+RNA (Clontech) derived from various human tissues. Reactions were performed using reverse transcriptase SuperScript II (GIBCO BRL) according to the attached protocols, followed by ethanol sedimentation and dissolution in 100 μl of TE. The reaction liquid for assay was prepared 20 μl per well according to the protocols for the TaqMan Universal PCR Master Mix (Applied Biosystems), with primer (0.9 μM), probe (0.25 μM) and 0.5 μl of sample cDNA added to the reaction liquid. 40 reaction cycles of 2 minutes at 50° C. and 10 minutes at 95° C. followed by 15 seconds at 95° C. and 1 minute at 60° C. were performed in the ABI PRISM 7900HT.

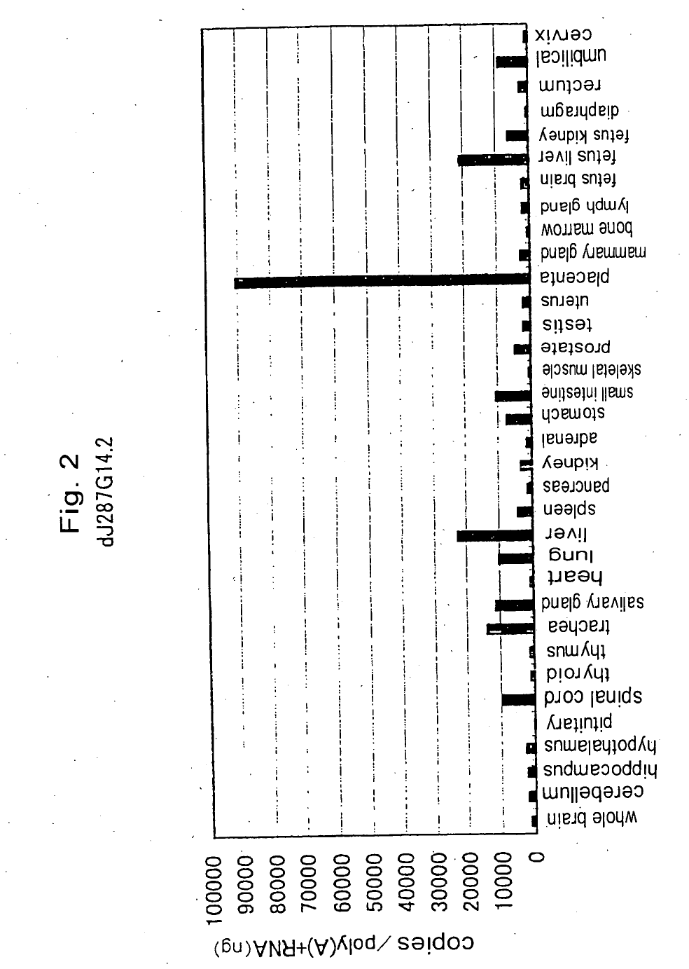

The distribution of dJ287G14.2 receptor mRNA expression in various human tissues is shown in FIG. 2. High expression was observed in the placenta, liver and the like.

### Reference Example 1

### Obtaining dJ287G14.2 Receptor Gene by PCR from cDNA of Human Prostate Cancer-Derived Cell Line LNCAP

Amplification by PCR was performed using the following two types of synthetic DNA with human prostate cancer-derived cell line LNCAP cDNA as the template.

(SEQ ID NO: 11)

| | |
|---|---|
| F1: | 5′-CTCGAGATGATGTTTCGCTCAGATCGAATGTGG-3′ |

(SEQ ID NO: 12)

| | |
|---|---|
| R1: | 5′-GCTAGCTCAGCATGGGCCAGTTTTGACAAGGAC-3′ |

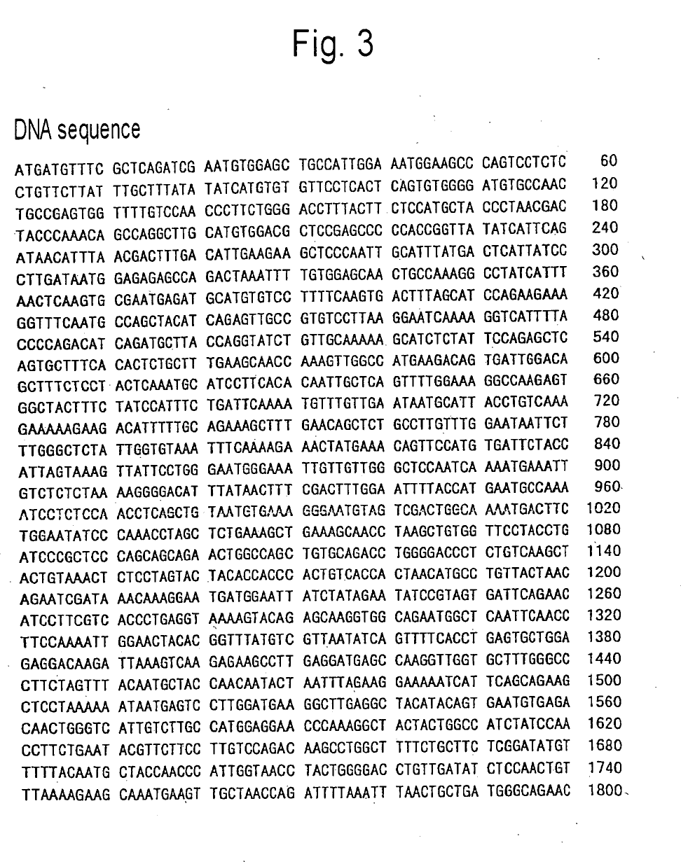

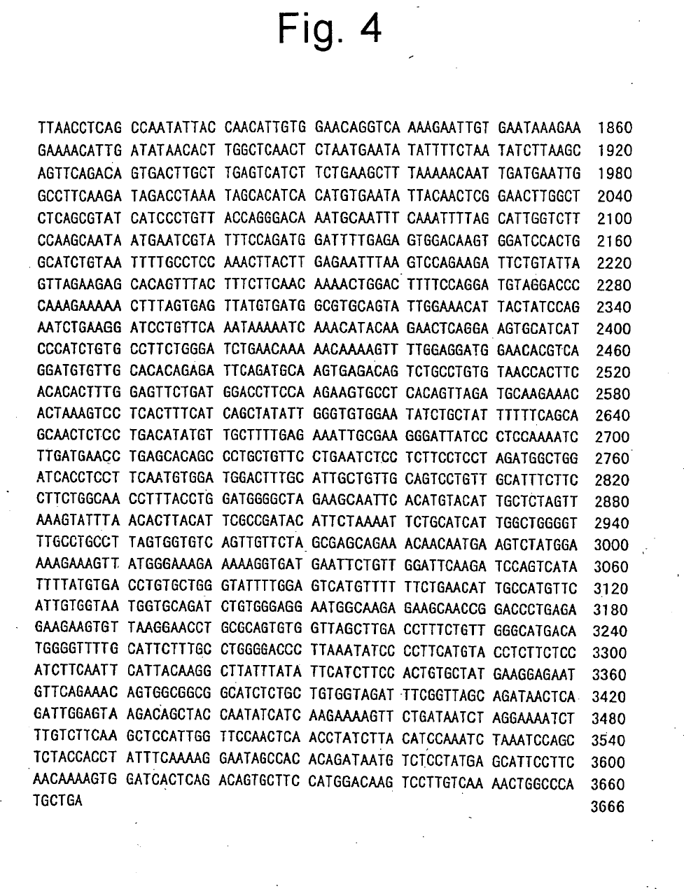

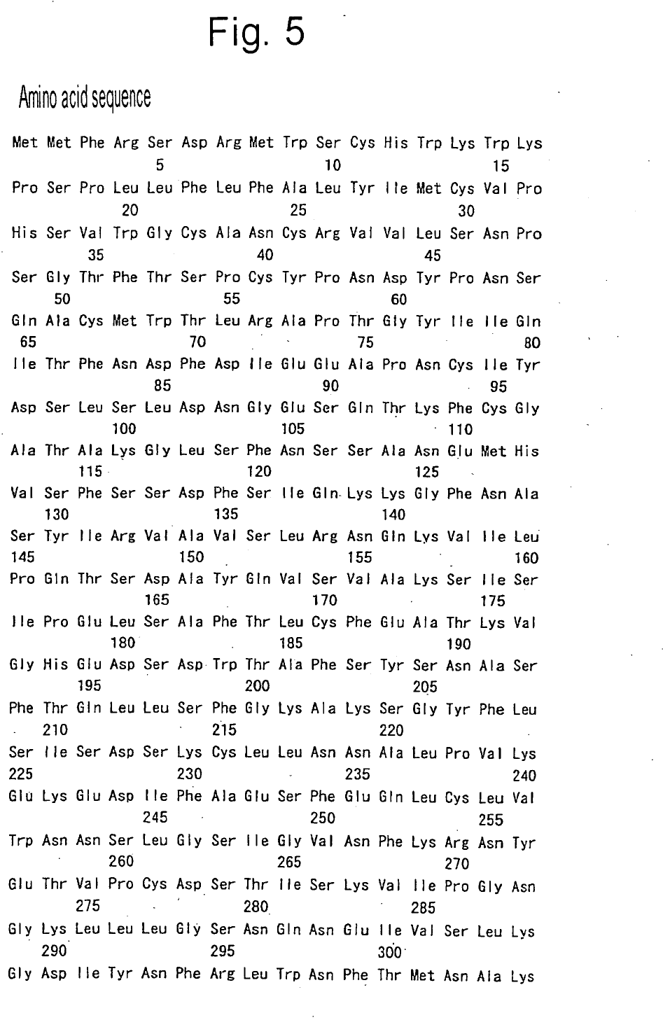

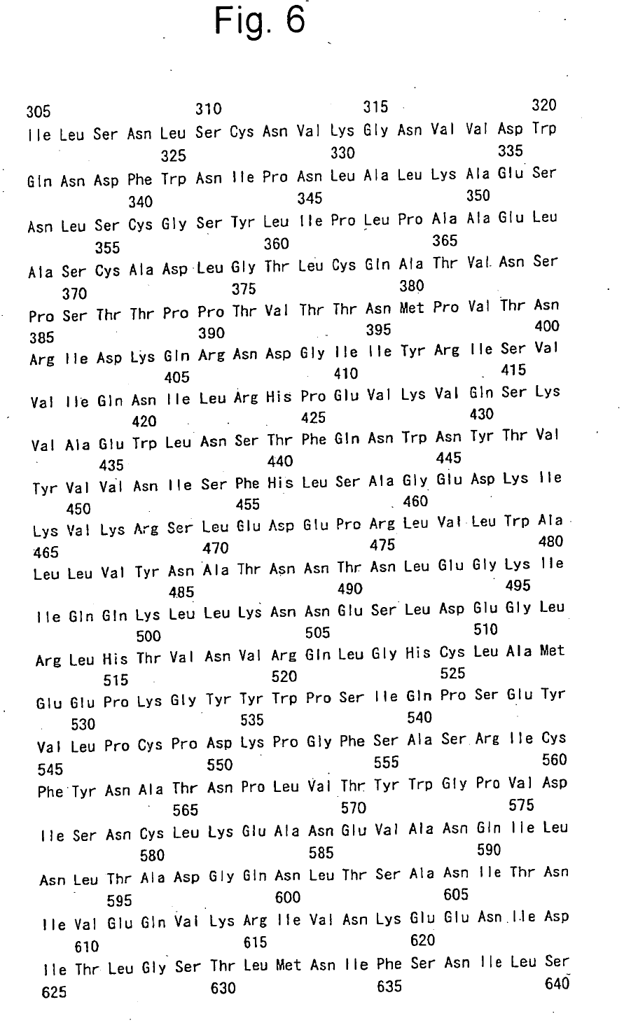

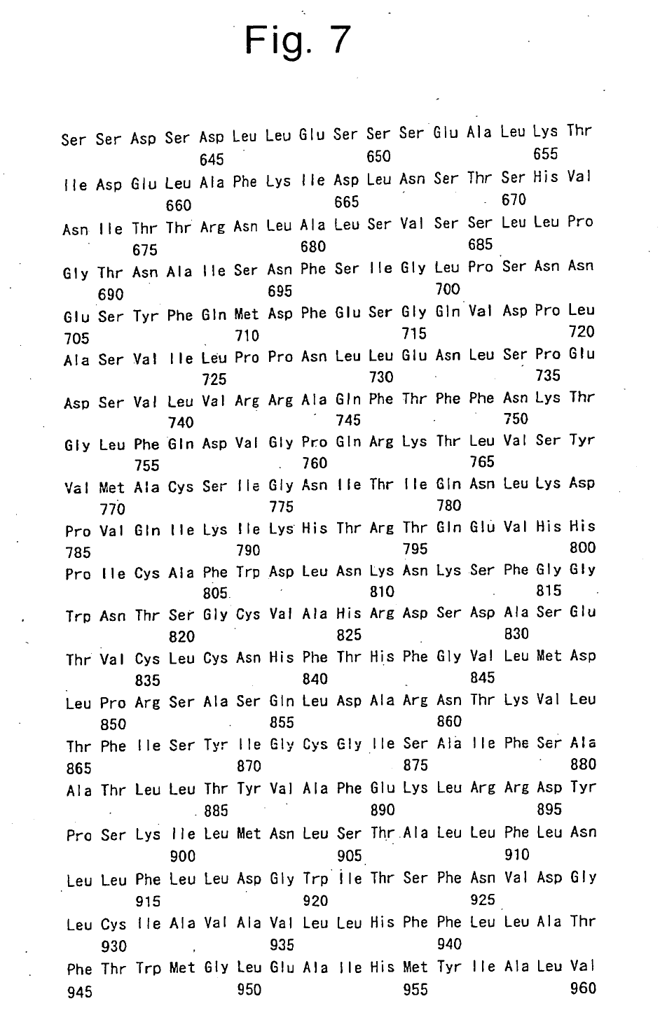

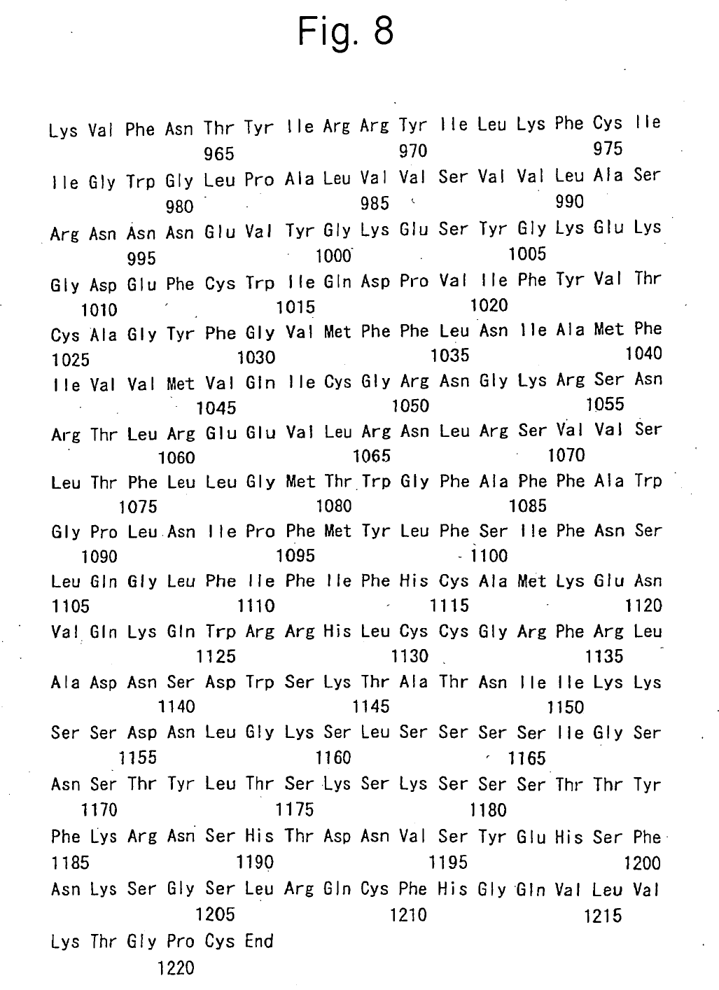

For the PCR reaction liquid, 1 μl cDNA solution, 0.5 μl F1 (10 μM), 0.5 μl R1 (10 μM), 2.5 μl enclosed 10× reaction liquid, 2.5 μl dNTP (10 mM), 0.5 μl ExTaq (Takara) and 17.5 μl Ootsuka distilled water were added for a total of 25 μl. The reaction liquid was PCR reacted using a ThermalCycler 9600. The PCR conditions were denaturing for 2 minutes at 95° C. followed by 35 cycles of 10 seconds at 98° C., 20 seconds at 63° C. and 180 seconds at 72° C. After amplification of about 3700 bp of PCR product was confirmed by electrophoresis using part of the PCR product, PCR product was purified using a QIAGEN PCR purification kit and subcloned to a TA vector (pCR2.1TOPO), and sequencing was performed to obtain the sequence (SEQ ID NO: 8) shown in FIGS. 3 and 4. The amino acid sequence anticipated from the DNA sequence of FIGS. 3 and 4 is shown in FIGS. 5 through 8 (SEQ ID NO: 9).

### Example 4

### cAMP Production Increase Activity Caused by Plant Lectins in dJ/GFP-Expressing CHO Cells

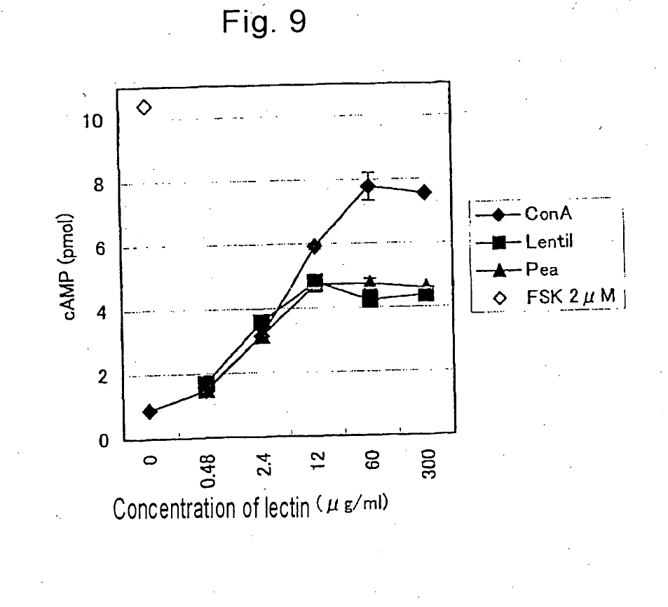

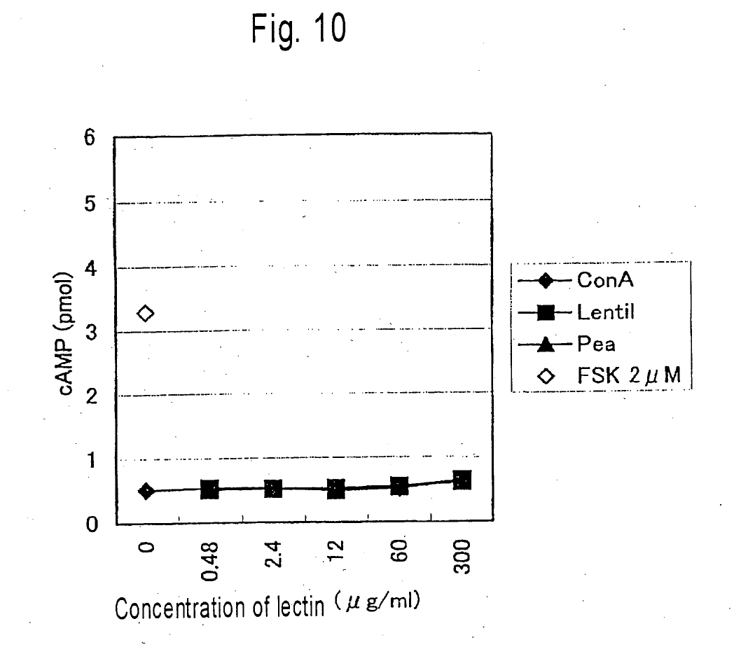

The CHO cell line used in the experiment was CHO-dJ287G14.2-GFP, which stably expresses a fused protein of GFP and the dJ287G14.2 obtained in Example 1. CHO-dJ287G14.2-GFP cells or mock CHO cells transfected with pAKKO-111H were seeded to a concentration of 2×10⁴/well on 96-well plates cultured overnight, and used to measure cAMP production. HBSS (Hank's balanced salt solution, Gibco) with 0.1% bovine serum albumin and 0.2 mM 3-Isobutyl-1-methylxanthine (IBMX, Sigma) added was used as the assay buffer. The cells were washed twice in assay buffer, and pre-incubated for 30 minutes at 37° C. The cells were washed twice, and samples of lectins and the like diluted with assay buffer were added and incubated for 30 minutes. The culture supernatant was discarded, and cAMP production was measured by a cAMP Screen System (ABI). As shown in FIG. 9, the results show concentration-dependent increases in cAMP production due to addition of ConA (Concanavalin A, Wako) Lentil Lectin (Wako) and Pea Lectin (Wako) in CHO-dJ287G14.2-GFP. As shown in FIG. 10, however, there was no increase in cAMP production due to addition of the aforementioned lectins in mock CHO cells.

### Example 5

### Movement Inside the Cell of dJ287G14.2-GFP Protein made to be Expressed in CHO Cells Due to Addition of Concanavalin A

An expression plasmid was constructed for expressing a fused protein consisting of Green Fluorescent Protein (GFP) cDNA isolated together with the translation frame from Aequoria victoria attached to the C terminus of dJ287G14.2. A fragment cut from GFP expression vector pQB 125 (Takara Shuzo) was used as the GFP cDNA. The termination codon of dJ287G14.2 was modified by PCR to a recognition sequence for reductase XbaI, the GFP fragment was ligated thereto, and the expression vector pAKKO-111H described in Example 1 was inserted. As a result, a plasmid of a dJ287G14.2 and GFP fused protein (hereunder, dJ287G14.2-GFP) expression vector was constructed. Next, this dJ287G14.2-GFP expression vector plasmid was introduced by gene transfer (Wako Pure Chemical) into CHOdhfr⁻ cells. Two days later selection medium was substituted, cells were selected from the proliferated transformants using GFP expression as the marker, and a CHO cell line CHO-dJ287G14.2-GFP expressing the dJ287G14.2-GFP fused protein was established.

Expression of the dJ287G14.2-GFP fused protein in the cells was located by sowing CHO-dJ287G14.2-GFP in a Lab-Tek II cover glass chamber (Nalgen Nunc), culturing them overnight under conditions of 37° C., 5% CO₂, substituting a medium for confocal microscopy (Hanks' Balanced Salt Solution (GIBCO BRL)), and observing GFP fluorescence with a confocal microscope (Leica). The medium for confocal microscopy was replaced with medium having sword bean-derived lectin and concanavalin A (ConA, Wako Pure Chemical) added at concentrations of 60 μg/ml, and the cells were observed after reacting for 2 hours at 37° C. GFP excitation was performed at 488 nM.

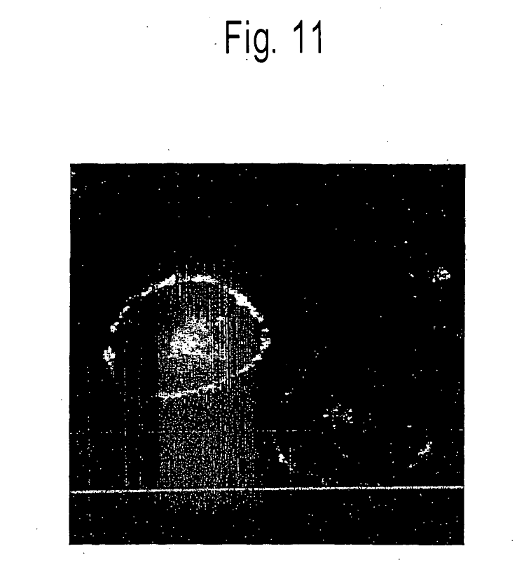

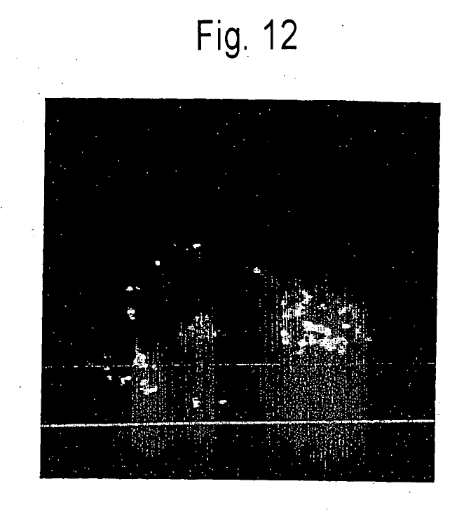

As a result, dJ287G14.2-GFP fused protein was seen to be expressed in cell membranes (FIG. 11). When these cells were cultured for two hours in medium with ConA added, GFP fluorescence was found to be moving not into the cell membranes but into the cytoplasm (FIG. 12). This indicated not only that dJ287G14.2 is a G protein-coupled receptor which is expressed in cell membranes, but also that dJ287G14.2 moves into the cytoplasm in response to ConA, or in other words that it is internalized by ConA.

### Example 6

### Cloning of mouse dJ287G14.2 Counterpart

An exon encoding mouse dJ287G14.2 was searched using the Celera mouse genome database, and based on this sequence translation region amplification primers, 5′ RACE primers and 3′ RACE primers were prepared. Amplification of the translation region was performed by PCR using primers (SEQ ID NOS: 29, 30, 31, 32), mouse spleen, lung, and 14-day fetal cDNA (Marathon-ready cDNA, Clontech) as the templates and PyroBEST DNA polymerase (Takara). Amplification of 3′ RACE was performed using gene-specific primers (SEQ ID NOS: 33, 34), mouse spleen cDNA (Marathon-ready cDNA) as the template and Advantage-2 polymerase mix (Clontech). Amplification of 5′ RACE was performed by the oligo-cap method using gene-specific primers (SEQ ID NOS: 35, 36, 37), mouse placental cDNA (Cap site cDNA dT Mouse Placenta, NIPPON GENE) as the template and Gene TaqNT (NIPPON GENE). From these results it is shown that mouse dJ287G14.2 exists in two types wherein the sequence of the N terminal differs depending on the connections from the donor site of the first exon to the acceptor site of the second exon, and that it exists with the sixth exon skipped and with the 26^{th} exon skipped and the amino acid sequence of the C terminal altered, and ORFs (SEQ ID NOS: 14, 16, 18, 20, 22, 24, 26, 28) were obtained which encode mouse dJ287G14.2 (SEQ ID NOS: 13, 15, 17, 19, 21, 23, 25, 27) consisting of a total of 1165-1258 amino acid residues.

### Example 7

### Expression of G Protein-Coupled Receptor EDG Family mRNA in Vascular Cells

Total RNA was prepared according to the Isogen (Nippon Gene) manual from fixed quantities of normal human coronary vascular endothelial cells, normal human coronary vascular smooth muscle cells, normal human aortal vascular endothelial cells and normal human aortal vascular smooth muscle cells (purchased from Asahi Technoglass). A reaction was performed from 1 μg of RNA at 42° C. using SuperScript II reverse transcriptase (GIBCO BRL) as the reverse transcriptase, according to the attached manual, followed after completion of the reaction by ethanol sedimentation and dissolution in TE (corresponding to 100 ng/μl RNA). Expression of mRNA of the EDG family was assayed using a Sequence Detection System Prism 7900HT system (Applied Biosystems). In order to assay the expressed amounts of the various receptors, TaqMan probes and primers, which specifically recognized the various receptors, were designed and synthesized using Primer Express (PE Applied Biosystems software). 5′-CCACCGACCCATGTACTATTTT-3′ (SEQ ID NO: 54), 5′-TGTAGGCTACTCCTGCCAACAG-3′ (SEQ ID NO: 55) and 5′-(Fam)-TTGGCAATCTGGCCCTCTCAGA-(Tamra)-3′ (SEQ ID NO: 56) as the TaqMan probe were used for detecting EDG-1, 5′-ACTGTCAGCACATGGCTCCTT-3′ (SEQ ID NO: 57), 5′-ACCGTAATGTGCCTCTCGATT-3′ (SEQ ID NO: 58) and 5′-(Fam)-ATTGACACCAGCCTGACGGCAT-(Tamra)-3′ (SEQ ID NO: 59) as the TaqMan probe for detecting EDG-2, 5′-CCGTGCTCTTCTTGGTCAT-3′ (SEQ ID NO: 60), 5′-CCAGATGGCAATCAAAACC-3′ (SEQ ID NO: 61) and 5′-(Fam)-TGCAGCTTCATCGTCTTGGAGAACCT-(Tamra)-3′ (SEQ ID NO: 62) as the TaqMan probe for detecting EDG-3, 5′-CCTGGTCAAGACTGTTGTCATC-3′ (SEQ ID NO: 63), 5′-CAGGACATTGCAGGACTCA-3′ (SEQ ID NO: 64) and 5′-(Fam)-TGGTACTGCTCCTGGATGGTTTAGGCT-(Tamra)-3′ (SEQ ID NO: 65) as the TaqMan probe for detecting EDG4, 5′-CCAACAAGGTCCAGGAACA-3′ (SEQ ID NO: 66), 5′-AGGTTTTCCACCACAATGG-3′ (SEQ ID NO: 67) and 5′-(Fam)-AATTATACCAAGGAGACGCTGGAAACGC-(Tamra)-3′ (SEQ ID NO: 68) as the TaqMan probe for detecting EDG-5, 5′-GAACTGCCTGTGCGCCTTT-3′ (SEQ ID NO: 69), 5′-CCATAGAGGCCCATGATGGT-3′ (SEQ ID NO: 70) and 5′-(Fam)-TCTGCCCCTCTACTCCAAGCGCTACATC-(Tamra)-3′ (SEQ ID NO: 71) as the TaqMan probe for detecting EDG-6, 5′-TGACTGCTTCCCTCACCAA-3′ (SEQ ID NO: 72), 5′-GCATCCTCATGATTGACATGTG-3′ (SEQ ID NO: 73) and 5′-(Fam)-TTGCTGGTTATCGCCGTGGAGA-(Tamra)-3′ (SEQ ID NO: 74) as the TaqMan probe for detecting EDG-7, or 5′-CTTGCTCCACTGTCTTGCC-3′ (SEQ ID NO: 75), 5′-TAGAGTGCACAGATCGCGG-3′ (SEQ ID NO: 76) and 5′-(Fam)-CTCTACGCCAAGGCCTACGTGCTCTTCT-(Tamra)-3′ (SEQ ID NO: 77) as the TaqMan probe for detecting EDG-8.

The reaction liquid for assay was prepared by adding the respective G protein-coupled receptor primers (0.9 μM), probe (0.25 μM) and cDNA corresponding to 25 ng total RNA according to the manual for the TaqMan Universal PCR Master Mix (Applied Biosystems). The PCR reaction was performed in 40 cycles of 2 minutes at 50° C. and 10 minutes at 95° C. followed by 15 seconds at 95° C. and 1 minute at 60° C. In normal human coronary vascular endothelial cells and normal human aortal vascular endothelial cells, EDG-1 exhibited the highest expression of all of the 354 G protein-coupled receptor assayed—1,676,305 copies/25 ng total RNA and 1,017,396 copies/25 ng total RNA, respectively. Likewise, in normal human coronary vascular smooth muscle cells and normal human aortal vascular smooth muscle cells, EDG-2 exhibited the highest expression of all the 354 G protein-coupled receptors assayed—148,922 copies/25 ng total RNA and 310,544 copies/25 ng total RNA, respectively. EDG-3, 4, 5, 6, 7 and 8 exhibited much less expression than EDG-1 and EDG-2. These results indicate that vascular endothelial cells are suitable for screening agonists and antagonists to EDG-1, and vascular smooth muscle cells for screening agonists and antagonists to EDG-2.

### Industrial Applicability

With the analysis method and assay kit of the present invention, a system is provided which can detect the presence or absence of target mRNA and the transcripted amount thereof in a sample containing multiple types of mRNA with high sensitivity in one operation. Consequently, with the present invention it is possible to provide a system, which can rapidly perform expression analysis of target genes with high sensitivity.

Moreover, by selecting as the target mRNA gene mRNA belonging to multiple G protein-coupled receptor, tyrosine kinase receptor, ion channel and other families, it is possible to rapidly and with high sensitivity specify disease genes associated with genes and the like belonging to the G protein-coupled receptor, tyrosine kinase receptor, ion channel and other families.

With the diagnostic method of the present invention, it is possible to diagnose with high precision a disease from which a patient suffers using an mRNA sample collected from that patient.

A receptor protein or partial peptide or salt thereof of the present invention or DNA encoding a receptor protein or partial peptide or salt thereof of the present invention is useful as a preventative and/or therapeutic drug for prostate cancer and other cancers (non-small cell carcinoma, ovarian cancer, stomach cancer, bladder cancer, breast cancer, cervical cancer, colon cancer, rectal cancer, large intestinal cancer and the like), prostatomegaly, male gonad dysfunction, infertility, premature birth, endometriosis, cirrhosis of the liver, hepatitis, hepatic insufficiency, calculus and the like.

Also, by using a receptor protein or partial peptide or salt thereof together with a protein (such as a lectin) having an affinity for a sugar chain which is one ligand, it is possible to efficiently screen compounds which alter the binding properties of the ligand with the dJ287G14.2 receptor or partial peptide or salt thereof of the present invention.

Moreover, EDG1 receptor agonists and antagonists can be efficiently screened using vascular endothelial cells, and EDG2 receptor agonists and antagonists using vascular smooth muscle cells.

1. A method for analyzing gene expression wherein genes whose expression is characteristically promoted or inhibited in certain cells or tissue, are identified by quantitatively analyzing the individual expressed amounts of multiple genes collectively.

2. The method according to claim 1, wherein expression analysis is performed collectively on a plurality of genes belonging to a specific gene family so that the genes in that family whose expression is characteristically promoted or inhibited in certain cells or tissues are identified by computing expressed amounts as absolute values.

3. The method according to claim 1, wherein an amplification reaction is performed by bringing mRNA sample which may contain a plurality of mRNA targets into contact at a plurality of reaction sites with individual amplification reagents each of which comprises a primer pair corresponding to a particular mRNA target, and gene expression analysis is performed by measuring the amounts produced in the resulting amplification product.

4. The method according to claim 1, wherein an amplification reaction is performed by bringing mRNA sample which may contain multiple mRNA targets into contact, at the reaction sites of a reaction device having multiple reaction sites, with individual amplification reagents each of which comprises a primer pair corresponding to a particular mRNA target, and gene expression analysis is performed by measuring the amounts produced in the resulting amplification product.

5. The method according to claim 2, wherein the specific gene family is the G protein-coupled receptor gene family.

6. The method according to claim 2, wherein the specific gene family is the tyrosine kinase receptor gene family.

7. The method according to claim 2, wherein the specific gene family is the ion channel gene family.

8. The method according to claim 2, wherein the specific gene family is a gene family associated with any of transcription factors, transporters, protein kinases, protein phosphatases, proteases, heat shock proteins, ATPases and DNA-binding proteins.

9. A drug comprising a gene or product of a gene, which is specified by a method described in any of claims 1 through 8, and the expression of which is characteristically promoted or inhibited in certain cells or tissue.

10. The method according to claim 3 or 4, wherein the reaction device is a plate having a plurality of wells as reaction sites.

11. The method according to claim 10, wherein the plate is a 96-well or 384-well plate.

12. The method according to claim 3 or 4, wherein 10 to 800 primer pairs are used.

13. The method according to claim 3 or 4, wherein 10 to 300 primer pairs are used.

14. The method according to claim 3, 4, 12 or 13, wherein the amplification reaction is a polymerase chain reaction.

15. The method according to claim 14, wherein SNP analysis is performed.

16. The method according to claim 3 or 4, wherein the produced amounts of amplification products are measured using probes which are complementary or substantially complementary to said amplification products.

17. The method according to claim 16, wherein the probes are probes which hybridize with mRNA.

18. The method according to claim 17, wherein the probes are fluorescence labeled probes.

19. The method according to claim 3 or 4, wherein normal human-derived mRNA sample and mRNA sample derived from a patient with a specific disease are used as the mRNA samples.

20. The method according to claim 19, wherein mRNA whose expression is promoted or inhibited in mRNA sample derived from a disease patient is specified, and a gene encoding said mRNA is designated as a disease-associated gene for that disease.

21. The method according to claim 20, wherein the specific gene family is the G protein-coupled receptor protein gene family, and wherein a cancer-related gene is specified by using mRNA samples derived from a cancer patient.

22. A primer pair kit comprising two or more pairs of primers each consisting of a first primer which is complementary or substantially complementary to one chain of an exon region of a target gene sequence and a second primer which is complementary or substantially complementary to the other chain of the exon region of the target gene sequence.

23. The primer pair kit according to claim 22, wherein the target gene is a human G protein-coupled receptor protein gene, tyrosine kinase receptor gene or ion channel gene.

24. The primer pair kit according to claim 23, composed of 10 to 800 primer pairs.

25. The primer pair kit according to claim 23, composed of 10 to 300 primer pairs.

26. A usage of the primer pair kit according to claim 23, for purposes of specifying disease-associated genes.

27. An mRNA assay kit having each reaction site in a reaction device with a plurality of reaction sites filled with a respective amplification reagent comprising a primer pair corresponding to a particular mRNA target.

28. The kit according to claim 27, also comprising a fluorescent probe.

29. The kit according to claim 28, also comprising Tth DNA polymerase,

30. A method of diagnosing a patient's disease using the method according to any of claims 1 through 4 or the assay kit according to claim 27 by assaying the mRNA of a plurality of target disease genes which may be contained in an mRNA sample collected from the patient, or by measuring the mutated amount of said mRNA.

31. The diagnostic method according to claim 30, wherein cancer is diagnosed by identifying cancer-associated human G protein-coupled receptor protein genes.

32. A drug comprising an agonist, antagonist or antibodies to the gene product of a gene identified by the diagnostic method of claim 30, or DNA encoding said gene product.

33. The drug according to claim 32, which is a cancer therapy drug.

34. A method of treating diseases involving a gene identified by the diagnostic method according to claim 30 by administering an agonist, antagonist or antibodies to the gene product of said gene or DNA encoding said gene product.

35. The treatment method according to claim 34, wherein the disease is cancer.

36. A drug containing a receptor protein which contains an amino acid sequence identical to or substantially identical to the amino acid sequence represented by SEQ ID NO:9, or a partial peptide or salt thereof.

37. The drug according to claim 36 which is a birth inducer or a preventative and/or therapeutic drug for cancer, prostatomegaly, male gonad dysfunction, infertility, premature birth, endometriosis, cirrhosis of the liver, hepatitis, hepatic insufficiency or calculus.

38. The drug according to claim 37 wherein the cancer is prostate cancer, non-small cell carcinoma, ovarian cancer, stomach cancer, bladder cancer, breast cancer, cervical cancer, colon cancer, rectal cancer or large intestinal cancer.

39. A diagnostic drug containing antibodies to a protein which contains an amino acid sequence identical to or substantially identical to the amino acid sequence represented by SEQ ID NO:9, or to a partial peptide or salt thereof.

40. The diagnostic drug according to claim 39 which is a diagnostic drug for cancer, prostatomegaly, male gonad dysfunction, infertility, premature birth, endometriosis, cirrhosis of the liver, hepatitis, hepatic insufficiency or calculus.

41. A drug containing antibodies to a protein which contains an amino acid sequence identical to or substantially identical to the amino acid sequence represented by SEQ ID NO:9, or to a partial peptide or salt thereof.

42. The drug according to claim 41 which is a birth inducer or a preventative and/or therapeutic drug for cancer, prostatomegaly, male gonad dysfunction, infertility, premature birth, endometriosis, cirrhosis of the liver, hepatitis, hepatic insufficiency or calculus.

43. A method of screening compounds or salts of compounds which alter the binding properties of a G protein-coupled receptor protein containing an amino acid sequence identical to or substantially identical to the amino acid sequence represented by SEQ ID NO:9 or of a salt thereof with a protein showing affinity for a sugar chain, characterized by the use of (1) said G protein-coupled receptor protein, or a partial peptide or salt thereof, and (2) the protein showing affinity for a sugar chain.

44. The screening method according to claim 43, wherein the protein showing affinity for a sugar chain is a protein showing affinity for an asparagine-linked sugar chain or a serine/threonine-linked sugar chain.

45. The screening method according to claim 43, wherein the protein showing affinity for a sugar chain is a lectin.

46. The screening method according to claim 43, wherein the protein showing affinity for a sugar chain is concanavalin A, lentil lectin, pea lectin, datura stramonium lectin, sophora japonica lectin or phytohemagglutinin.

47. A screening kit for compounds or salts of compounds which alter the binding properties of a G protein-coupled receptor protein containing an amino acid sequence identical to or substantially identical to the amino acid sequence represented by SEQ ID NO:9 or of a salt thereof with a protein showing affinity for a sugar chain, characterized in containing (1) the aforementioned G protein-coupled receptor protein, or a partial peptide or salt thereof, and (2) the protein showing affinity for a sugar chain.

48. A compound or salt of a compound which alters the binding properties of a protein showing affinity for a sugar chain with a G protein-coupled receptor protein containing an amino acid sequence identical to or substantially identical to the amino acid sequence represented by SEQ ID NO: 9 or with a salt thereof, obtained using the screening method according to claim 46 or the screening kit according to claim 47.

49. A drug containing a compound or salt thereof according to claim 48.

50. The drug according to claim 49 which is a birth inducer or a preventative and/or therapeutic drug for cancer, prostatomegaly, male gonad dysfunction, infertility, premature birth, endometriosis, cirrhosis of the liver, hepatitis, hepatic insufficiency or calculus.

51. The drug according to claim 50 wherein the cancer is prostate cancer, non-small cell carcinoma, ovarian cancer, stomach cancer, bladder cancer, breast cancer, cervical cancer, colon cancer, rectal cancer or large intestinal cancer.

52. A method of screening birth inducers or compounds or salts of compounds for preventing and/or treating cancer, prostatomegaly, male gonad dysfunction, infertility, premature birth, endometriosis, cirrhosis of the liver, hepatitis, hepatic insufficiency or calculus by altering the expressed amount of a G protein-coupled receptor protein containing an amino acid sequence identical to or substantially identical to the amino acid sequence represented by SEQ ID NO: 9, characterized by the use of a polynucleotide containing a polynucleotide encoding said G protein-coupled receptor protein or a partial peptide thereof.

53. A screening kit for screening birth inducers or compounds or salts of compounds for preventing and/or treating cancer, prostatomegaly, male gonad dysfunction, infertility, premature birth, endometriosis, cirrhosis of the liver, hepatitis, hepatic insufficiency or calculus by altering the expressed amount of a G protein-coupled receptor protein containing an amino acid sequence identical to or substantially identical to the amino acid sequence represented by SEQ ID NO: 9, said screening kit containing a polynucleotide containing a polynucleotide encoding said G protein-coupled receptor protein or a partial peptide thereof.

54. A birth inducer or compound or salt of a compound for preventing and/or treating cancer, prostatomegaly, male gonad dysfunction, infertility, premature birth, endometriosis, cirrhosis of the liver, hepatitis, hepatic insufficiency or calculus by altering the expressed amount of a G protein-coupled receptor protein containing an amino acid sequence identical to or substantially identical to the amino acid sequence represented by SEQ ID NO: 9 or of a partial peptide thereof, that can be obtained using the screening method according to claim 52 or the screening kit according to claim 53,

55. A birth inducer or preventative and/or therapeutic agent for cancer, prostatomegaly, male gonad dysfunction, infertility, premature birth, endometriosis, cirrhosis of the liver, hepatitis, hepatic insufficiency or calculus, containing a compound or salt thereof according to claim 54.

56. The agent according to claim 55 wherein the cancer is prostate cancer, non-small cell carcinoma, ovarian cancer, stomach cancer, bladder cancer, breast cancer, cervical cancer, colon cancer, rectal cancer or large intestinal cancer.

57. A signal transmission enhancing agent for a G protein-coupled receptor protein containing an amino acid sequence identical to or substantially identical to the amino acid sequence represented SEQ ID NO: 9, containing a protein showing affinity for a sugar chain.

58. The agent according to claim 57, which is a birth inducer or preventative and/or therapeutic agent for cancer, prostatomegaly, male gonad dysfunction, infertility, premature birth, endometriosis, cirrhosis of the liver, hepatitis, hepatic insufficiency or calculus.

59. The agent according to claim 58, wherein the cancer is prostate cancer, non-small cell carcinoma, ovarian cancer, stomach cancer, bladder cancer, breast cancer, cervical cancer, colon cancer, rectal cancer or large intestinal cancer.

60. A birth inducer or preventative and/or therapeutic agent for cancer, prostatomegaly, male gonad dysfunction, infertility, premature birth, endometriosis, cirrhosis of the liver, hepatitis, hepatic insufficiency or calculus, containing a polynucleotide which contains a polynucleotide encoding a G protein-coupled receptor protein containing an amino acid sequence identical to or substantially identical to the amino acid sequence represented by SEQ ID NO: 9, or a partial peptide thereof.

61. The agent according to claim 60, wherein the cancer is prostate cancer, non-small cell carcinoma, ovarian cancer, stomach cancer, bladder cancer, breast cancer, cervical cancer, colon cancer, rectal cancer or large intestinal cancer.

62. A diagnostic drug for cancer, prostatomegaly, male gonad dysfunction, infertility, premature birth, endometriosis, cirrhosis of the liver, hepatitis, hepatic insufficiency or calculus, containing a polynucleotide which contains a polynucleotide encoding a G protein-coupled receptor protein containing an amino acid sequence identical to or substantially identical to the amino acid sequence represented by SEQ ID NO: 9, or a partial peptide thereof.

63. The diagnostic drug according to claim 62, wherein the cancer is prostate cancer, non-small cell carcinoma, ovarian cancer, stomach cancer, bladder cancer, breast cancer, cervical cancer, colon cancer, rectal cancer or large intestinal cancer.

64. A birth inducer or preventative and/or therapeutic agent for cancer, prostatomegaly, male gonad dysfunction, infertility, premature birth, endometriosis, cirrhosis of the liver, hepatitis, hepatic insufficiency or calculus, containing an antisense polynucleotide which contains a nucleotide sequence or part of a nucleotide sequence complementary to a polynucleotide containing a polynucleotide which encodes a G protein-coupled receptor protein containing an amino acid sequence identical to or substantially identical to the amino acid sequence represented by SEQ ID NO: 9, or a partial peptide thereof.

65. The agent according to claim 64, wherein the cancer is prostate cancer, non-small cell carcinoma, ovarian cancer, stomach cancer, bladder cancer, breast cancer, cervical cancer, colon cancer, rectal cancer or large intestinal cancer.

66. A diagnostic drug for cancer, prostatomegaly, male gonad dysfunction, infertility, premature birth, endometriosis, cirrhosis of the liver, hepatitis, hepatic insufficiency or calculus, containing an antisense polynucleotide which contains a nucleotide sequence or part of a nucleotide sequence complementary to a polynucleotide containing a polynucleotide which encodes a G protein-coupled receptor protein containing an amino acid sequence identical to or substantially identical to the amino acid sequence of SEQ ID NO: 9, or a partial peptide thereof.

67. The diagnostic drug according to claim 66, which is a diagnostic drug for cancer.

68. The diagnostic drug according to claim 67, wherein the cancer is prostate cancer, non-small cell carcinoma, ovarian cancer, stomach cancer, bladder cancer, breast cancer, cervical cancer, colon cancer, rectal cancer or large intestinal cancer.

69. A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized by administering, to mammals, an effective dose of a receptor protein containing an amino acid sequence identical to or substantially identical to the amino acid sequence represented by SEQ ID NO: 9, or a partial peptide or salt thereof.

70. A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized by administering, to mammals, an effective dose of antibodies to a protein containing an amino acid sequence identical to or substantially identical to the amino acid sequence represented by SEQ ID NO:9, or to a partial peptide or salt thereof.

71. A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized by administering, to mammals, an effective dose of a compound or salt thereof which alters the binding properties of a G protein-coupled receptor protein containing an amino acid sequence identical to or substantially identical to the amino acid sequence represented by SEQ ID NO: 9, or of a partial peptide or salt thereof, with a protein showing affinity for a sugar chains.

72. A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized by administering, to mammals, an effective dose of a compound or salt thereof which alters the expressed amount of a G protein-coupled receptor protein containing an amino acid sequence identical to or substantially identical to the amino acid sequence represented by SEQ ID NO: 9.

73. A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized by administering, to mammals, an effective dose of a protein or salt thereof showing affinity for a sugar chain.

74. A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized by administering, to mammals, an effective dose of an antisense polynucleotide containing a nucleotide sequence or part of a nucleotide sequence complementary to a polynucleotide encoding a protein or partial peptide of a protein containing an amino acid sequence identical to or substantially identical to the amino acid sequence represented SEQ ID NO: 9.

75. A method of preventing and/or treating cancer, prostatomegaly, male gonad dysfunction, infertility, premature birth, endometriosis, cirrhosis of the liver, hepatitis, hepatic insufficiency or calculus, or a method of inducing birth, characterized by administering, to mammals, an effective dose of a polynucleotide containing a polynucleotide which encodes a G protein-coupled receptor protein containing an amino acid sequence identical to or substantially identical to the amino acid sequence represented by SEQ ID NO: 9, or a partial peptide thereof.

76. A usage of a receptor protein containing an amino acid sequence identical to or substantially identical to the amino acid sequence represented by SEQ ID NO: 9, or a partial peptide or salt thereof, for manufacturing a birth inducer or a preventative and/or therapeutic agent for cancer, prostatomegaly, male gonad dysfunction, infertility, premature birth, endometriosis, cirrhosis of the liver, hepatitis, hepatic insufficiency or calculus.

77. A usage of antibodies to a receptor protein containing an amino acid sequence identical to or substantially identical to the amino acid sequence represented by SEQ ID NO: 9, or to a partial peptide or salt thereof, for manufacturing a birth inducer or a preventative and/or therapeutic agent for cancer, prostatomegaly, male gonad dysfunction, infertility, premature birth, endometriosis, cirrhosis of the liver, hepatitis, hepatic insufficiency or calculus.

78. A usage of a compound or salt thereof which alters the binding properties of a protein showing affinity for a sugar chain with a G protein-coupled receptor protein containing an amino acid sequence identical to or substantially identical to the amino acid sequence represented by SEQ ID NO: 9, or with a partial peptide or salt thereof, for manufacturing a birth inducer or a preventative and/or therapeutic agent for cancer, prostatomegaly, male gonad dysfunction, infertility, premature birth, endometriosis, cirrhosis of the liver, hepatitis, hepatic insufficiency or calculus.

79. A usage of a compound or salt thereof which alters the expressed amount of a G protein-coupled receptor protein containing an amino acid sequence identical to or substantially identical to the amino acid sequence represented by SEQ ID NO: 9, for manufacturing a birth inducer or a preventative and/or therapeutic agent for cancer, prostatomegaly, male gonad dysfunction, infertility, premature birth, endometriosis, cirrhosis of the liver, hepatitis, hepatic insufficiency or calculus.

80. A usage of a protein or salt thereof showing affinity for a sugar chain for manufacturing a birth inducer or a preventative and/or therapeutic agent for cancer, prostatomegaly, male gonad dysfunction, infertility, premature birth, endometriosis, cirrhosis of the liver, hepatitis, hepatic insufficiency or calculus.

81. A usage of an antisense polynucleotide containing a nucleotide sequence or part of a nucleotide sequence complementary to a polynucleotide which encodes a protein containing an amino acid sequence identical to or substantially identical to the amino acid sequence represented by SEQ ID NO: 9 or a partial peptide thereof, for manufacturing a birth inducer or a preventative and/or therapeutic agent for cancer, prostatomegaly, male gonad dysfunction, infertility, premature birth, endometriosis, cirrhosis of the liver, hepatitis, hepatic insufficiency or calculus.

82. A usage of a polynucleotide containing a polynucleotide encoding a G protein-coupled receptor protein containing an amino acid sequence identical to or substantially identical to the amino acid sequence represented by SEQ ID NO: 9 or a partial peptide thereof, for manufacturing a birth inducer or a preventative and/or therapeutic agent for cancer, prostatomegaly, male gonad dysfunction, infertility, premature birth, endometriosis, cirrhosis of the liver, hepatitis, hepatic insufficiency or calculus.

83. A G protein-coupled receptor protein containing an amino acid sequence identical to or substantially identical to the amino acid sequence represented by SEQ ID NO: 13, SEQ ID NO: 15, SEQ ID NO: 17, SEQ ID NO: 19, SEQ ID NO: 21, SEQ ID NO: 23, SEQ ID NO: 25 or SEQ ID NO: 27, or a salt thereof.

84. A G protein-coupled receptor protein consisting of an amino acid sequence represented by SEQ ID NO: 13, SEQ ID NO: 15, SEQ ID NO: 17, SEQ ID NO: 9, SEQ ID NO: 21, SEQ ID NO: 23, SEQ ID NO: 25 or SEQ ID NO: 27, or a salt thereof.

85. A polynucleotide containing the polynucleotide encoding the G protein-coupled receptor protein according to claim 81.

86. DNA consisting of a nucleotide sequence represented by SEQ ID NO: 14, SEQ ID NO: 16, SEQ ID NO: 18,.SEQ ID NO: 20, SEQ ID NO: 22, SEQ ID NO: 24, SEQ ID NO: 26 or SEQ ID NO: 28.

87. A recombinant vector containing the polynucleotide according to claim 83.

88. A transformant transformed by the recombinant vector according to claim 87.

89. A method of manufacturing the G protein-coupled receptor protein or salt thereof according to claim 83, wherein the transformant according to claim 88 is cultured in order to produce the G protein-coupled receptor protein or salt thereof according to claim 83.

90. Antibodies to the G protein-coupled receptor protein or salt thereof according to claim 83.

91. DNA which hybridizes under highly stringent conditions with DNA consisting of a nucleotide sequence represented by SEQ ID NO: 14, SEQ ID NO: 16, SEQ ID NO: 18, SEQ ID NO: 20, SEQ ID NO: 22, SEQ ID NO: 24, SEQ ID NO: 26 or SEQ ID NO: 28.

92. A polynucleotide containing a nucleotide sequence or part of a nucleotide sequence complementary to the polynucleotide according to claim 85.

93. A method for screening EDG-1 receptor agonists or antagonists, characterized by the use of vascular endothelial cells.

94. The screening method according to claim 93, wherein the EDG-1 receptor is a G protein-coupled receptor protein containing an amino acid sequence identical to or substantially identical to the amino acid sequence represented by SEQ ID NO: 38, or a partial peptide or salt thereof.

95. A screening kit for EDG-1 receptor agonists or antagonists, characterized by containing vascular endothelial cells.

96. An EDG-1 receptor agonist or antagonist obtained using the screening method according to claim 93 or the screening kit according to claim 95.

97. A preventative and/or therapeutic agent for arteriosclerosis, myocardial infarction, cerebral infarction or ischemic disease, containing an EDG-1 receptor agonist obtained using the screening method according to claim 93 or the screening kit according to claim 85.

98. A method of screening EDG-2 receptor agonists or antagonists, characterized by the use of vascular smooth muscle cells.

99. The screening method according to claim 98 wherein the EDG-2 receptor is a G protein-coupled receptor protein containing an amino acid sequence identical to or substantially identical to the amino acid sequence represented by SEQ ID NO: 40, or a partial peptide or -salt thereof.

100. A kit for screening EDG-2 receptor agonists or antagonists, characterized by containing vascular smooth muscle cells.

101. An EDG-2 receptor agonist or antagonist obtained using the screening method according to claim 98 or the screening kit according to claim 100.

102. A preventative and/or therapeutic agent for arteriosclerosis, myocardial infarction, cerebral infarction or ischemic disease, containing an EDG-2 receptor agonist or antagonist obtained using the screening method according to claim 98 or the screening kit according to claim 100.

103. A method of preventing and/or treating arteriosclerosis, myocardial infarction, cerebral infarction or ischemic disease, characterized by administering, to mammals, an effective dose f an EDG-1 receptor agonist obtained using the screening method according to claim 93 or the screening kit according to claim 95, or an EDG-2 receptor antagonist obtained using the screening method according to claim 98 or the screening kit according to claim 100.

104. A usage of an EDG-1 receptor agonist obtained using the screening method according to claim 93 or the screening kit according to claim 95 or an EDG-2 receptor antagonist obtained using the screening method according to claim 98 or the screening kit according to claim 100, for manufacturing a preventative and/or therapeutic agent for arteriosclerosis, myocardial infarction, cerebral infarction or ischemic disease.
